(19) United States
(12) Patent Application Publication
(10) Pub. No.: US 2013/0190357 A1
(43) Pub. Date: Jul. 25, 2013

(54) COMPOSITIONS AND METHODS FOR ASSESSING AND TREATING A PRECURSOR LESION AND/OR ESOPHAGEAL CANCER

(71) Applicant: Charis Eng, Cleveland Heights, OH (US)

(72) Inventor: Charis Eng, Cleveland Heights, OH (US)

(21) Appl. No.: 13/559,101

(22) Filed: Jul. 26, 2012

Related U.S. Application Data
(60) Provisional application No. 61/511,782, filed on Jul. 26, 2011.

(51) Int. Cl. C12Q 1/68; C07K 16/18; G01N 33/68; C07K 14/705; C07K 14/47; C07K 16/30

(52) U.S. Cl. CPC C12Q 1/6886; C07K 14/4748; C07K 16/30; G01N 33/6893; C07K 14/70596; C07K 16/18
USPC 514/338; 530/350; 536/23.5; 530/387.9; 435/6.11; 436/501

(57) ABSTRACT

One aspect of the present disclosure relates to a method for predicting a subject's risk of developing an esophageal cancer, a precursor lesion, or both. One step of the method includes obtaining a biological sample from the subject. Next, the presence of at least one germline mutation is determined in the biological sample. The subject is at an increased risk of an esophageal cancer, a precursor lesion, or both, where the presence of at least one germline mutation is determined.

### RELATED APPLICATION

This application claims the benefit of U.S. Provisional Patent Application Ser. No. 61/511,782, filed Jul. 26, 2011, the entirety of which is hereby incorporated by reference for all purposes.

### TECHNICAL FIELD

The present disclosure relates generally to compositions and methods for assessing and treating a precursor lesion and/or an esophageal cancer, and more particularly to compositions and methods for predicting, preventing, and/or treating Barrett's esophagus and/or esophageal adenocarcinoma.

### BACKGROUND

The incidence of esophageal adenocarcinoma (EAC) in the United States and Europe has increased 350% since 1970, with uncertain etiology. Although early-stage EAC is curable, most cases are detected at an advanced stage with poor survival. Esophageal adenocarcinoma is believed to be preceded by Barrett's esophagus (BE), a premalignant metaplasia caused by chronic gastroesophageal reflux disease (GERD). GERD-related inflammation and the transforming growth factor β (TGFB) pathway have been implicated in sporadic BE and EAC, just as the role of inflammation has become prominent in a range of human cancers; however, the role of inflammation in BE and EAC has not been thoroughly studied. Although most BE and EAC are believed to be sporadic, genetic (heritable) etiologies have been supported by observation of familial clustering of cases noted over several decades. An autosomal dominant mode of inheritance with incomplete penetrance is consistent with most published studies, and rare reported cases are consistent with autosomal recessive inheritance; yet, shared environmental factors may contribute to such familial aggregation.

### SUMMARY

One aspect of the present disclosure relates to an isolated nucleic acid molecule comprising a polynucleotide sequence selected from the group consisting of SEQ ID NO:7, SEQ ID SEQ ID NO:13, SEQ ID NO:17, SEQ ID NO:27, SEQ ID NO:28, SEQ ID NO:29, SEQ ID NO:30 and SEQ ID NO:37.

Another aspect of the present disclosure relates to an isolated polypeptide molecule comprising an amino acid sequence selected from the group consisting of SEQ ID NO:9, SEQ ID NO:10, SEQ ID NO:14, SEQ ID NO:18, SEQ ID NO:31, SEQ ID NO:32, SEQ ID NO:33, SEQ ID NO:34 and SEQ ID NO:38.

Another aspect of the present disclosure relates to an isolated antibody that specifically binds to a polypeptide molecule having an amino acid sequence selected from the group consisting of SEQ ID NO:9, SEQ ID NO:10, SEQ ID NO:14, SEQ ID NO:18, SEQ ID NO:31, SEQ ID NO:32, SEQ ID NO:33, SEQ ID NO:34 and SEQ ID NO:38.

Another aspect of the present disclosure relates to a method for predicting a subject's risk of developing an esophageal cancer, a precursor lesion, or both. One step of the method includes obtaining a biological sample from the subject. Next, the presence of at least one germline mutation is determined in the biological sample. The subject is at an increased risk of an esophageal cancer, a precursor lesion, or both, where the presence of at least one germline mutation is determined.

Another aspect of the present disclosure relates to a method for determining a treatment strategy for a subject. One step of the method includes predicting the subject's risk for developing an esophageal cancer, a precursor lesion, or both, by obtaining a biological sample from the subject and then determining the presence of at least one germline mutation in the biological sample. If the subject exhibits a low risk of developing an esophageal cancer, a precursor lesion, or both, a decision is made to perform a first therapeutic intervention. If the subject exhibits a high risk of developing an esophageal cancer, a precursor lesion, or both, a decision is made to perform a second therapeutic intervention.

Another aspect of the present disclosure relates to a method for treating a subject with an esophageal cancer, a precursor lesion, or both. One step of the method includes obtaining a biological sample from the subject. Next, the presence of at least one germline mutation in the biological sample is determined. A therapeutic intervention is then administered to the subject when the presence of one or more germline mutations is detected in the biological sample.

Another aspect of the present disclosure relates to a kit for predicting a subject's risk of developing an esophageal cancer, a precursor lesion, or both.

### BRIEF DESCRIPTION OF THE DRAWINGS

The foregoing and other features of the present disclosure will become apparent to those skilled in the art to which the present disclosure relates upon reading the following description with reference to the accompanying drawings, in which:

FIG. 1 is a schematic illustration showing a multistage strategy used to identify Barrett's esophagus/esophageal adenocarcinoma (BE/EAC) susceptibility genes via a genome-wide combined linkage-association analysis, followed by an independent genome-wide single-nucleotide polymorphism (SNP)-based case control validation;

FIGS. 2A-J shows fine mapping SNPs using genome-wide moving window haplotype analysis and association of haplotypes with BE/EAC (p<0.005) based on the validation case-control series. A haplotype window consists of a number of SNPs that are in tight linkage (pre-defined from 3-5 SNPs. We show 10 panels with haplotypes located in specific chromosomal regions that are associated with BE/EAC. At the top of each panel is an ideogram showing a chromosome and the location of the haplotype. Immediately below the ideogram is a scale that measures the location of the haplotype/SNP in base pairs. Below the scale are the respective haplotype windows. The pink horizontal bars are the haplotype windows containing 3 SNPs, the orange bars contain 4 SNPs and the red bar contains 5 SNPs. The downward green arrow shows the significant SNPs (p<0001), from single-SNP association analysis that intersects (is shared among) the overlapping significant haplotype windows (p<0005). Beneath the all the haplotypes are genes in alignment with the haplotypes. FIG. 2A. Haplotypes located on 1q21.2 and harboring significant SNP rs2809811; FIG. 2B. Haplotypes located on 1q24.2 to 1q25.3 and harboring significant SNPs rs6659944, rs3853181 in C1orf129 and rs6661125 in LHX4; FIG. 2C. Haplotypes located on 1q41 and harboring the significant SNP rs12070516 in MARK1; FIG. 2D. Haplotypes that are located on 8p22 and harbor the significant SNP rs381111, in the MSR1 gene; FIG. 2E. Haplotypes that are located on 8q22.1 and harbor and the significant SNP rs3097418 in TMEM67; FIG. 2F. Haplotypes that are located on 8q22.1-23.1 and harboring the significant SNPs rs3098233 in CTHRC1 and rs3098224 in WDSOF1; FIG. 2G. Haplotypes that are located on 8q24.2-24.22 and harbor the significant SNP rs4388439 in KCNQ3; FIG. 2H. Haplotypes that are located on 10q21.1 and harbor the significant SNP rs11001056 in PRKG1; FIG. 2I. Haplotypes located on 10q22.1 and harboring the significant SNP rs11000190 in ASCC1; FIG. 2J. Haplotypes located on 11q14 and harboring the significant SNP rs3924745;

FIG. 3 shows re-clustering of gene expression based on significant genes within the nine regions of interest and on the expression array dataset GDS3472;

FIGS. 4A-B are a series of chromatograms showing the germline MSR1 wild-type sequence (FIG. 4A) and the MSR1 exon 6 c.877C>T (p.R293X) mutation (FIG. 4B) that was observed in approximately 5% of BE and EAC cases, but not in any of the 139 controls (wild-type sequence, control) (heterozygous single-nucleotide variant is indicated by the arrow);

FIGS. 5A-C are a series of chromatograms showing mutations detected in MSR1 exon 5 (FIG. 5A), CTHRC1 exon 1 (FIG. 5B), and ASCC1 exon 8 (FIG. 5C) (in each of the three panels, the wild-type sequence is shown on the top and the mutant sequence is shown on the bottom);

FIGS. 6A-B are representative Western blots showing detection of MSR1 and CCND1 protein levels from lymphoblastoid cells derived from patients with BE and from population controls (FIG. 6A), and MSR1 and CCND1 protein levels after HEK293 cells were transiently transfected with empty vector or wild-type MSR1 constructs (FIG. 6B) (tubulin was used a loading control for FIGS. 6A-B); and

FIGS. 7A-B are a series of images showing hematoxylin-eosin staining of an esophageal lesion from a biopsy specimen displaying characteristic goblet cells from a representative patient with BE (FIG. 7A) and CCND1-positive staining (brown by immunoperoxidase) in the nuclei of BE lesion cells from a patient who was germline MSR1-mutation positive (FIG. 7B) (hematoxylin counterstain).

### DETAILED DESCRIPTION

Methods involving conventional molecular biology techniques are described herein. Such techniques are generally known in the art and are described in detail in methodology treatises, such as Current Protocols in Molecular Biology, ed. Ausubel et al., Greene Publishing and Wiley-Interscience, New York, 1992 (with periodic updates). Unless otherwise defined, all technical terms used herein have the same meaning as commonly understood by one of ordinary skill in the art to which the present disclosure pertains. Commonly understood definitions of molecular biology terms can be found in, for example, Rieger et al., Glossary of Genetics: Classical and Molecular, 5th Ed., Springer-Verlag: New York, 1991, and Lewin, Genes V, Oxford University Press: New York, 1994. The definitions provided herein are to facilitate understanding of certain terms used frequently herein and are not meant to limit the scope of the present disclosure.

In the context of the present disclosure, the term “esophageal carcinoma” can refer to intramucosal carcinoma and esophageal adenocarcinoma (EAC), or esophageal cancer.

As used herein, the term “precursor lesion” can refer to a premalignant cell, cell mass or condition (e.g., as determined by histological analysis), such as Barrett's esophagus (BE). BE can include both short segment and long segment forms.

As used herein, the terms “conditions of high stringency” or “high stringent hybridization conditions” can refer to any conditions in which hybridization will occur when there is at least about 85%, e.g., 90%, 95%, or 97% to 100%, complementarity (identity) between a target molecule (e.g., a nucleic acid or polypeptide of interest) and a probe.

As used herein, the terms “biological sample” or “specimen” can refer to a cell or fluid sample from a subject. In some instances, a biological sample can be derived from a subject that has been diagnosed with chronic gastroesophageal reflux disease, scleroderma, EAC, prior esophageal resection, BE, or an esophageal mucosa abnormality.

As used herein, the term “nucleic acid molecule” can refer to DNA molecules (e.g., cDNA or genomic DNA), RNA molecules (e.g., mRNA), and analogs of the DNA or RNA generated using nucleotide analogs. In some instances, a nucleic acid molecule can be single-stranded or double-stranded. In other instances, the nucleic acid molecule can comprise germline DNA. Thus, in one example, a germline DNA mutation can include a mutation that occurs in a germ cell (e.g., a sperm or egg cell) and is passed from one generation to the next. In further instances, the nucleic acid molecule can comprise somatic DNA. Thus, in another example, a somatic DNA mutation can include a mutation that occurs in a non-germ cell and is not passed on to future generations.

As used herein, the term “isolated” when referring to a nucleic acid molecule can refer to a nucleic acid molecule that is separated from other nucleic acid molecules present in the natural source of the nucleic acid. For example, with regard to genomic DNA, the term “isolated” can include nucleic acid molecules that are separated from the chromosome with which the genomic DNA is naturally associated. In some instances, an “isolated” nucleic acid can be free of sequences that naturally flank the nucleic acid (i.e., sequences located at the 5′ and 3′ ends of the nucleic acid) in the genomic DNA of the organism from which the nucleic acid is derived. In other instances, an “isolated” nucleic acid molecule can be substantially free of other cellular material or culture medium when produced by recombinant techniques, or substantially free of chemical precursors or other chemicals when chemically synthesized.

As used herein, the term “polypeptide” can refer to an oligopeptide, peptide, polypeptide, or protein sequence, or to a fragment, portion, or subunit of any of these, and to naturally occurring or synthetic molecules. The term “polypeptide” can also include amino acids joined to each other by peptide bonds or modified peptide bonds, i.e., peptide isosteres, and may contain any type of modified amino acids. The term can also include peptides and polypeptide fragments, motifs and the like, glycosylated polypeptides, and all “mimetic” and “peptidomimetic” polypeptide forms.

The present disclosure relates generally to compositions and methods for assessing and treating a precursor lesion, an esophageal cancer, or both, and more particularly to compositions and methods for predicting, preventing, and/or treating BE and/or EAC. The present disclosure is based, at least in part, on the discovery of three genes □ macrophage scavenger receptor 1 (MSR1), activating signal cointegrator 1 complex subunit 1 (ASCC1), and collagen triple-helix repeat-containing 1 (CTHRC1) □ which, when mutated in the germline, predispose to the development of BE and EAC. Additionally, the present disclosure is based in part on the discovery that certain germline mutations in MSR1 are significantly associated with the presence of BE and EAC, and that MSR1-mutation carriers show increased nuclear expression of Cyclin D1 (CCND1). EAC is often detected at later stages and is most often associated with poor prognosis. Advantageously, the inventor of the present disclosure has identified certain risk alleles that may predispose individuals to BE and/or EAC, thereby providing a means for premorbid risk assessment and help in better identifying treatment options.

### Compositions

Isolated Nucleic Acid Molecules

One aspect of the present disclosure pertains to isolated nucleic acid molecules that encode polypeptides or biologically active portions thereof, as well as nucleic acid fragments sufficient for use as hybridization probes to identify nucleic acids encoding the polypeptides, and fragments for use as PCR primers for the amplification or mutation of the nucleic acid molecules. In some instances, an isolated nucleic acid molecule of the present disclosure can comprise a risk allele associated with BE, EAC, or both. In other instances, an isolated nucleic acid molecule of the present disclosure can include one or more mutant genes associated with BE, EAC, or both, such as MSR1, ASCC1 and CTHRC1. In further instances, an isolated nucleic acid molecule of the present disclosure can include one or more genes associated with BE, EAC, or both (e.g., MSR1, ASCC1 and CTHRC1) having at least one germline mutation. In still further instances, an isolated nucleic acid molecule of the present disclosure can include one or more genes associated with BE, EAC, or both (e.g., MSR1, ASCC1 and CTHRC1) having at least one somatic mutation. Mutations that may be present in genes associated with BE, EAC, or both, can include, but are not limited to, point mutations (e.g., silent mutations, missense mutations, and nonsense mutations), insertions, deletions and frameshift mutations. It will be appreciated that a mutation (or mutations) in a nucleic acid molecule (e.g., somatic or germline DNA), such as MSR1, ASCC1, and/or CTHRC1 can include a change in a base (or bases) other than those in SEQ ID NOS: 7-8, 13, 17, 27-30 and 37, but still result in the amino acid change in SEQ ID NOS:9-10, 14, 18, 31-34 and 38.

In another aspect, an isolated nucleic acid molecule can comprise a human mutant MSR1 polynucleotide having SEQ ID NO:7 or SEQ ID NO:8. The human MSR1 gene and corresponding nucleotide sequences are known. See, e.g., M. Emi et al., J. Biol. Chem. 268(3), 2120-2125 (1993), A. Matsumoto et al., Proc. Natl. Acad. Sci. USA 87(23), 9133-9137 (1990), NCBI Accession Number P21757 (protein) and GenBank Accession Number D90187 (mRNA). MSR1 encodes three different types of isoforms generated by alternative splicing of the gene. As used herein, the term “MSR1” will typically refer to MSR1 isoform I; however, in some instances, the term can refer to isoform II, isoform III, or isoforms I-III. Human MSR1 consists of 11 exons. Two types of mRNAs are generated by alternative splicing from exon 8 to either exon 9 (isoform II) or to exons 10 and 11 (isoform I). Exon 1 encodes the 5′ untranslated region followed by a 12-kb intron, which separates the transcription initiation and the translation initiation sites. Exon 2 encodes a cytoplasmic domain, exon 3 encodes a transmembrane domain, exons 4 and 5 encode an α-helical coiled-coil, and exons 6-8 encode a collagen-like domain. MSR1 is assigned to 8p22. Thus, in some instances, an isolated nucleic acid molecule can comprise a human MSR1 polynucleotide having at least one germline missense mutation in exon 5 (e.g., a 760C>G mutation) (SEQ ID NO:8), which leads to a Leu245Val amino acid change. In other instances, an isolated nucleic acid molecule can comprise a human MSR1 polynucleotide having at least one germline nonsense mutation in exon 6 (e.g., a 877C>T mutation) (SEQ ID NO:7), which leads to a Arg293X amino acid change.

In further instances, an isolated nucleic acid can comprise a mutant human MSR1 polynucleotide having a nucleotide sequence that is at least about 60%, 65%, 70%, 75%, 80%, 85%, 90%, 95%, 98% or more homologous to the nucleotide sequence (e.g., to the entire length of the nucleotide sequence) shown in SEQ ID NO:7. In other instances, an isolated nucleic acid can comprise a mutant human MSR1 polynucleotide having a nucleotide sequence that is at least about 60%, 65%, 70%, 75%, 80%, 85%, 90%, 95%, 98% or more homologous to the nucleotide sequence (e.g., to the entire length of the nucleotide sequence) shown in SEQ ID NO:8. In some instances, an isolated nucleic acid molecule of the present disclosure can comprise a nucleic acid molecule that is a complement of the nucleotide sequence shown in SEQ ID NOS:7-8, or a portion of any of these nucleotide sequences. A nucleic acid molecule that is complementary to the nucleotide sequences shown in SEQ ID NOS:7-8 is one that is sufficiently complementary to the nucleotide sequences shown in SEQ ID NOS:7-8, such that it can hybridize to the nucleotide sequences shown in SEQ ID NOS:7-8 and thereby form a stable duplex.

It will be appreciated that isolated nucleic acid molecules of the present disclosure can additionally include polynucleotides that encode isoforms of MSR1, such as MSR1 isoform II (MSR1-II) and MSR1 isoform III (MSR1-III). Wild-type human polynucleotides that encode MSR1-II and MSR-III polypeptides are shown in SEQ ID NOS:2-3. Thus, in some instances, an isolated nucleic acid molecule can include a mutant MSR1 polynucleotide having SEQ ID NO:27 or SEQ ID NO:28. In other instances, an isolated nucleic acid molecule can include a mutant MSR1 polynucleotide having SEQ ID NO:29 or SEQ ID NO:30. In further instances, an isolated nucleic acid can comprise a mutant human MSR1-II polynucleotide having a nucleotide sequence that is at least about 60%, 65%, 70%, 75%, 80%, 85%, 90%, 95%, 98% or more homologous to the nucleotide sequences (e.g., to the entire length of the nucleotide sequence) shown in SEQ ID NO:27 or SEQ ID NO:28. In other instances, an isolated nucleic acid can comprise a mutant human MSR1 polynucleotide having a nucleotide sequence that is at least about 60%, 65%, 70%, 75%, 80%, 85%, 90%, 95%, 98% or more homologous to the nucleotide sequences (e.g., to the entire length of the nucleotide sequence) shown in SEQ ID NO:29 or SEQ ID NO:30. In still further instances, an isolated nucleic acid molecule of the present disclosure can comprise a nucleic acid molecule that is a complement of the nucleotide sequences shown in SEQ ID NOS:27-30, or a portion of any of these nucleotide sequences. A nucleic acid molecule that is complementary to the nucleotide sequences shown in SEQ ID NOS:27-30 is one that is sufficiently complementary to the nucleotide sequences shown in SEQ ID NOS:27-30, such that it can hybridize to the nucleotide sequences shown in SEQ ID NOS:27-30 and thereby form a stable duplex.

In another aspect, an isolated nucleic acid molecule of the present disclosure can comprise a mutant ASCC1 polynucleotide that includes at least one mutation (e.g., a germline mutation). ASCC1 encodes a subunit of the activating signal cointegrator 1 (ASC-1) complex. The ASC-1 complex is a transcriptional coactivator that plays an important role in gene transactivation by multiple transcription factors, including activating protein 1 (AP-1), nuclear factor kappa-B (NF-kB), and serum response factor (SRF). The encoded protein contains an N-terminal KH-type RNA-binding motif, which is required for AP-1 transactivation by the ASC-1 complex. Thus, in some instances, an isolated nucleic acid molecule can comprise a human ASCC1 polynucleotide having at least one germline missense mutation in exon 8 (e.g., a 869A>G mutation) (SEQ ID NO:13), which leads to an Asn290Ser amino acid change. As discussed in more detail below, it will be appreciated that an isolated nucleic acid molecule of the present disclosure can additionally include polynucleotides that have at least one mutation (e.g., a germline mutation) and encode an ASCC1 isoform.

In further instances, an isolated nucleic acid can comprise a mutant human ASCC1 polynucleotide having a nucleotide sequence that is at least about 60%, 65%, 70%, 75%, 80%, 85%, 90%, 95%, 98% or more homologous to the nucleotide sequence (e.g., to the entire length of the nucleotide sequence) shown in SEQ ID NO:13. In some instances, an isolated nucleic acid molecule of the present disclosure can comprise a nucleic acid molecule that is a complement of the nucleotide sequence shown in SEQ ID NO:13, or a portion of any of this nucleotide sequence. A nucleic acid molecule that is complementary to the nucleotide sequence shown in SEQ ID NO:13 is one that is sufficiently complementary to the nucleotide sequence shown in SEQ ID NO:13, such that it can hybridize to the nucleotide sequence shown in SEQ ID NO:13 and thereby form a stable duplex:

It will be appreciated that isolated nucleic acid molecules of the present disclosure can additionally include polynucleotides that encode isoforms of ASCC1, such as ASCC1 isoform b (ASCC1-b) (SEQ ID NO:36). The wild-type human polynucleotide that encodes the ASCC1-b polypeptide is shown in SEQ ID NO:35. Thus, in some instances, an isolated nucleic acid molecule can include a mutant ASCC1 polynucleotide having SEQ ID NO:37. In further instances, an isolated nucleic acid can comprise a mutant human ASCC1 polynucleotide having a polynucleotide sequence that is at least about 60%, 65%, 70%, 75%, 80%, 85%, 90%, 95%, 98% or more homologous to the nucleotide sequence (e.g., to the entire length of the nucleotide sequence) shown in SEQ ID NO:37. In other instances, an isolated nucleic acid molecule of the present disclosure can comprise a nucleic acid molecule that is a complement of the nucleotide sequence shown in SEQ ID NO:37, or a portion of any of this nucleotide sequence. A nucleic acid molecule that is complementary to the nucleotide sequence shown in SEQ ID NO:37 is one that is sufficiently complementary to the nucleotide sequence shown in SEQ ID NO:37, such that it can hybridize to the nucleotide sequence shown in SEQ ID NO:37 and thereby form a stable duplex.

In another aspect, an isolated nucleic acid molecule of the present disclosure can include a human CTHRC1 polynucleotide that includes at least one mutation (e.g., a germline mutation). CTHRC1 encodes a 28-30 kDa secreted glycoprotein that bears similarity to the Clq/TNFα-related family of proteins. It is expressed by disparate cell types, including renal epithelium, neurons, osteoblasts, and smooth muscle cells. Functionally, it is recognized to be induced by BMP-2 and to block TGF β-induced collagen type I and III synthesis. Proteolytic processing may generate multiple CTHRC1 isoforms. CTHRC1 may undergo dimerization, trimerization and oligomerization. Thus, in some instances, an isolated nucleic acid molecule can comprise a human CTHRC1 polynucleotide having at least one germline missense mutation in exon 1 (e.g., a 131A>C mutation) (SEQ ID NO:17), which leads to a Gln44Pro amino acid change. It will be appreciated that an isolated nucleic acid molecule of the present disclosure can additionally include polynucleotides that have one or more mutations and encode a CTHRC1 isoform.

In further instances, an isolated nucleic acid can comprise a mutant human CTHRC1 polynucleotide having a nucleotide sequence that is at least about 60%, 65%, 70%, 75%, 80%, 85%, 90%, 95%, 98% or more homologous to the nucleotide sequence (e.g., to the entire length of the nucleotide sequence) shown in SEQ ID NO:17. In some instances, an isolated nucleic acid molecule of the present disclosure can comprise a nucleic acid molecule that is a complement of the nucleotide sequence shown in SEQ ID NO:17, or a portion of any of this nucleotide sequence. A nucleic acid molecule that is complementary to the nucleotide sequence shown in SEQ ID NO:17 is one that is sufficiently complementary to the nucleotide sequence shown in SEQ ID NO:17, such that it can hybridize to the nucleotide sequence shown in SEQ ID NO:17 and thereby form a stable duplex.

A nucleic acid molecule of the present disclosure, e.g., a nucleic acid molecule having the nucleotide sequence of SEQ ID NOS:7-8, 13, 17, 27-30 or 37, or a portion thereof, can be isolated using standard molecular biology techniques and the sequence information provided herein. For example, using all or portion of the nucleic acid sequence of SEQ ID NOS:7-8, 13, 17, 27-30, or 37 as a hybridization probe, the nucleic acid molecules can be isolated using standard hybridization and cloning techniques (e.g., as described in Sambrook, J., Fritsh, E. F., and Maniatis, T. Molecular Cloning. A Laboratory Manual. 2nd, ed., Cold Spring Harbor Laboratory, Cold Spring Harbor Laboratory Press, Cold Spring Harbor, N.Y., 1989).

Moreover, a nucleic acid molecule encompassing all or a portion of SEQ ID NOS: 7-8, 13, 17, 27-30, or 37 can be isolated by the polymerase chain reaction (PCR) using synthetic oligonucleotide primers designed based upon the sequence of SEQ ID NOS: 7-8, 13, 17, 27-30 or 37, respectively. A nucleic acid molecule of the present disclosure can be amplified using cDNA, mRNA, or alternatively, genomic DNA as a template and appropriate oligonucleotide primers according to standard PCR amplification techniques. The nucleic acid so amplified can be cloned into an appropriate vector and characterized by DNA sequence analysis. Furthermore, oligonucleotides corresponding to the nucleotide sequences of the present disclosure can be prepared by standard synthetic techniques, e.g., using an automated DNA synthesizer.

The present disclosure further encompasses nucleic acid molecules that differ from the nucleotide sequence shown in SEQ ID NOS: 7-8, 13, 17, 27-30, and 37 due to the degeneracy of the genetic code and, thus, encode the same polypeptides as those encoded by the nucleotide sequences shown in SEQ ID NOS: 7-8, 13, 17, 27-30 and 37 (discussed below).

It will be appreciated that isolated nucleic acid molecules of the present disclosure can also include all or only a fragment of those listed in Tables 1-2 and Tables 4-5.

Isolated Polypeptides and Antibodies

Another aspect of the present disclosure pertains to isolated polypeptides, proteins and biologically active portions thereof, as well as polypeptide fragments suitable for use as immunogens to raise antibodies. It will be appreciated that the terms “polypeptide” and “protein” can be used interchangeably herein. In some instances, an isolated polypeptide Molecule can comprise a mutant human MSR1, ASCC1, or CTHRC1 polypeptide that is associated with BE, EAC, or both (e.g., the isolated polypeptide molecule(s) predispose to the development of BE and/or EAC). In other instances, an isolated polypeptide molecule of the present disclosure can comprise a mutant human MSR1 polypeptide having the amino acid sequence of SEQ ID NO:9 or SEQ ID NO:10. In further instances, an isolated polypeptide can comprise a mutant human MSR1 polypeptide having an amino acid sequence that is at least about 60%, 65%, 70%, 75%, 80%, 85%, 90%, 95%, 98% or more homologous to the amino acid sequence (e.g., to the entire length of the amino acid sequence) shown in SEQ ID NO:9. In still further instances, an isolated polypeptide can comprise a mutant human MSR1 polypeptide having an amino acid sequence that is at least about 60%, 65%, 70%, 75%, 80%, 85%, 90%, 95%, 98% or more homologous to the amino acid sequence (e.g., to the entire length of the amino acid sequence) shown in SEQ ID NO:10.

In another aspect, an isolated polypeptide molecule of the present disclosure can comprise a mutant human MSR1 polypeptide having an amino acid sequence of SEQ ID NOS:31-32. In some instances, an isolated polypeptide can comprise a mutant human MSR1 polypeptide having an amino acid sequence that is at least about 60%, 65%, 70%, 75%, 80%, 85%, 90%, 95%, 98% or more homologous to the amino acid sequences (e.g., to the entire length of the amino acid sequence) shown in SEQ ID NOS:31-32.

In another aspect, an isolated polypeptide molecule of the present disclosure can comprise a mutant human MSR1 polypeptide having an amino acid sequence of SEQ ID NOS:33-34. In some instances, an isolated polypeptide can comprise a mutant human MSR1 polypeptide having an amino acid sequence that is at least about 60%, 65%, 70%, 75%, 80%, 85%, 90%, 95%, 98% or more homologous to the amino acid sequences (e.g., to the entire length of the amino acid sequence) shown in SEQ ID NOS:33-34.

In another aspect, an isolated polypeptide molecule of the present disclosure can comprise a mutant human ASCC1 polypeptide including ASCC1 isoform a or ASCC1 isoform b. In some instances, an isolated polypeptide molecule can comprise a mutant human ASCC1 polypeptide (isoform a) having an amino acid sequence of SEQ ID NO:14. In other instances, an isolated polypeptide molecule can comprise a mutant human ASCC1 polypeptide (isoform b) having an amino acid sequence of SEQ ID NO:38. In other instances, an isolated polypeptide can comprise a mutant human ASCC1 polypeptide (isoform a or b) having an amino acid sequence that is at least about 60%, 65%, 70%, 75%, 80%, 85%, 90%, 95%, 98% or more homologous to the amino acid sequences (e.g., to the entire length of the amino acid sequence) shown in SEQ ID NO:14 or SEQ ID NO:38.

In another aspect, an isolated polypeptide molecule of the present disclosure can comprise a mutant human CTHRC1 polypeptide having an amino acid sequence of SEQ ID NO:18. In some instances, an isolated polypeptide can comprise a mutant human CTHRC1 polypeptide having an amino acid sequence that is at least about 60%, 65%, 70%, 75%, 80%, 85%, 90%, 95%, 98% or more homologous to the amino acid sequence (e.g., to the entire length of the amino acid sequence) shown in SEQ ID NO:18.

It will be appreciated that isolated polypeptide molecules of the present disclosure can also include all or only a fragment of the polypeptides encoded by the genes listed in Tables 1-2 and Tables 4-5.

In another aspect, proteins encoded by the nucleic acids of the present disclosure can be isolated from cells or tissue sources by an appropriate purification scheme using standard protein purification techniques. In some instances, proteins of the present disclosure can be produced by recombinant DNA techniques. Alternative to recombinant expression, a protein or polypeptide can be synthesized chemically using standard peptide synthesis techniques.

An “isolated” or “purified” protein, polypeptide, or biologically active portion thereof can be substantially free of cellular material or other contaminating proteins from the cell or tissue source from which the polypeptide or protein is derived, or substantially free from chemical precursors or other chemicals when chemically synthesized. The term “substantially free of cellular material” can include preparations of proteins in which the protein is separated from cellular components of the cells from which it is isolated or recombinantly produced.

In some instances, the term “substantially free of cellular material” can include preparations of a protein encoded by a nucleic acid molecule of the present disclosure (e.g., SEQ ID NOS:7-8, 13, 17, 27-30 and 37) having less than about 30% (by dry weight) of proteins other than the protein being purified or isolated (also referred to herein as a “contaminating protein”), less than about 20% of contaminating protein, less than about 10% of contaminating protein, or less than about 5% contaminating protein. When the protein, or biologically active portion thereof, is recombinantly produced, it can also be substantially free of culture medium, i.e., culture medium represents less than about 20%, less than about 10%, or less than about 5% of the volume of the protein preparation.

The term “substantially free of chemical precursors or other chemicals” can include preparations of a protein or polypeptide of the present disclosure in which the protein is separated from chemical precursors or other chemicals which are involved in the synthesis of the protein. In some instances, the term “substantially free of chemical precursors or other chemicals” can include preparations of a protein of the present disclosure having less than about 30% (by dry weight) of chemical precursors, less than about 20% chemical precursors, less than about 10% chemical precursors, or less than about 5% chemical precursors.

Biologically active portions of a protein of the present disclosure can include peptides comprising amino acid sequences sufficiently homologous to or derived from the amino acid sequence of the polypeptide, which include fewer amino acids than the full length proteins, and exhibit at least one activity of a protein. Typically, biologically active portions can comprise a domain or motif with at least one activity of the protein or polypeptide. For example, a biologically active portion can be a polypeptide which is, for example, at least 10, 25, 50, 100 or more amino acids in length. Such fragments may be linear or in a cyclized form using methods know in the art, such as those found in H. U. Saragovi, et al., BioTechnology 10, 773-778 (1992) and R. S. McDowell, et al., J. Amer. Chem. Soc. 114, 9245-9253 (1992).

Where the polypeptides of the present disclosure are found to be membrane bound entities, the present disclosure also provides for their soluble form. In these instances, the membrane bound portions of the polypeptides can be deleted using standard methods so that they are secreted from the cell upon expression. Sequence information can be used by those knowledgeable in the art to determine where the membrane bound regions are located within the polypeptide sequence.

To determine the percent identity of two amino acid sequences or of two nucleic acid sequences, the sequences can be aligned for optimal comparison purposes (e.g., gaps can be introduced in one or both of a first and a second amino acid or nucleic acid sequence for optimal alignment and non-homologous sequences can be disregarded for comparison purposes). In some instances, the length of a reference sequence aligned for comparison purposes can be at least 30%, at least 40%, at least 50%, at least 60%, or at least 70%, 80%, or 90% of the length of the reference sequence. The amino acid residues or nucleotides at corresponding amino acid positions (or nucleotide positions) can then be compared. When a position in the first sequence is occupied by the same amino acid residue or nucleotide as the corresponding position in the second sequence, then the molecules are identical at that position (as used herein, amino acid or nucleic acid “identity” can be equivalent to amino acid or nucleic acid “homology”). The percent identity between the two sequences is a function of the number of identical positions shared by the sequences, taking into account the number of gaps and the length of each gap, which need to be introduced for optimal alignment of the two sequences. The comparison of sequences and determination of percent identity between two sequences can be accomplished using a mathematical algorithm known in the art.

The present disclosure also provides chimeric or fusion proteins. As used herein, a “chimeric protein” or “fusion protein” can comprise a polypeptide of the present disclosure (e.g., encoded by a nucleic acid having a sequence shown in SEQ ID NOS:7-8, 13, 17 27-30 or 37, or having an amino acid sequence shown in SEQ ID NOS:9-10, 14, 18, 31-34 or 38) operatively linked to a heterologous polypeptide. A “heterologous polypeptide” can refer to a polypeptide having an amino acid sequence corresponding to a protein that is not substantially homologous to the polypeptide of the present disclosure used in the chimeric or fusion protein (e.g., a protein which is different from the polypeptide of the invention and which is derived from the same or a different organism). The fusion protein can contain all or a portion of a polypeptide of the present disclosure. In some instances, a fusion protein can comprise at least one biologically active portion of a protein of the present disclosure. In other instances, a fusion protein comprises at least two biologically active portions of a protein of the present disclosure. Within the fusion protein, the term “operatively linked” can indicate that the polypeptide of the present disclosure and the heterologous polypeptide are fused in-frame to each other. In some instances, the heterologous polypeptide can be fused to the N-terminus or C-terminus of the polypeptide of the present disclosure.

A chimeric or fusion protein of the present disclosure can be produced by standard recombinant DNA techniques. For example, DNA fragments coding for the different polypeptide sequences can be ligated together in-frame in accordance with conventional techniques, for example, by employing blunt-ended or stagger-ended termini for ligation, restriction enzyme digestion to provide for appropriate termini, filling-in of cohesive ends as appropriate, alkaline phosphatase treatment to avoid undesirable joining, and enzymatic ligation. In some instances, the fusion gene can be synthesized by conventional techniques, such as automated DNA synthesizers. Alternatively, PCR amplification of gene fragments can be carried out using anchor primers, which give rise to complementary overhangs between two consecutive gene fragments that can subsequently be annealed and reamplified to generate a chimeric gene sequence (see, for example, Current Protocols in Molecular Biology, eds. Ausubel et al. John Wiley & Sons: 1992). Moreover, many expression vectors are commercially available that already encode a fusion moiety (e.g., a GST polypeptide). A nucleic acid encoding a polypeptide of the present disclosure can be cloned into such an expression vector so that the fusion moiety is linked in-frame to the polypeptide.

In another aspect of the present disclosure, an isolated polypeptide of the present disclosure (e.g., having an amino acid sequence of SEQ ID NOS:9-10, 14, 18, 31-34 or 38), or a portion or fragment thereof, can be used as an immunogen to generate antibodies that bind to the polypeptide using standard techniques for polyclonal and monoclonal antibody preparation. Thus, in some instances, the present disclosure can include an isolated antibody that specifically binds to a polypeptide molecule having an amino acid sequence of SEQ ID NOS:9-10, 14, 18, 31-34 or 38. As explained in more detail below, isolated antibodies of the present disclosure can be prepared as described in U.S. Pat. No. 7,348,414.

A full-length protein can be used or, alternatively, the present disclosure provides antigenic peptide fragments of the polypeptides for use as immunogens. An antigenic peptide of a polypeptide of the present disclosure can comprise at least about 8 amino acid residues and encompasses an epitope of polypeptide so that an antibody raised against the peptide forms a specific immune complex with the polypeptide. In some instances, the antigenic peptide comprises at least about 10 amino acid residues, at least about 15 amino acid residues, at least about 20 amino acid residues, or at least about 30 amino acid residues. Epitopes encompassed by the antigenic peptide can include regions of the polypeptide that are located on the surface of the protein, e.g., hydrophilic regions.

A polypeptide immunogen is typically used to prepare antibodies by immunizing a suitable subject (e.g., rabbit, goat, mouse or other mammal) with the immunogen. An appropriate immunogenic preparation can contain, for example, recombinantly expressed or a chemically synthesized polypeptide. The preparation can further include an adjuvant, such as Freund's complete or incomplete adjuvant, or similar immunostimulatory agent. Immunization of a suitable subject with an immunogenic polypeptide preparation can induce a polyclonal antibody response to the polypeptide.

Accordingly, another aspect of the present disclosure can include antibodies that bind to polypeptides of the present disclosure (e.g., a polypeptide encoded by a nucleic acid having the sequence of SEQ ID NOS:7-8, 13, 17, 27-30 or 37, or having the amino acid sequence of SEQ ID NOS:9-10, 14, 18, 31-34 or 38). The term “antibody” as used herein can refer to immunoglobulin molecules and immunologically active portions of immunoglobulin molecules, i.e., molecules that contain an antigen binding site that specifically binds (immunoreacts with) an antigen, e.g., a polypeptide of the present disclosure. Examples of immunologically active portions of immunoglobulin molecules can include F(ab) and F(ab′)₂ fragments, which can be generated by treating the antibody with an enzyme, such as pepsin. The present disclosure provides polyclonal and monoclonal antibodies that bind to the polypeptides of the present disclosure. The term “monoclonal antibody” or “monoclonal antibody composition”, as used herein, can refer to a population of antibody molecules that contain only one species of an antigen binding site capable of immunoreacting with a particular epitope of a polypeptide. A monoclonal antibody composition thus typically displays a single binding affinity for a particular protein with which it immunoreacts.

Polyclonal antibodies that bind to a polypeptide of the present disclosure can be prepared as described above (e.g., by immunizing a suitable subject with a polypeptide immunogen). The antibody titer in the immunized subject can be monitored over time by standard techniques, such as with an enzyme linked immunosorbent assay (ELISA) using immobilized immunogen. If desired, the antibody molecules directed against a polypeptide of the present disclosure can be isolated from the mammal (e.g., from the blood) and further purified by well known techniques, such as protein A chromatography to obtain the IgG fraction. At an appropriate time after immunization, e.g., when the antibody titers are highest, antibody-producing cells can be obtained from the subject and used to prepare monoclonal antibodies by standard techniques, such as the hybridoma technique originally described by Kohler and Milstein (1975) Nature 256:495-497) (see also, Brown et al. (1981) J. Immunol. 127:539-46; Brown et al. (1980) J. Biol. Chem 255:4980-83; Yeh et al. (1976) Proc. Natl. Acad. Sci. USA 76:2927-31; and Yeh et al. (1982) Int. J. Cancer 29:269-75), the more recent human B cell hybridoma technique (Kozbor et al. (1983) Immunol Today 4:72), the EBV-hybridoma technique (Cole et al. (1985), Monoclonal Antibodies and Cancer Therapy, Alan R. Liss, Inc., pp. 77-96) or trioma techniques. The technology for producing monoclonal antibody hybridomas is well known (see generally R. H. Kenneth, in Monoclonal Antibodies: A New Dimension In Biological Analyses, Plenum Publishing Corp., New York, N.Y. (1980); E. A. Lerner (1981) Yale J. Biol. Med., 54:387-402; M. L. Gefter et al. (1977) Somatic Cell Genet. 3:231-36). Briefly, an immortal cell line (typically a myeloma) is fused to lymphocytes (typically splenocytes) from a mammal immunized with a polypeptide immunogen of the invention as described above, and the culture supernatants of the resulting hybridoma cells are screened to identify a hybridoma producing a monoclonal antibody that binds to the polypeptide.

Any of the many well known protocols used for fusing lymphocytes and immortalized cell lines can be applied for the purpose of generating an a monoclonal antibody specific for a polypeptide of the present disclosure (see, e.g., G. Galfre et al. (1977) Nature 266:55052; Gefter et al. Somatic Cell Genet., cited supra; Lerner, Yale J. Biol. Med., cited supra; Kenneth, Monoclonal Antibodies, cited supra). Moreover, the skilled artisan will appreciate that there are many variations of such methods that would also be useful. Typically, the immortal cell line (e.g., a myeloma cell line) is derived from the same mammalian species as the lymphocytes. For example, murine hybridomas can be made by fusing lymphocytes from a mouse immunized with an immunogenic preparation of the present disclosure with an immortalized mouse cell line. Examples of immortal cell lines are mouse myeloma cell lines that are sensitive to culture medium containing hypoxanthine, aminopterin and thymidine (“HAT medium”).

Any of a number of myeloma cell lines can be used as a fusion partner according to standard techniques, e.g., the P3-NS1/1-Ag-4-1, P3-x63-Ag8.653 or Sp2/O—Ag14 myeloma lines. These myeloma lines are available from ATCC. Typically, HAT-sensitive mouse myeloma cells are fused to mouse splenocytes using polyethylene glycol (“PEG”). Hybridoma cells resulting from the fusion are then selected using HAT medium, which kills unfused and unproductively fused myeloma cells (unfused splenocytes die after several days because they are not transformed). Hybridoma cells producing a monoclonal antibody of the present disclosure are detected by screening the hybridoma culture supernatants for antibodies that bind to the polypeptide(s) of the present disclosure (e.g., using a standard ELISA assay).

Alternative to preparing monoclonal antibody-secreting hybridomas, a monoclonal antibody for a polypeptide of the present disclosure can be identified and isolated by screening a recombinant combinatorial immunoglobulin library (e.g., an antibody phage display library) with the polypeptide to thereby isolate immunoglobulin library members that bind to the polypeptide. Kits for generating and screening phage display libraries are commercially available (e.g., the Pharmacia Recombinant Phage Antibody System, Catalog No. 27-9400-01; and the Stratagene SurfZAP™ Phage Display Kit, Catalog No. 240612). Additionally, examples of methods and reagents particularly amenable for use in generating and screening antibody display library can be found in, for example, Ladner et al. U.S. Pat. No. 5,223,409; Kang et al. PCT International Publication No. WO 92/18619; Dower et al. PCT International Publication No. WO 91/17271; Winter et al. PCT International Publication WO 92/20791; Markland et al. PCT International Publication No. WO 92/15679; Breitling et al. PCT International Publication WO 93/01288; McCafferty et al. PCT International Publication No. WO 92/01047; Garrard et al. PCT International Publication No. WO 92/09690; Ladner et al. PCT International Publication No. WO 90/02809; Fuchs et al. (1991) Bio/Technology 9:1370-1372; Hay et al. (1992) Hum. Antibod. Hybridomas 3:81-85; Huse et al. (1989) Science 246:1275-1281; Griffiths et al. (1993) EMBO J 12:725-734; Hawkins et al. (1992) J. Mol. Biol. 226:889-896; Clarkson et al. (1991) Nature 352:624-628; Gram et al. (1992) Proc. Natl. Acad. Sci. USA 89:3576-3580; Garrad et al. (1991) Bio/Technology 9:1373-1377; Hoogenboom et al. (1991) Nuc. Acid Res. 19:4133-4137; Barbas et al. (1991) Proc. Natl. Acad. Sci. USA 88:7978-7982; and McCafferty et al. Nature (1990) 348:552-554.

Additionally, recombinant antibodies, such as chimeric and humanized monoclonal antibodies, comprising both human and non-human portions, which can be made using standard recombinant DNA techniques, are within the scope of the present disclosure. Such chimeric and humanized monoclonal antibodies can be produced by recombinant DNA techniques known in the art, for example, using methods described in Robinson et al. International Application No. PCT/US86/02269; Akira, et al. European Patent Application 184,187; Taniguchi, M., European Patent Application 171,496; Morrison et al. European Patent Application 173,494; Neuberger et al. PCT International Publication No. WO 86/01533; Cabilly et al. U.S. Pat. No. 4,816,567; Cabilly et al. European Patent Application 125,023; Better et al. (1988) Science 240:1041-1043; Liu et al. (1987) Proc. Natl. Acad. Sci. USA 84:3439-3443; Liu et al. (1987) J. Immunol. 139:3521-3526; Sun et al. (1987) Proc. Natl. Acad. Sci. USA 84:214-218; Nishimura et al. (1987) Canc. Res. 47:999-1005; Wood et al. (1985) Nature 314:446-449; and Shaw et al. (1988) J. Natl. Cancer Inst. 80:1553-1559); Morrison, S. L. (1985) Science 229:1202-1207; Oi et al. (1986) BioTechniques 4:214; Winter U.S. Pat. No. 5,225,539; Jones et al. (1986) Nature 321:552-525; Verhoeyan et al. (1988) Science 239:1534; and Beidler et al. (1988) J. Immunol. 141:4053-4060.

An antibody (e.g., monoclonal antibody) specific for a polypeptide of the present disclosure can be used to isolate the polypeptide by standard techniques, such as affinity chromatography or immunoprecipitation. The antibody can facilitate the purification of natural polypeptide from cells and of recombinantly produced polypeptide expressed in host cells. Moreover, an antibody specific for a polypeptide of the invention can be used to detect the polypeptide (e.g., in a cellular lysate or cell supernatant) in order to evaluate the abundance and pattern of expression of the protein. Antibodies can further be used diagnostically to monitor protein levels in tissue as part of a clinical testing procedure, e.g., to determine the efficacy of a given treatment regimen. Detection can be facilitated by coupling (i.e., physically linking) the antibody to a detectable substance. Examples of detectable substances can include various enzymes, prosthetic groups, fluorescent materials, luminescent materials, bioluminescent materials, and radioactive materials. Examples of suitable enzymes can include horseradish peroxidase, alkaline phosphatase, β-galactosidase, or acetylcholinesterase. Examples of suitable prosthetic group complexes include streptavidin/biotin and avidin/biotin. Examples of suitable fluorescent materials include umbelliferone, fluorescein, fluorescein isothiocyanate, rhodamine, dichlorotriazinylamine fluorescein, dansyl chloride or phycoerythrin. An example of a luminescent material includes luminal. Examples of bioluminescent materials include luciferase, luciferin, and aequorin. Examples of suitable radioactive material include ¹²⁵I, ¹³¹I, ³⁵S or ³H.

Computer Readable Media

In another aspect, the nucleotide or amino acid sequences of the present disclosure can be provided in a variety of media to facilitate use thereof. As used herein, “provided” can refer to a manufacture, other than an isolated nucleic acid or amino acid molecule, which contains a nucleotide or amino acid sequences of the present disclosure. Such a manufacture can provide the nucleotide or amino acid sequences, or a subset thereof (e.g., a subset of open reading frames) in a form, which allows a skilled artisan to examine the manufacture using means not directly applicable to examining the nucleotide or amino acid sequences, or a subset thereof, as they exist in nature or in purified form.

In some instances, a nucleotide or amino acid sequence of the present disclosure can be recorded on computer readable media. As used herein, the term “computer readable media” can include any medium that can be read and accessed directly by a computer. Such media can include, but are not limited to: magnetic storage media, such as floppy discs, hard disc storage medium, and magnetic tape; optical storage media such a CD-ROM; electrical storage media, such as RAM and ROM; and hybrids of these categories, such as magnetic/optical storage media. The skilled artisan will readily appreciate how any of the presently known computer readable media can be used to create a manufacture comprising computer readable media having recorded thereon a nucleotide or amino acid sequence of the present disclosure.

As used herein, the term “recorded” can refer to a process of storing information on a computer readable medium. The skilled artisan can readily adopt any of the presently known methods for recording information on a computer readable medium to generate manufactures comprising the nucleotide or amino acid sequence information of the present disclosure.

A variety of data storage structures are available to a skilled artisan for creating a computer readable medium having recorded thereon a nucleotide or amino acid sequence of the present disclosure. The choice of the data storage structure will generally be based on the means chosen to access the stored information. In addition, a variety of data processor programs and formats can be used to store the nucleotide sequence information of the present disclosure on computer readable medium. The sequence information can be represented in a word processing text file, formatted in commercially-available software, such as WordPerfect and Microsoft Word, or represented in the form of an ASCHII file, stored in a database application, such as DB2, Sybase Oracle, or the like. The skilled artisan can readily adapt any number of data processor structuring formats (e.g., text file or database) in order to obtain computer readable media having recorded thereon the nucleotide sequence information of the present disclosure.

By providing the nucleotide or amino acid sequences of the present disclosure in computer readable form, the skilled artisan can routinely access the sequence information for a variety of purposes. For example, the skilled in the art can use the nucleotide or amino acid sequences of the present disclosure in computer readable form to compare a target sequence or target structural motif with the sequence information stored within the data storage means. Search means can additionally or alternatively be used to identity fragments or regions of the sequences of the present disclosure, which match a particular target sequence or target motif.

### Methods

In another aspect of the present disclosure, methods are provided for assessing risk, preventing, and/or treating a subject having, or suspected of having, a precursor lesion (e.g., BE), an esophageal cancer (e.g., EAC), or both. The methods of the present disclosure can include detection and/or use of one or more nucleic acid molecules (e.g., having a polynucleotide sequence of SEQ ID NOS:7-8, 13, 17, 27-30 and 37) and/or polypeptide molecules (e.g., having an amino acid sequence of SEQ ID NOS:9-10, 14, 18, 31-34 and 38). In some instances, detection of a nucleic acid molecule and/or polypeptide molecule of the present disclosure can serve as a basis for preventing progression of BE to EAC. In other instances, the methods of the present disclosure can be used to not only predict a subject's risk of BE and/or EAC, but also the family members of the subject. It will be appreciated that nucleic acid molecules and/or polypeptide molecules that may be find application in the methods of the present disclosure can include not only those discussed above, but also those listed in Tables 1-2 and Tables 4-5.

In some instances, the present disclosure can include a method for predicting a subject's risk of developing a precursor lesion (e.g., BE), an esophageal cancer (e.g., EAC), or both. The method can include the steps of: obtaining a biological sample from a subject; determining in the biological sample the presence of at least one germline mutation; and determining that the subject is at an increased risk of an esophageal cancer, a precursor lesion, or both, due to the presence of the at least one germline mutation.

In general, a subject to be evaluated by a method of the present disclosure can have, or may be suspected of having, a precursor lesion, an esophageal cancer, or both. Human subjects suspected of having BE, for example, often present with heartburn and are subjected to an endoscopy and biopsies to definitively determine whether they have BE and/or dysplasia. Thus, in some instances, a subject to be evaluated by the method(s) of the present disclosure can be a subject who reports (or is diagnosed with) abnormal acid reflux, but whom does not exhibit any other characteristics and/or symptoms of BE and/or EAC. Subjects who evince BE, with or without dysplasia, are generally monitored endoscopically periodically, even if symptoms later disappear. Subjects that can be evaluated by a method of the present disclosure can include any of a variety of vertebrates, including, e.g., laboratory animals (e.g., mouse, rat, rabbit, monkey, or guinea pig, in particular mouse or rat models for EAC), farm animals (e.g., cattle, horses, pigs, sheep, goats, etc.), and domestic animals or pets (e.g., cats or dogs). Non-human primates, such as humans are also included.

One step of the method can include obtaining one or more biological samples from a subject having, or being suspected of having, a precursor lesion, an esophageal cancer, or both. Any biological sample that contains the DNA of the subject may be employed, including tissue samples and blood samples. In one example, the biological sample can be a source of germline DNA (e.g., a germ cell). In another example, the biological sample can be a source of somatic DNA. Biological samples may be obtained using any of a number of methods in the art. For example, biological samples comprising esophageal cells can include those obtained from biopsies, cytologic specimens, and resected specimens. A cytologic specimen may be an endoscopic brushing specimen or a balloon cytology specimen. A biological specimen may also be embedded in paraffin and sectioned for use in the methods of the present disclosure.

Typically, biological samples, once obtained, can be harvested and processed prior to molecular analysis using standard methods known in the art. Such processing can include fixation in, for example, an acid alcohol solution, acid acetone solution, or aldehyde solution (e.g., formaldehyde and glutaraldehyde). Cells may then be concentrated to a desired density prior to analysis. Suitable samples (e.g., test samples, or control samples) that can be tested by a method of the present disclosure can include, e.g., biopsies of esophageal epithelium. For example, the sample can be from grossly apparent BE epithelium or from mass lesions in subjects manifesting these changes at endoscopic examination. Methods for obtaining samples and preparing them for analysis are conventional and well-known in the art.

After obtaining and preparing at least one biological sample, the presence of at least one gene mutation associated with a precursor lesion (e.g., BE), an esophageal cancer (e.g., EAC), or both, can be detected using one or more standard techniques. In some instances, the presence of at least one germline DNA mutation associated with a precursor lesion (e.g., BE), an esophageal cancer (e.g., EAC), or both, can be detected. For example, the presence of a risk allele having a polynucleotide sequence of SEQ ID NOS: 7-8, SEQ ID NO:13, SEQ ID NO:17, SEQ ID NOS:27-30 or SEQ ID NO:37 can be detected using, for example, probes, restriction enzyme digestion techniques, or other means of detecting the risk allele may be implemented based on corresponding known sequences (e.g., SEQ ID NOS:1-3,11, 15 and 35) in accordance with standard techniques. See, e.g., U.S. Pat. Nos. 6,027,896 and 5,767,248 to A. Roses et al. In some instances, determining the presence or absence of DNA containing a polymorphism or mutation of interest may be carried out with an oligonucleotide probe labeled with a suitable detectable group, or by means of an amplification reaction, such as PCR or ligase chain reaction (the product of which amplification reaction may then be detected with a labeled oligonucleotide probe or a number of other techniques). In other instances, the detecting step may include the step of detecting whether the subject is heterozygous or homozygous for the polymorphism or mutation of interest.

Probes and conditions can be selected using routine conventional procedures to insure that hybridization of a probe to a sequence of interest is specific. A probe that is “specific for” a nucleic acid sequence (e.g., in a DNA molecule) can contain sequences that are substantially similar to (e.g., hybridize under conditions of high stringency to) sequences in one of the strands of the nucleic acid. By hybridizing “specifically”, it is meant that the two components (the target DNA and the probe) can bind selectively to each other and not generally to other components unintended for binding to the subject components. The parameters required to achieve specific binding can be determined routinely, using conventional methods in the art. A probe that binds (hybridizes) specifically to a target of interest does not necessarily have to be completely complementary to it. For example, a probe can be at least about 95% identical to the target, provided that the probe binds specifically to the target under defined hybridization conditions, such conditions of high stringency.

Amplification of a selected, or target, nucleic acid sequence may be carried out by any suitable means. See generally D. Kwoh and T. Kwoh, Am. Biotechnol. Lab. 8, 14-25 (1990). Examples of suitable amplification techniques include, but are not limited to, PCR, ligase chain reaction, strand displacement amplification (see generally G. Walker et al., Proc. Natl. Acad. Sci. USA 89, 392-396 (1992); G. Walker et al., Nucleic Acids Res. 20, 1691-1696 (1992)), transcription-based amplification (see D. Kwoh et al., Proc. Natl. Acad Sci. USA 86, 1173-1177 (1989)), self-sustained sequence replication (or “3SR”) (see J. Guatelli et al., Proc. Natl. Acad. Sci. USA 87, 1874-1878 (1990)), the QB replicase system (see P. Lizardi et al., BioTechnology 6, 1197-1202 (1988)), nucleic acid sequence-based amplification (or “NASBA”) (see R. Lewis, Genetic Engineering News 12 (9), 1 (1992)), the repair chain reaction (or “RCR”) (see R. Lewis, supra), and boomerang DNA amplification (or “BDA”) (see R. Lewis, supra).

DNA amplification techniques, such as the foregoing can involve the use of a probe, a pair of probes, or two pairs of probes that specifically bind to DNA containing the polymorphism or gene mutation of interest, but do not bind to DNA that does not contain the polymorphism or gene mutation of interest under the same hybridization conditions, and which serve as the primer or primers for the amplification of the DNA or a portion thereof in the amplification reaction. Such probes are sometimes referred to as amplification probes or primers herein.

Probes and primers, including those for either amplification and/or protection, can include nucleotides (including naturally occurring nucleotides such as DNA and synthetic and/or modified nucleotides) of any suitable length, such as from 5, 6, or 8 nucleotides in length up to 40, 50 or 60 nucleotides in length, or more. Such probes and/or primers may be immobilized on or coupled to a solid support, such as a bead, chip, pin, or microtiter plate in accordance with known techniques, and/or coupled to or labeled with a detectable group, such as a fluorescent compound, a chemiluminescent compound, a radioactive element, or an enzyme in accordance with known techniques.

In one example, the presence or absence of at least one germline mutation in MSR1 can be detected using PCR. In some instances, PCR can be performed using oligonucleotide primers (e.g., having SEQ ID NOS:19-20) to detect the presence of at least one germline mutation in MSR1 (e.g., having a polynucleotide sequence of SEQ ID NO:8, SEQ ID NO:28 or SEQ ID NO:30). In other instances, PCR can be performed using oligonucleotide primers (e.g., having SEQ ID NOS:21-22) to detect the presence of at least one germline mutation in MSR1 (e.g., having a polynucleotide sequence of SEQ ID NO:7, SEQ ID NO:27 or SEQ ID NO:29). The presence or absence of a germline mutation in MSR1 can be determined by comparing the polynucleotide sequence obtained from the biological sample with a polynucleotide sequence obtained from a control (i.e., a wild-type sequence). Control or wild-type MSR1 polynucleotide sequences are shown in SEQ ID NOS:1-3.

In another example, the presence or absence of at least one germline mutation in ASCC1 can be detected using PCR. In some instances, PCR can be performed using oligonucleotide primers (e.g., having SEQ ID NOS:23-24) to detect the presence of at least one germline mutation in ASCC1 (e.g., having a polynucleotide sequence of SEQ ID NO:13). In other instances, PCR can be performed using oligonucleotide primers (e.g., having SEQ ID NOS:23-24) to detect the presence of at least one germline mutation in ASCC1 (e.g., having a polynucleotide sequence of SEQ ID NO:37). The presence or absence of a germline mutation in ASCC1 can be determined by comparing the polynucleotide sequence obtained from the biological sample with a polynucleotide sequence obtained from a control (i.e., a wild-type sequence). Control or wild-type ASCC1 polynucleotide sequences are shown in SEQ ID NO:11 and SEQ ID NO:35.

In another example, the presence or absence of at least one germline mutation in CTHRC1 can be detected using PCR. In some instances, PCR can be performed using oligonucleotide primers (e.g., having SEQ ID NOS:25-26) to detect the presence of at least one germline mutation in CTHRC1 (e.g., having a polynucleotide sequence of SEQ ID NO:17). The presence or absence of a germline mutation in CTHRC1 can be determined by comparing the polynucleotide sequence obtained from the biological sample with a polynucleotide sequence obtained from a control (i.e., a wild-type sequence). A control or wild-type CTHRC1 polynucleotide sequence is shown in SEQ ID NO:15.

In another aspect, the presence of at least one mutation in MSR1, ASCC1, or CTHRC1 can be determined using an antibody-based detection method. In some instances, a biological sample can be screened for the presence of a MSR1 mutation using an antibody (e.g., a monoclonal antibody) that specifically binds to a polypeptide having SEQ ID NO:9, SEQ ID NO:10, or SEQ ID NOS:31-34 using ELISA, for example. In other instances, a biological sample can be screened for the presence of a MSR1 mutation using an antibody that specifically binds to an MSR1 polypeptide, but not necessarily a polypeptide having SEQ ID NO:9, SEQ ID NO:10, or SEQ ID NOS:31-34. In such instances, recovered MSR1 polypeptides can be sequenced (using standard techniques) and then compared to a control or wild-type sequence (e.g., SEQ ID NOS:4-6) to screen for the presence of a mutation (e.g., a Leu254Val amino acid change or an Arg293X amino acid change).

In other instances, a biological sample can be screened for the presence of a ASCC1 mutation using an antibody (e.g., a monoclonal antibody) that specifically binds to a polypeptide having SEQ ID NO:12 or SEQ ID NO:38 using ELISA, for example. In other instances, a biological sample can be screened for the presence of a ASCC1 mutation using an antibody that specifically binds to an ASCC1 polypeptide, but not necessarily a polypeptide having SEQ ID NO:12 or SEQ ID NO:38. In such instances, recovered ASCC1 polypeptides can be sequenced (using standard techniques) and then compared to a control or wild-type sequence (e.g., SEQ ID NO:12 or SEQ ID NO:36) to screen for the presence of a mutation (e.g., an Asn290Ser amino acid change).

In other instances, a biological sample can be screened for the presence of a CTHRC1 mutation using an antibody (e.g., a monoclonal antibody) that specifically binds to a polypeptide having SEQ ID NO:18 using ELISA, for example. In other instances, a biological sample can be screened for the presence of a CTHRC1 mutation using an antibody that specifically binds to an CTHRC1 polypeptide, but not necessarily a polypeptide having SEQ ID NO:18. In such instances, recovered CTHRC1 polypeptides can be sequenced (using standard techniques) and then compared to a control or wild-type sequence (e.g., SEQ ID NO:16) to screen for the presence of a mutation (e.g., a Gln44Pro amino acid change).

In another aspect, a subject's risk for developing a precursor lesion (e.g., BE), an esophageal cancer (e.g., EAC), or both, can be determined by detecting the level of at least one protein or polypeptide associated with inflammation and/or the cell cycle, such as CCND1. This aspect of the present disclosure is based, at least in part, on the discovery that: (1) nuclear CCND1 levels were increased in MSR1-mutation carriers (as compared to controls); and (2) decreased MSR1 protein levels were associated with overexpression of nuclear CCND1 in BE tissues in MSR1-mutation carriers (but not in control normal epithelium). Although not wishing to be bound by theory, this discovery suggests a linkage of inflammation to the cell cycle and a potential etiology for BE via loss of control of the G1-S transition consistent with checkpoint-mediated cell cycle delays. Thus, in some instances, the level of nuclear CCND1 in a biological sample can be detected by standard techniques (e.g., Western blotting). The results can then be compared to a control, wherein an increased level of nuclear CCND1 in the biological sample can indicate that the subject is a MSR1-mutation carrier and, thus, at increased risk for developing a precursor lesion, an esophageal cancer, or both.

In another aspect, a determination can be made as to whether the subject is at increased risk of developing a precursor lesion (e.g., BE), an esophageal cancer (e.g., EAC), or both based on the detected presence or absence of one or more mutations in MSR1, ASCC1 and CTHRC1. In some instances, a subject that is positive for at least one germline mutation in MSR1, ASCC1, and CTHRC1 may be at increased risk for developing a precursor lesion, an esophageal cancer, or both. An increased risk for developing a precursor lesion, an esophageal cancer, or both, can mean that the subject has a risk for developing a particular precursor lesion, an esophageal cancer, or both, that is greater that the risk for developing that particular precursor lesion, an esophageal cancer, or both, in the population as a whole. In some instances, the population as a whole can include individuals sharing the same sex, age range, physical health, medical condition, or geographic location. For example, the population as a whole can refer to adult humans residing in the United States. In other instances, the presence or absence of one or more mutations in MSR1, ASCC1, and CTHRC1 in BE can be evaluated by comparison to a reference or control. The reference or control may be normal esophageal epithelium obtained from the same individual, at the same time, or at a different time. Alternatively, the reference or control may be the presence or absence of one or more mutations in a biological sample comprising cells characteristic of BE obtained from the same individual at a different time, which would permit molecular changes to be monitored over time. It is also envisioned that comparison of mutations may be made with reference to a normal range established using normal cells from a population of individuals.

In one example, the presence of at least one germline mutation in MSR1, ASCC1, and/or CTHRC1 may indicate that a subject has an increased risk of possessing a heritable form of a precursor lesion, an esophageal cancer, or both.

In another example, the presence of at least one somatic mutation in MSR1, ASCC1, and/or CTHRC1 may indicate that a subject has an increased risk of possessing a non-inherited form of a precursor lesion, an esophageal cancer, or both.

In another aspect, a method for determining a treatment strategy for a subject can comprise the step of predicting the subject's risk for developing an esophageal cancer, a precursor lesion, or both. The method can include the steps of: obtaining a biological sample from the subject; determining in the biological sample the presence of at least one germline mutation; and determining whether the subject exhibits a low risk or a high risk of developing a precursor lesion, an esophageal cancer, or both. Methods for selecting a subject, obtaining a biological sample therefrom, and detecting the presence or absence of at least one mutation (e.g., a germline or somatic mutation) in a gene associated with a precursor lesion, an esophageal cancer, or both (e.g., MSR1, ASCC1 and CTHRC1) are described above. If it is determined that the subject exhibits a low risk for developing a precursor lesion, an esophageal cancer, or both, a first therapeutic intervention may be administered to the subject. If it is determined that the subject exhibits a high risk for developing a precursor lesion, an esophageal cancer, or both, a second therapeutic intervention may be administered to the subject.

In some instances, the subject may be at low risk for developing a precursor lesion, an esophageal cancer, or both, if the presence of at least one mutation in MSR1, ASCC1 and/or CTHRC1 is not detected. In this instance, the subject may be advised of a first therapeutic intervention comprising a routine of periodic screening visits (e.g., to the subject's physician) using, for example, a standard upper endoscopy procedure. The term “low risk” as used herein can mean that the subject has a risk for developing a particular precursor lesion, an esophageal cancer, or both, that is less than or about equal to the risk for developing that particular precursor lesion, an esophageal cancer, or both, in the population as a whole (described above), or as compared to a reference or control (also described above). In one example, a reference or control can include a subject (or group of subjects) that is free of at least one germline (or somatic) mutation in MSR1 (e.g., in exon 5 and/or exon 6), ASCC1 (e.g., in exon 8) and/or CTHRC1 (e.g., in exon 1). In another example, a reference or control can include a subject (or group of subjects) having a wild-type sequence of MSR1 (e.g., SEQ ID NO:1, SEQ ID NO:2 or SEQ ID NO:3), ASCC1 (e.g., SEQ ID NO:11 or SEQ ID NO:35), and/or CTHRC1 (e.g., SEQ ID NO:15). In other instances, the first therapeutic intervention may simply include omission of any further medical or preventive action by the subject and/or the subject's physician.

In another instance, the subject may be at high risk for developing a precursor lesion, an esophageal cancer, or both, if the presence of at least one mutation in MSR1, ASCC1 and/or CTHRC1 is detected. In this instance, the subject may be advised of a second therapeutic intervention. In some instances, the second therapeutic intervention can include preventative measures, such as behavior modification (e.g., diet modification) and/or increased surveillance. It is known, for example, that gastroesophageal reflux disease is highly associated with the development of BE. Thus, in some instances, a subject determined to be at high risk of developing BE and/or EAC may be encouraged to pursue certain behavior modifications to reduce the incidence of acid reflux, such as avoiding spicy or acidic foods and sleeping on an inclined surface. Additionally or alternatively, a subject determined to be at high risk of developing BE and/or EAC may be prescribed one or more acid reflux medications, such as an H₂-receptor antagonist (e.g., Cimetidine) or a proton pump inhibitor (e.g., Omeprazole). In other instances, a patient determined to be at high risk of developing BE and/or EAC may subject to increased surveillance (e.g., of the lower esophageal tissue) via a regularly scheduled series of endoscopy procedures beginning, e.g., at an early age. For instance, the subject may need to undergo an endoscopy procedure every twelve months or less, every 2-3 years, or greater. The determination of a regular endoscopy screening protocol is within the skill of one in the art. In other instances, the second therapeutic intervention can include radiation treatment. In further instances, the second therapeutic intervention can include a chemotherapy regimen. In still further instances, the second therapeutic intervention can include surgery. It will be appreciated that several other factors may be taken into account when formulating a therapeutic intervention, such as the stage of the precursor lesion and/or an esophageal cancer, the size of the tumor, and the subject's general health.

In another aspect, a method for treating a subject with a precursor lesion, an an esophageal cancer, or both, can comprise the following steps: obtaining a biological sample from the subject; determining in the biological sample the presence of at least one mutation (e.g., a germline or somatic mutation); and administering a therapeutic intervention to the subject when the presence of one or more mutations is detected in the biological sample. In some instances, the steps of subject selection and obtaining a biological sample from the subject can be performed as described above. In other instances, detection of at least one mutation in MSR1, ASCC1, and/or CTHRC1 can also be performed as described above.

In further instances, a subject determined to be a carrier of at least one mutation (e.g., a germline mutation) in one or more of MSR1, ASCC1, and CTHRC1 can be selected to receive a therapeutic intervention. The type and duration of therapy administered to the subject can be determined by one skilled in the art. As described above, for example, a subject that is a carrier of mutation in MSR1, ASCC1, and/or CTHRC1 may be selected to receive one or combination of standard therapies, such as radiation therapy, chemotherapy, and surgery.

It will be appreciated that the methods of the present disclosure can be performed in conjunction with other assays (e.g., for EAC) including, for example, performing conventional histological analysis of a blood sample (e.g., where detection of the presence and degree of dysplasia is further indicative that the subject is at increased risk for developing EAC), determining the age of the subject (e.g., where if a human subject is more than about 60 years-old, this is further indicative that the subject is at risk for developing EAC, and where the greater the age of the patient, the higher his or her risk), and/or determining the BE segment length (e.g., where a length of 3 cm or greater is further indicative that the subject has an increased risk of developing EAC).

### Kits

Another aspect of the present disclosure can include a kit for predicting a subject's risk for developing a precursor lesion (e.g., BE), an esophageal cancer (e.g., EAC), or both. One skilled in the art will recognize components of kits suitable for carrying out a method (or methods) of the present disclosure. The agents in the kit can encompass reagents (e.g., primers and probes for detection of mutations in MSR1, ASCC1 and CTHRC1) for carrying out a method (or methods) of the present disclosure. In some instances, a kit of the present disclosure can include a probe for detecting the presence of at least one germline mutation in MSR1, ASCC1 and/or CTHRC1. For example, the kit can include a primer pair (e.g., SEQ ID NO: 19 and SEQ ID NO: 20) selected to amplify a germline mutation in exon 5 of MSR1; or a primer pair (e.g., SEQ ID NO:21 and SEQ ID NO:22) selected to amplify a germline mutation in exon 6 of MSR1. In another example, a kit can include a primer pair (e.g., SEQ ID NO:23 and SEQ ID NO:24) selected to amplify a germline mutation in exon 8 of ASCC1. In yet another example, a kit can include a primer pair (e.g., SEQ ID NO:25 and SEQ ID NO:26) selected to amplify a germline mutation in exon 1 of CTHRC1. Kits may also include additional agents suitable for detecting, measuring and/or quantitating the amount of PCR amplification, for example.

Optionally, a kit of the present disclosure can include instructions for performing the method(s). Optional elements of a kit of the present disclosure can include suitable buffers, control reagents, containers, or packaging materials. The reagents of the kit may be in containers in which the reagents are stable, e.g., in lyophilized form or stabilized liquids. The reagents may also be in single use form, e.g., for the performance of an assay for a single subject.

Kits of the present disclosure may comprise one or more computer programs that may be used in practicing the methods of the present disclosure. For example, a computer program may be provided that takes the output from microplate reader or realtime-PCR gels or readouts and prepares a calibration curve from the optical density observed in the wells, capillaries or gels, and compares these densitometric or other quantitative readings to the optical density or other quantitative readings in wells, capillaries, or gels with test samples.

In addition to the clinical uses discussed herein, kits of the present disclosure can be used for experimental applications.

The following example is for the purpose of illustration only and is not intended to limit the scope of the claims, which are appended hereto.

### Example

### Methods

Our study (2005-2010), approved by respective institutions' review board for research participants, involved prospective recruitment of all 298 consenting adults with histologically proven BE, EAC, or both, as well as families with 2 or more cases with BE, EAC, or both from 16 academic and community hospitals and clinics nationally (two-thirds originated from Cleveland Clinic, Cleveland, Ohio, and Johns Hopkins Medical Institutions, Baltimore, Md.; <1% of research participants declined participation). All BE cases were long segment. For discordant sibling pair studies, the non-affected sibling had endoscopy documented unaffected status. Only white participants of northern or western European descent were selected and sex-matched in cases and controls.

Identification of Loci Using Genome-Wide Mapping Methods

Model-Free Linkage Analysis

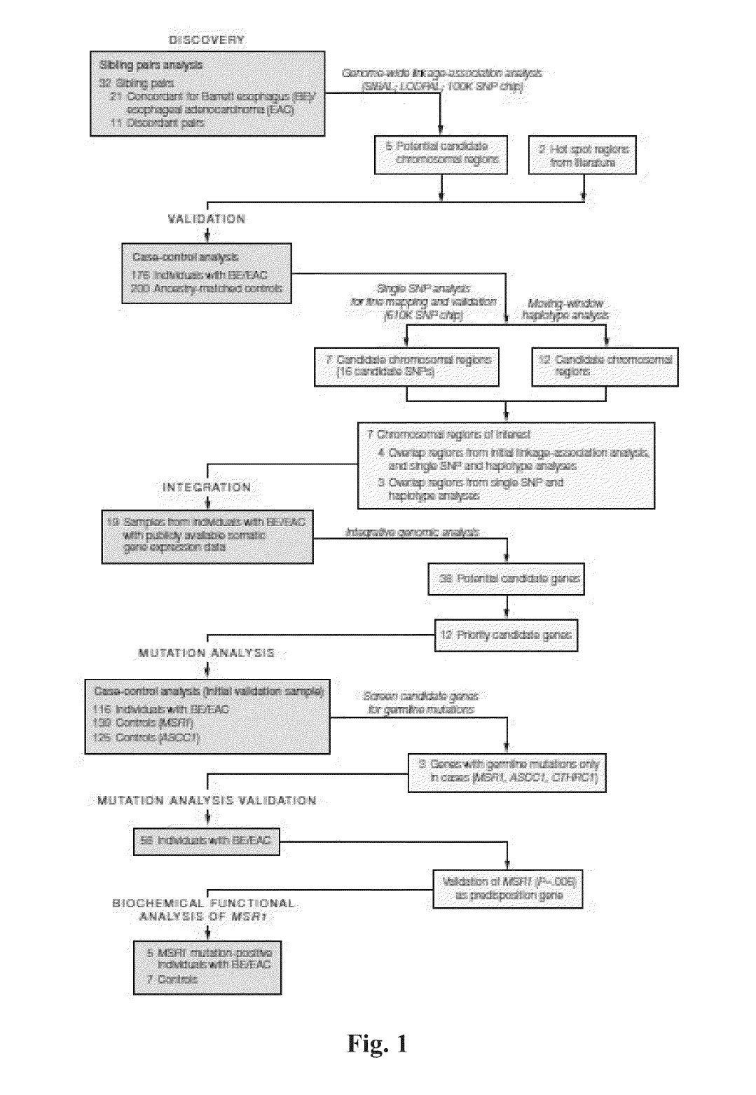

Twenty-one concordant-affected sibling pairs (42 individuals with BE/EAC) and 11 discordant sibling pairs (11 with BE/EAC and 11 without BE/EAC) (2005-2006) were genotyped using Affymetrix GeneChip Human Mapping 100K SNP set (Affymetrix, Santa Clara, Calif.) (FIG. 1). Significant linkage to chromosomal regions found by 1 model-free linkage analysis method was self-replicated by a second model-free linkage analysis approach, which is used for small sample-sized data sets. Genomic regions were considered potentially interesting when −log₁₀ P value(pP)≧2.2 by SIBPAL analysis 13 had logarithm of odds >3.2 by LODPAL analysis. These regions from this pilot linkage-association analysis were considered “potentially interesting” and served as regions to be validated (FIG. 1).

Independent Validation and Fine Mapping Significant Regions

It is standard in this field to single out significant genomic regions from pilot analysis to follow up with increased sample sizes from independent cases (validation), more genetic markers (fine mapping), or both in a second validation stage (FIG. 1). We followed this strategy of independent validation and fine mapping of the “potentially interesting” regions identified by the pilot linkage-association analysis. We also paid particular attention to 2 additional regions (1q23 and 8p22), because these regions were found previously to be frequently somatically lost (by array comparative genomic hybridization) in EAC or gastroesophageal junction cancers.

Population substructure of cases and controls was determined by PLINK and EIGENSTRAT. Analysis using EIGENSTRAT software and principal component analysis identified the top eigenvalues from the 376 available eigenvalues. Regression analyses were used to allow for potential population substructure by 2 separate analyses (PLINK-derived and EIGENSTRAT-derived analyses). After population substructure was assessed to be similar (>85%), SNP association analyses of the above targeted genomic regions were performed with an independent validation series totaling 176 patients with BE/EAC and 200 ancestry-matched population controls (2007-2010) whose SNP data were derived from the denser Illumina Human610-Quad BeadChips (Illumina Inc, Hayward, Calif.). Although we only were validating specific regions, we reasoned that it would be more cost-efficient to genotype all markers in a commercially available Chip instead of creating a new automation process for a reduced marker set. If the underlying genetic effect had been negligible, we would not have expected to see any savings on the average sample size, but fortunately the underlying genetic effect was large enough to warrant savings on sample size.

Statistical simulations have indicated that haplotype analysis with multiple SNPs may be more powerful than single SNP analysis because multiple alleles at different loci on the same chromosome that are in linkage disequilibrium (LD) are likely to interact with each other to result in a phenotype. Thus, haplotype analysis was performed using PLINK26 to predict the most likely haplotypes and those that were significantly associated with the BE/EAC phenotype. To account for type I error, an empirical P value corrected for testing multiple markers was obtained by permuting (10,000 permutations) the affectation status across the individual genotypes, as described in PLINK.

Integrating Information from Significant Regions with Publicly Available Somatic Gene Expression Data Sets

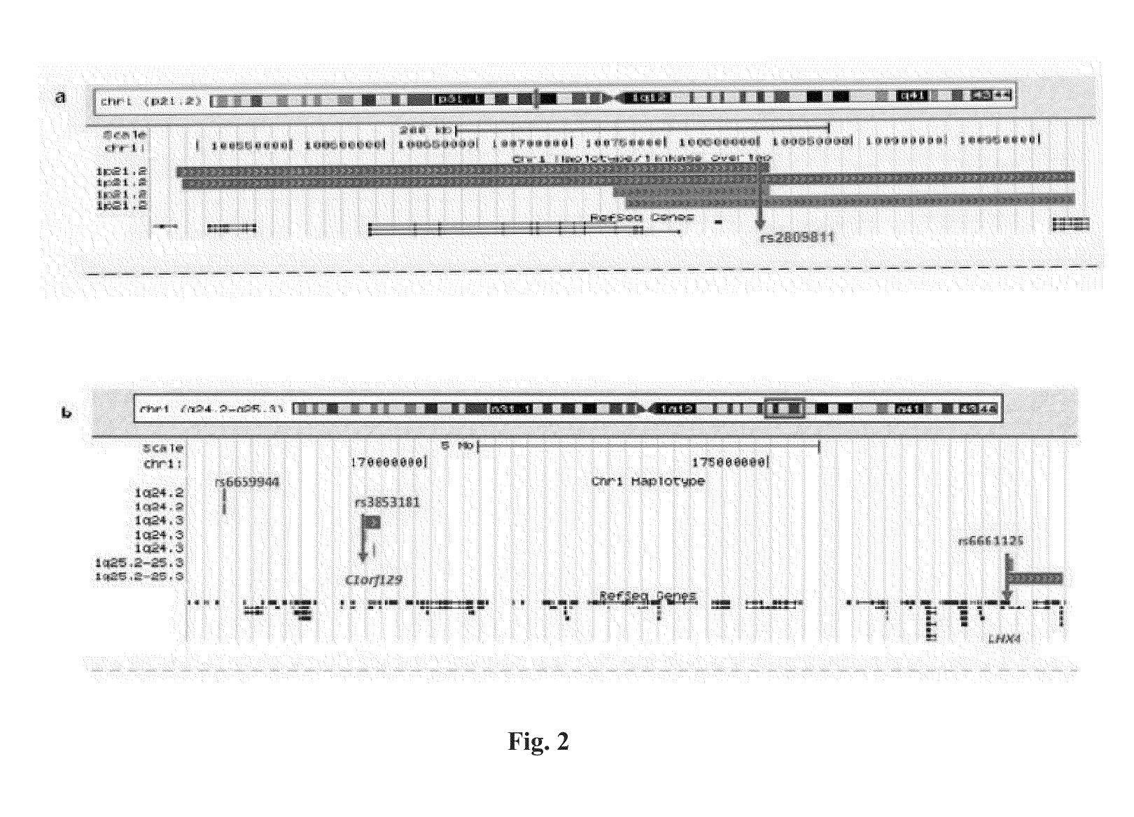

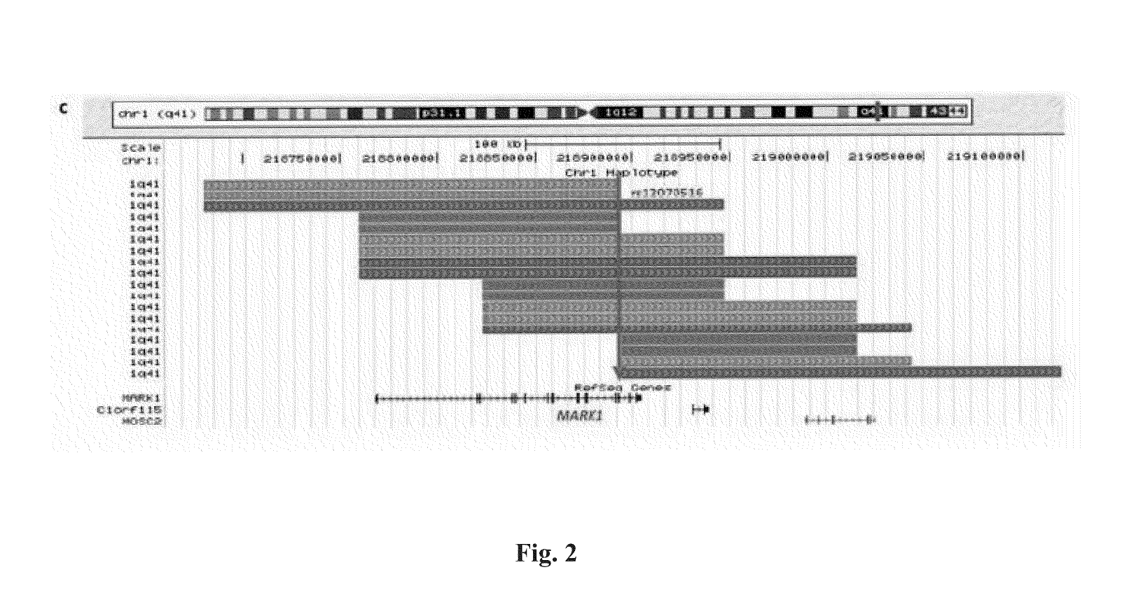

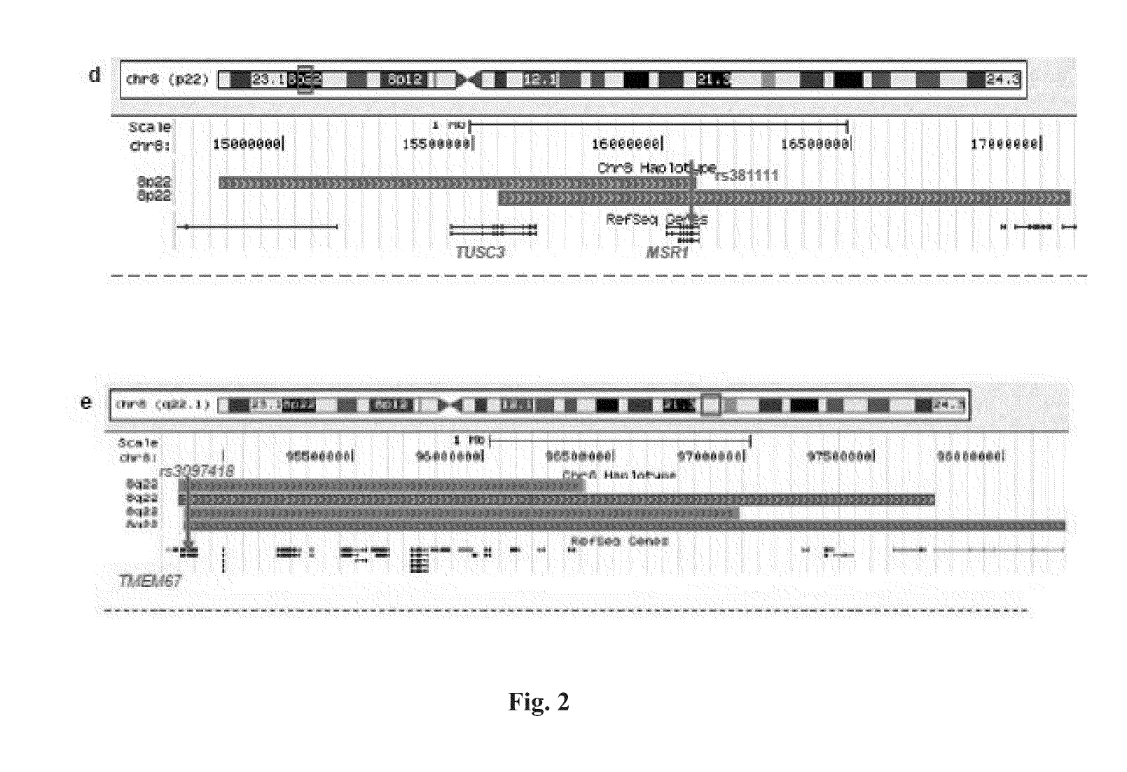

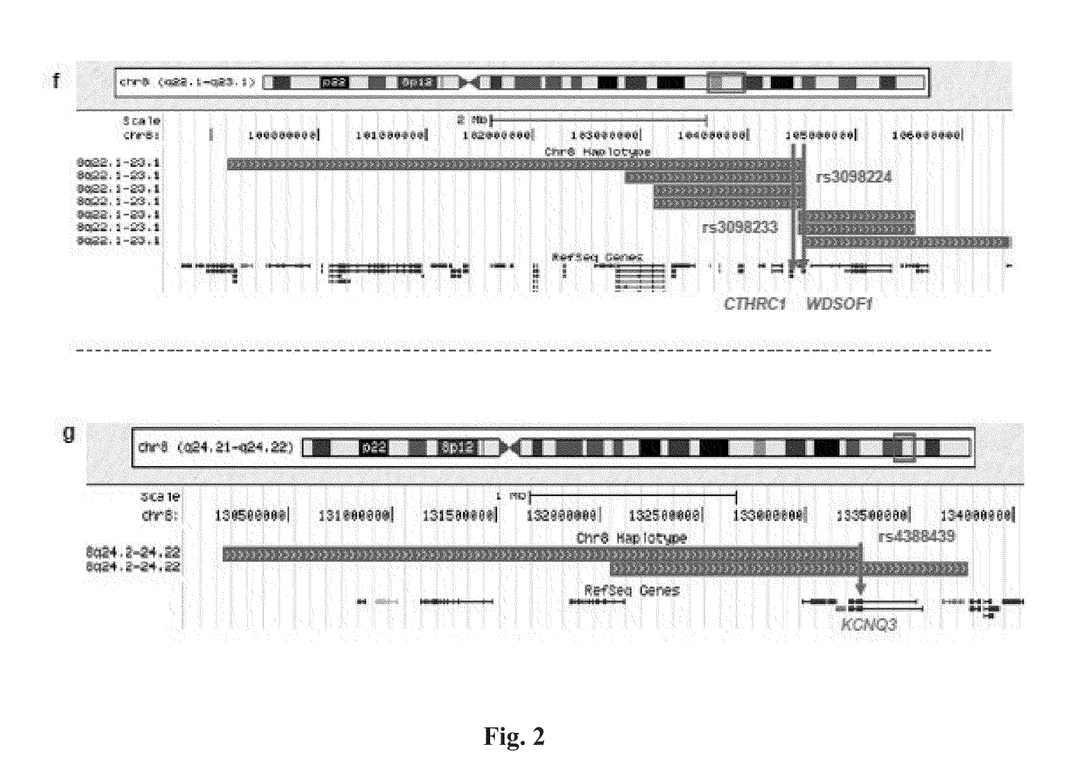

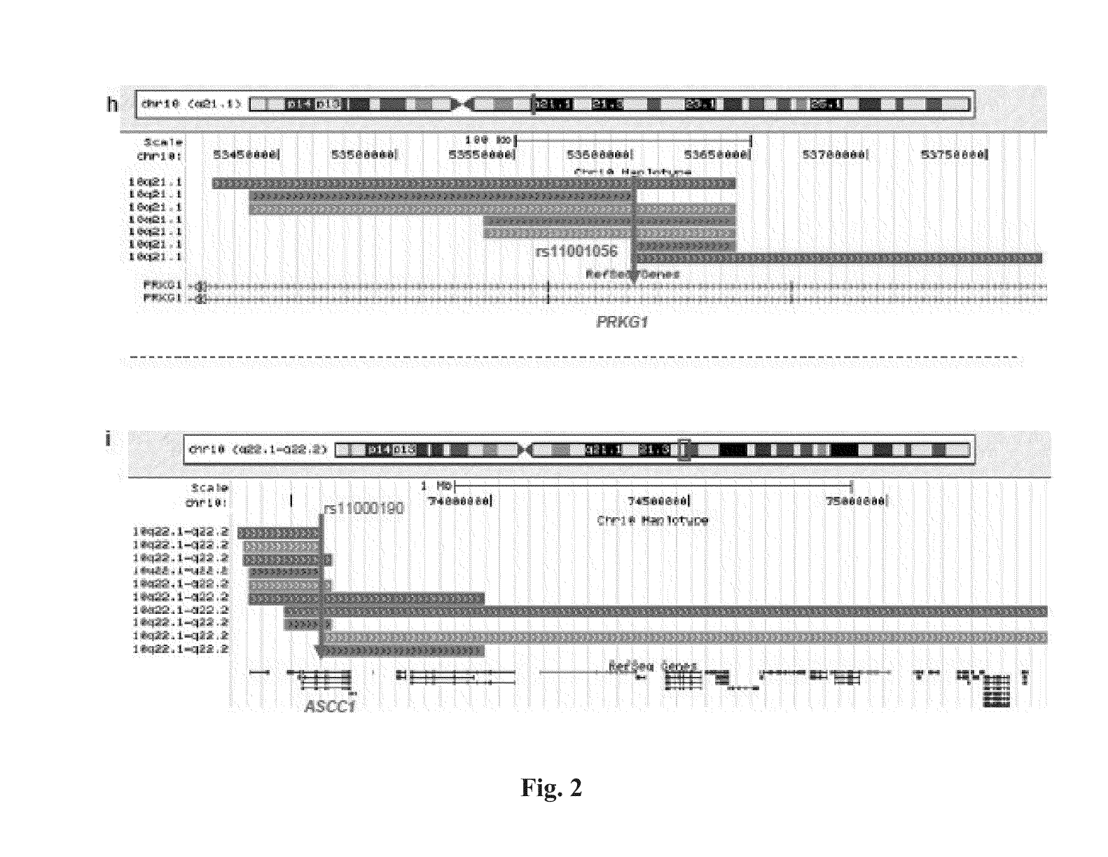

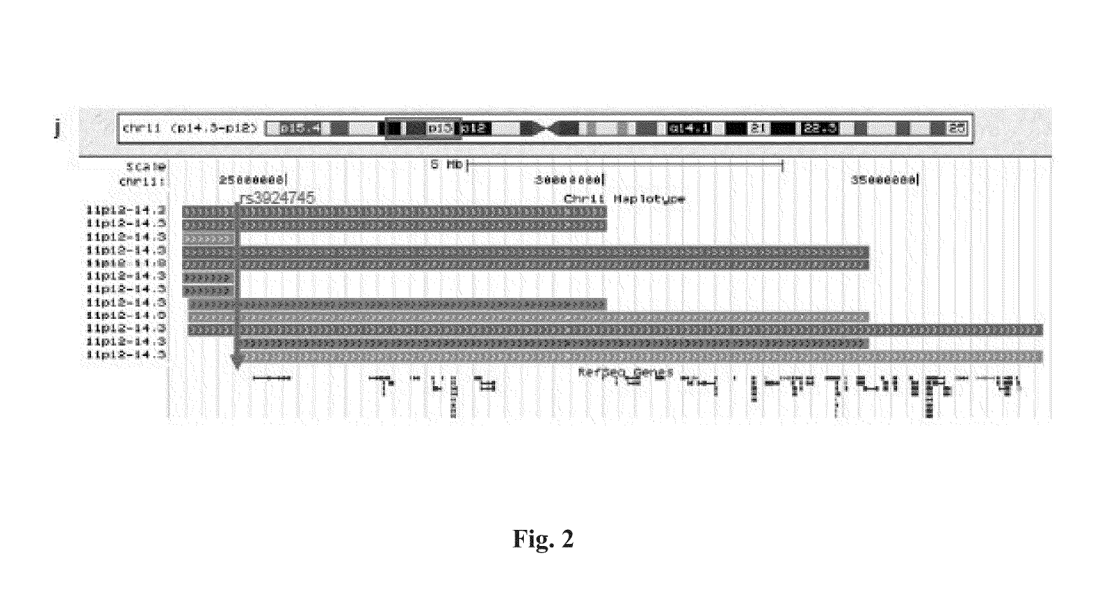

To narrow in on one or a subset of genes within and in proximity to the significant SNPs/haplotypes germane to BE/EAC (by tissue-specific expression in oncologic pathways and for functional-genomic validation), we integrated our significant regions with publicly available somatic gene expression data derived from 19 patients with BE/EAC (GDS3472 or GSE13083), followed up by unsupervised hierarchical clustering of genes within 250 kb flanking the significant SNPs and haplotypes across BE/EAC and unaffected individuals (FIGS. 2A-J).

TABLE 1
Significant Single SNP Association Results From Pilot-Combined Linkage-Association
and Independent Validation Case-Control Analyses in Patients With BE/EAC.

| | | | | Association | Linkage | Single SNP | Gene at the |
|---|---|---|---|---|---|---|---|
| | | Location, | | Analysis, | Analysis, LOD | Association, | Significant |
| Region | db SNP | Build 36.1 | P Value | FDR-Corrected | Scores (pP^{sp})^{a} | pP^{Asscnb} | SNP |
| 1q21.2 | rs2809811 | 100,805,788 | <.001 | 0.0185 | | 4.80 | |
| 1q24.1-25.3 | rs10494465 | 164,810,325 | .007 | | 4.31 (2.17) | | |
| | rs950302 | 165,350,678 | .004 | | 4.38 (2.40) | | |
| | rs10489191 | 165,772,769 | .004 | | 3.68 (2.36) | | |
| | rs10489211 | 166,579,946 | .004 | | 3.32 (2.35) | | |
| | rs6659944 | 167,046,343 | <.001 | 0.0175 | | 5.03 | |
| | rs10494476 | 167,267,368 | .009 | | 3.10 (2.06) | | |
| | rs3853181 | 169,241,785 | <.001 | 0.0198 | | 4.47 | C1orf129 |
| | rs6661125 | 178,493,273 | <.001 | 0.0175 | | 5.06 | LHX4 |
| 1q41 | rs10209401 | 217,912,676 | <.001 | 0.0214 | | 4.07 | DIRC3 |
| | rs12070516 | 218,894,580 | <.001 | 0.0199 | | 4.75 | MARK1 |
| | rs12062054 | 229,020,356 | <.001 | 0.0199 | | 4.93 | |
| | rs2355230 | 237,851,538 | .007 | | 4.12 (2.18) | | |
| | rs4498839 | 243,586,955 | <.001 | 0.0173 | | 5.22 | KIF268 |
| 8p22 | rs381111 | 16,090,070 | <.001 | 0.0253 | | 3.29 | MSR1 |
| 8q21.11-22 | rs4469448 | 75,457,415 | .01 | | 3.19 (2.01) | | |
| | rs4739755 | 81,665,497 | .01 | | 3.39 (2.00) | | |
| | rs3097418 | 94,851,657 | <.001 | 0.0253 | | 3.33 | TMEM67 |
| 8q22.1-24.22 | rs3098224 | 104,515,622 | <.001 | 0.0253 | | 3.24 | WDSOF1 |
| | rs3098233 | 104,463,670 | <.001 | 0.0253 | | 3.24 | CTHRC1 |
| | rs4388439 | 133,277,554 | <.001 | 0.0253 | | 3.10 | KCNQ3 |
| 10q21-22 | rs11001056 | 53,599,547 | <.001 | 0.0463 | | 3.04 | PRKG1 |
| | rs2050381 | 55,029,991 | <.001 | | 3.604 (3.52) | | |
| | rs10509021 | 56,518,547 | <.001 | | 2.566 (4.00) | | |
| | rs11000190 | 73,577,964 | <.001 | 0.0262 | | 3.46 | ASCC1 |
| 11q21 | rs7107185 | 94,342,447 | <.001 | | 4.588 (3.40) | | |
| | rs1255537 | 94,958,558 | <.001 | | 4.54 (3.52) | | |
| 11q25 | rs11223500 | 132,917,451 | <.001 | 0.0500 | | 3.23 | OPCML |

Abbreviations. BE, Barrett esophagus: EAC, esophageal adenocarcinoma: FDR, false discovery rate: LOD, logarithm of odds: SNP, single-nucleotide polymorphism.
^{a}LOD score was derived from LODPAL [symbol] p [symbol] indicates −log₁₀(P value) derived from SIBPAL (considered −log₁₀(P value) ≧2.00). from analysis of the 32 sibling pairs (21 concordant-affected sibling pairs and 11 discordant sibling pairs). [symbol]
^{b}pP [symbol] indicatess −log₁₀(P value) derived from the validation case-control association analysis using independent n = 376 (comprising 176 cases and 200 controls) [symbol]
indicates data missing or illegible when filed

TABLE 2
Haplotypes Significantly Associated With BE/EAC vs. Controls.

| Chromosome Region | Haplotype | SNPs | P Value | Significant Genes |
|---|---|---|---|---|
| 1q21.2 | 12212 | rs3806237, rs12060945, rs2270694, rs12722868, rs2809811^{b} | <.001 | |
| | 22121 | rs12060945, rs2270694, rs12722868, rs2809811^{b}, rs34552536 | <.001 | |
| | 221 | rs2270694, rs12722868, rs2809811^{b} | <.001 | |
| | 212 | rs12722868, rs2809811^{b}, rs34552536 | <.001 | |
| 1q24.2 | 122 | rs6659944^{b}, rs12067866, rs12069349 | <.001 | |
| | 222 | rs6659944^{b}, rs12067866, rs12069349 | <.001 | |
| 1q24.3 | 222 | rs16828284, rs3853181^{b}, rs1800822 | <.001 | C1orf129 |
| | 21212 | rs16828284, rs3853181^{b}, rs1800822, rs2066530, rs2066536 | <.001 | C1orf129 |
| | 222 | rs3853181^{b}, rs1800822, rs2066530 | <.001 | C1orf129 |
| 1q25.2-25.3 | 22112 | rs6661125^{b}, rs17300107, rs6670868, rs16856123, rs17302632 | <.001 | LHX4 |
| | 122 | rs6661125^{b}, rs17300107, rs6670868 | <.001 | LHX4 |
| 1q41 | 11112 | rs1338775, rs6694126, rs17007991, rs12070516^{b}, rs17008285 | <.001 | MARK1 |
| | 11122 | rs6694126, rs17007991, rs12070516^{b}, rs17008285, rs17008643 | <.001 | MARK1 |
| | 11222 | rs17007991, rs12070516^{b}, rs17008285, rs17008643, rs17008806 | <.001 | MARK1 |
| | 12222 | rs12070516^{b}, rs17008285, rs17008643, rs17008806, rs3806325 | <.001 | MARK1 |
| | 22222 | rs6694126, rs17007991, rs12070516^{b}, rs17008285, rs17008643 | <.001 | MARK1 |
| | 1122 | rs17007991, rs12070516^{b}, rs17008285, rs17008643 | <.001 | MARK1 |
| | 2222 | rs17007991, rs12070516^{b}, rs17008285, rs17008643 | <.001 | MARK1 |
| | 1112 | rs6694126, rs17007991, rs12070516^{b}, rs17008285 | <.001 | MARK1 |
| | 1222 | rs12070516^{b}, rs17008285, rs17008643, rs17003806 | <.001 | MARK1 |
| | 2222 | rs6694126, rs17007991, rs12070516^{b}, rs17008285 | <.001 | MARK1 |
| | 2222 | rs1338775, rs6694126, rs17007991, rs12070516^{b} | <.001 | MARK1 |
| | 1111 | rs1338775, rs6694126, rs17007991, rs12070516^{b} | <.001 | MARK1 |
| | 222 | rs6694126, rs17007991, rs12070516^{b} | <.001 | MARK1 |
| | 112 | rs17007991, rs12070516^{b}, rs17008285 | <.001 | MARK1 |
| | 222 | rs17007991, rs12070516^{b}, rs17008285 | <.001 | MARK1 |
| | 122 | rs12070516^{b}, rs17008285, rs17008643 | <.001 | MARK1 |
| | 222 | rs12070516^{b}, rs17003285, rs17008643 | <.001 | MARK1 |
| | 111 | rs12070516^{b}, rs17008285, rs17008643, rs17008806, rs3806325 | <.001 | MARK1 |
| 8p22 | 22221 | rs4265186, rs268387, rs354521, rs354517, rs381111^{b} | .002 | MSR1 |
| | 22112 | rs354521, rs354517, rs381111^{b}, rs2959634, rs2959631 | .004 | MSR1 |
| 8q22.1 | 21222 | rs3097422, rs3097418^{b}, rs6989157, rs6987276, rs4392869 | .002 | TMEM67 |
| | 12222 | rs3097418^{b}, rs6989157, rs6987276, rs4392869, rs987036 | .002 | TMEM67 |
| | 212 | rs3097422, rs3097418^{b}, rs6989157 | <.001 | TMEM67 |
| | 221 | rs3097418^{b}, rs6989157, rs6987276 | <.001 | TMEM67 |
| 8q22.1-23.1 | 22122 | rs3098233^{b}, rs3098224^{b}, rs3098218, rs3098212, rs2959025 | <.001 | CTHRC1, WDSOF1 |
| | 11211 | rs3098233^{b}, rs3098224^{b}, rs3098218, rs3098212, rs2959025 | <.001 | CTHRC1, WDSOF1 |
| | 22112 | rs6988793, rs6987078, rs3098233^{b}, rs3098224^{b}, rs3098218 | .001 | CTHRC1, WDSOF1 |
| | 12212 | rs6987078, rs3098233^{b}, rs3098224^{b}, rs3098218, rs3098212 | .002 | CTHRC1, WDSOF1 |
| | 21121 | rs6987078, rs3098233^{b}, rs3098224^{b}, rs3098218, rs3098212 | .002 | CTHRC1, WDSOF1 |
| | 22211 | rs2959644, rs6988793, rs6987078, rs3098233^{b}, rs3098224^{b} | .002 | CTHRC1, WDSOF1 |
| | 12112 | rs3098224^{b}, rs3098218, rs3098212, rs2959025, rs2057452 | .002 | WDSOF1 |
| 8q24.2-24.22 | 22222 | rs6989059, rs6986982, rs6988942, rs6989209, rs4388439^{b} | <.001 | KCNQ3 |
| | 12212 | rs6986982, rs6988942, rs6989209, rs4388439^{b}, rs3843561 | .002 | KCNQ3 |
| 10q21.1 | 12212 | rs11000400, rs11000436, rs11000798, rs11001056^{b}, rs11001210 | .003 | PRKG1 |
| | 22222 | rs11001056^{b}, rs11001210, rs11001213, rs11001447, rs11001702 | .002 | PRKG1 |
| | 2212 | rs11000436, rs11000798, rs11001056^{b}, rs11001210 | .002 | PRKG1 |
| | 2122 | rs11000798, rs11001056^{b}, rs11001210, rs11001213 | .003 | PRKG1 |
| | 221 | rs11000436, rs11000798, rs11001056^{b} | <.001 | PRKG1 |
| | 212 | rs11000798, rs11001056^{b}, rs11001210 | <.001 | PRKG1 |
| | 222 | rs11001056^{b}, rs11001210, rs11001213 | <.001 | PRKG1 |

Prioritized Candidate Gene Analysis

A final list of biologically plausible candidate genes (“priority” candidate genes) was then scanned for germline mutations in BE/EAC cases and compared with ancestry-matched population controls (FIG. 1). Genes with mutations in cases but not in controls were screened in an independent validation series of 58 cases prospectively accrued from outpatient endoscopy units (2010) (FIG. 1).

TABLE 3
Primers Used To Amplify MSR1, ASCC1 and CTHRC1 genes.

| | | | | Direct Seq. | LS |
|---|---|---|---|---|---|
| | | | SEQ | Annealing | Annealing |
| Gene | Exon | Direction | ID NO | Temperature | Temperature |
| MSR1 | 5 | F | SEQ ID | 56.5 | |
| | | | NO: 19 | | |
| MSR1 | 5 | R | SEQ ID | 56.5 | |
| | | | NO: 20 | | |
| MSR1 | 6 | F | SEQ ID | | 61 |
| | | | NO: 21 | | |
| MSR1 | 6 | R | SEQ ID | | 61 |
| | | | NO: 22 | | |
| ASCC1 | 8 | F | SEQ ID | | 60 |
| | | | NO: 23 | | |
| ASCC1 | 8 | R | SEQ ID | | 60 |
| | | | NO: 24 | | |
| CTHRC1 | 1 | F | SEQ ID | 63.5 | |
| | | | NO: 25 | | |
| CTHRC1 | 1 | R | SEQ ID | 63.5 | |
| | | | NO: 26 | | |

Seq: Sequencing
LS: LightScanner

MSR1 and CCND1 Protein Levels and Cell Lines

Proteins were extracted from immortalized lymphoblastoid cells obtained from patients with BE/EAC and normal controls. After processing, protein lysates were loaded onto sodium dodecyl sulfate-polyacrylamide gel electrophoresis gels. Antibodies specific to CCND1 (Cell Signaling Technology Inc, Danvers, Mass.), MSR1 (Abcam, Cambridge, Mass.), and α-tubulin (Sigma-Aldrich, St Louis, Mo.) were used for Western blotting.

Wild-type MSR1 or pCMV-FLAG empty vector were transiently transfected into MSR1-null HEK293 cells using Lipofectamine LTX and Plus Reagent (Invitrogen, Carlsbad, Calif.). Cells were harvested after 24 hours and lysates (30 μg of protein) were analyzed by Western blotting using antibodies against FLAG (Sigma-Aldrich, 1:1000), CCND1 (Thermo-Fisher Scientific Inc, Waltham, Mass., 1:200), and α-tubulin (Sigma-Aldrich, 1:5000).

CCND1 Immunohistochemical Analysis

CCND1 immunohistochemical analysis was performed using an avidin-biotin complex immunoperoxidase technique.

### Results

Linkage and Association Analyses

A pilot combined linkage-association analysis based on modification of established criteria revealed 5 candidate regions (1q24.1-25.3, 1q41, 8q21.11-22, 10q21-22, and 11q21) (Table 1 and FIG. 1).

Subsequently, we performed a validation study in an independent series of 176 cases and 200 controls (FIG. 1), using a denser SNP-marker set but focusing only on the germline regions of interest and the 2 somatically lost hot spot regions (1q23 and 8p22). We were able to validate 4 (1q24.1-25.3, 1q41, 8q21.11-22, and 10q21-22) of these 5 pilot-derived germline candidate regions, while excluding one (11q21). Three additional loci at locations remote from the pilot linkage peaks (1q21.2, 8p22, and 11q25) were also found (Table 1). The most significant SNPs from this validative association analysis were located within or in the vicinity of the most promising pilot-derived linkage peaks (Table 1).

Moving-Window Haplotype Analysis

Haplotype and LD analysis conducted on the 176 cases and 200 controls confirmed our findings—any single SNP that was significant in the above single SNP analysis always revealed a haplotype block containing significant SNPs within the haplotypes (at least in LD) (P<0.005) (Table 2). In the haplotype analysis, we considered regions of highest priority as those haplotypes exhibiting significance across multiple SNPs. The combined P values from the significant single SNPs and the significant haplotypes facilitated prioritization of regions of interest for further follow-up. There were 4 significant regions that overlapped from the linkage, single SNP association, and haplotype-LD analyses (1q24.1-25.3 [encompassing 1q24.2, 1q24.3, and 1q25.2-25.3 fine-mapped regions], 1q41, 8q21.11-22 [encompassing 8q21.11-22 and 8q22.1-24.22 fine-mapped regions], and 10q21-22). Additionally, there were 3 significant regions that overlapped in the single SNP association and haplotype analyses (1q21.2, 8p22, and 10q22.1). Thus, we selected these regions, shown in Table 2, as “regions of interest” (FIG. 1, Table 4, and Table 5). Each of these regions contained SNPs that were statistically significant at P<0.005 and also had multiple haplotype windows showing significance (P<0.01).

TABLE 4
Analysis Of Single SNP Case-Control and Family-Based Tests and Genes
in Specific Loci that are Associated with FBE/EAC (p < 0.05).

| | | | | Linkage | Association | Gene at the |
|---|---|---|---|---|---|---|
| | | Location | | Analysis, LOD | (single SNP), | significant |
| Region | db SNP | (Build 36.1) | (pvalue) | scores (pP) | pP^{Asscn} | SNP |
| 1q21.2 | rs2809811 | 100,805,788 | 1.60E−05 | | 4.8 | |
| 1q24.1-25.2 | rs986362 | 163,283,373 | 0.026 | 3.42 (1.59) | | |
| | rs952375 | 163,825,599 | 0.014 | 3.78 (1.86) | | |
| | rs10494465 | 164,810,325 | 0.007 | 4.31 (2.17) | | |
| | rs950302 | 165,350,678 | 0.004 | 4.38 (2.40) | | |
| | rs10489191 | 165,772,769 | 0.004 | 3.68 (2.36) | | |
| | rs10489211 | 166,579,946 | 0.004 | 3.32 (2.35) | | |
| | rs6659944 | 167,046,343 | 9.41E−06 | | 5.03 | |
| | rs10494476 | 167,267,368 | 0.009 | 3.10 (2.06) | | |
| | rs10489181 | 167,979,471 | 0.012 | 3.04 (1.93) | | |
| | rs2184085 | 168,667,119 | 0.014 | 3.26 (1.84) | | |
| | rs10489235 | 169,099,216 | 0.016 | 4.35 (1.80) | | |
| | rs3853181 | 169,241,785 | 3.42E−05 | | 4.47 | C1orf129 |
| | rs6661125 | 178,493,273 | 8.70E−06 | | 5.06 | LJHX4 |
| 1q41 | rs12070516 | 218,894,580 | 1.78E−05 | | 4.75 | MARK1 |
| | rs12062054 | 229,020,356 | 1.18E−05 | | 4.93 | |
| | rs959175 | 234,819,755 | 0.0257 | 3.46 (1.59) | | |
| | rs946933 | 235,215,696 | 0.0221 | 3.52 (1.66) | | |
| | rs2998400 | 235,667,678 | 0.0173 | 3.65 (1.76) | | |
| | rs2275691 | 236,048,643 | 0.013 | 3.49 (1.89) | | |
| | rs1564505 | 236,482,853 | 0.0153 | 3.18 (1.81) | | |
| | rs1910296 | 236,893,424 | 0.0135 | 3.21 (1.87) | | |
| | rs10495442 | 237,321,760 | 0.0113 | 3.37 (1.95) | | |
| | rs2355230 | 237,851,538 | 0.0066 | 4.12 (2.18) | | |
| | rs4498839 | 243,586,955 | 6.05E−06 | | 5.22 | KIF26B |
| | rs10209401 | 217,912,676 | 8.54E−05 | | 4.07 | DIRC3 |
| 8p22 | rs381111 | 16,090,070 | 0.00051 | | 3.29 | MSR1 |
| 8q21.11-22 | rs1434930 | 69,712,502 | 0.017 | 2.08 (1.77) | | |
| | rs10504448 | 70,551,629 | 0.028 | 2.04 (1.55) | | |
| | rs1873547 | 70,887,215 | 0.019 | 2.47 (1.73) | | |
| | rs10504472 | 71,402,782 | 0.019 | 2.29 (1.71) | | |
| | rs10504501 | 72,232,207 | 0.023 | 2.00 (1.64) | | |
| | rs10504516 | 72,927,679 | 0.03 | 1.82 (1.53) | | |
| | rs7837478 | 73,323,448 | 0.044 | 1.37 (1.35) | | |
| | rs349337 | 73,606,967 | 0.022 | 2.21 (1.65) | | |
| | rs7837090 | 74,383,710 | 0.017 | 2.79 (1.78) | | |
| | rs4469448 | 75,457,415 | 0.01 | 3.19 (2.01) | | |
| | rs1526658 | 77,430,751 | 0.011 | 3.27 (1.96) | | |
| | rs720302 | 81,015,879 | 0.01 | 3.34 (1.99) | | |
| | rs4739755 | 81,665,497 | 0.01 | 3.39 (1.99) | | |
| | rs3097418 | 94,851,657 | 0.00047 | | 3.33 | TMEM67 |
| 8q24 | rs3098224 | 104,515,622 | 0.00058 | | 3.24 | WDSOF1 |
| | rs3098233 | 104,463,670 | 0.00058 | | 3.24 | CTHRC1 |
| | rs3098212 | 104,533,275 | 0.00315 | | 2.5 | near a large del |
| | rs4388439 | 133,277,554 | 0.00079 | | 3.1 | KCNQ3 |
| 10q21-22 | rs11001056 | 53,599,547 | 0.00092 | | 3.04 | PRKG1 |
| | rs930507 | 54,198,022 | 0.005 | 2.22 (2.33) | | |
| | rs2050381 | 55,029,991 | 0 | 3.604 (3.52) | | |
| | rs10509021 | 56,518,547 | 0 | 2.566 (4.00) | | |
| | rs10509047 | 57,725,580 | 0 | 1.522 (3.70) | | |
| | rs10509092 | 60,189,566 | 0.023 | 0.793 (1.65) | | |
| | rs11000190 | 73,577,964 | 0.00035 | | 3.46 | ASCC1 |
| 11q21 | rs7107185 | 94,342,447 | 0 | 4.59 (3.40) | | |
| | rs1255537 | 94,958,558 | 0 | 4.54 (3.52) | | |
| 11q25 | rs11223500 | 132,917,451 | 0.00059 | | 3.23 | closest gene OPCML |

LOD score: Derived from LODPAL
pP; −log₁₀(Pvalue) derived from SIBPAL
pP^{Asscn}: −log₁₀(Pvalue) derived from Case-control association analysis

TABLE 5
Haplotypes Significantly Associated With FBE/EAC (p < 0.05).

| Chr -region | Window | Haplotype | SNPs | Pvalue | Genes (significant) |
|---|---|---|---|---|---|
| 1q21.2 | 5 | 12212 | rs3806237 - rs12060945 - rs2270694 - rs12722868 - rs2809811 | 4.99E−05 | |
| | 5 | 22121 | rs12060945 - rs2270694 - rs12722868 - rs2809811 - rs34552536 | 4.42E−05 | |
| | 3 | 221 | rs2270694 - rs12722868 - rs2809811 | 4.65E−05 | |
| | 3 | 212 | rs12722868 - rs2809811 - rs34552536 | 5.56E−05 | |
| 1q24.2 | 3 | 122 | rs6659944 - rs12067866 - rs12069349 | 1.68E−05 | |
| | 3 | 222 | rs6659944 - rs12067866 - rs12069349 | 1.68E−05 | |
| 1q24.3 | 3 | 222 | rs16828284 - rs3853181 - rs1800822 | 2.66E−05 | C1orf129 |
| | 5 | 21212 | rs16828284 - rs3853181 - rs1800822 - rs2066530 - rs2066536 | 7.46E−05 | C1orf129 |
| | 3 | 222 | rs3853181 - rs1800822 - rs2066530 | 5.55E−05 | C1orf129 |
| 1q25.2-25.3 | 5 | 22112 | rs6661125 - rs17300107 - rs6670868 - rs16856123 - rs17302632 | 2.38E−05 | LHX4 |
| | 3 | 122 | rs6661125 - rs17300107 - rs6670868 | 2.57E−05 | LHX4 |
| 1q41 | 5 | 11112 | rs1338775 - rs6694126 - rs17007991 - rs12070516 - rs17008285 | 0.000469 | MARK1 |
| | 5 | 11122 | rs6694126 - rs17007991 - rs12070516 - rs17008285 - rs17008643 | 0.000223 | MARK1 |
| | 5 | 11222 | rs17007991 - rs12070516 - rs17008285 - rs17008643 - rs17008806 | 0.000313 | MARK1 |
| | 5 | 12222 | rs12070516 - rs17008285 - rs17008643 - rs17008806 - rs3806325 | 0.000234 | MARK1 |
| | 5 | 22222 | rs6694126 - rs17007991 - rs12070516 - rs17008285 - rs17008643 | 0.000176 | MARK1 |
| | 4 | 1122 | rs17007991 - rs12070516 - rs17008285 - rs17008643 | 0.000137 | MARK1 |
| | 4 | 2222 | rs17007991 - rs12070516 - rs17008285 - rs17008643 | 0.00014 | MARK1 |
| | 4 | 1112 | rs6694126 - rs17007991 - rs12070516 - rs17008285 | 0.000184 | MARK1 |
| | 4 | 1222 | rs12070516 - rs17008285 - rs17008643 - rs17008806 | 0.000205 | MARK1 |
| | 4 | 2222 | rs6694126 - rs17007991 - rs12070516 - rs17008285 | 0.000207 | MARK1 |
| | 4 | 2222 | rs1338775 - rs6694126 - rs17007991 - rs12070516 | 0.000534 | MARK1 |
| | 4 | 1111 | rs1338775 - rs6694126 - rs17007991 - rs12070516 | 0.00092 | MARK1 |
| | 4 | 2222 | rs6694126 - rs17007991 - rs12070516 | 0.000811 | |
| | 3 | 222 | rs17007991 - rs12070516 - rs17008285 | 4.99E−05 | MARK1 |
| | 3 | 112 | rs1338775 - rs6694126 - rs17007991 - rs12070516 - rs17008285 | 0.000114 | MARK1 |
| | 3 | 222 | rs6694126 - rs17007991 - rs12070516 - rs17008285 - rs17008643 | 0.000132 | MARK1 |
| | 3 | 122 | rs17007991 - rs12070516 - rs17008285 - rs17008643 - rs17008806 | 0.000145 | MARK1 |
| | 3 | 222 | rs12070516 - rs17008285 - rs17008643 - rs17008806 - rs3806325 | 0.000159 | MARK1 |
| | 3 | 111 | rs6694126 - rs17007991 - rs12070516 - rs17008285 - rs17008643 | 0.00036 | MARK1 |
| | 3 | 222 | rs17007991 - rs12070516 - rs17008285 - rs17008643 | 0.000279 | |
| 8p22 | 5 | 22221 | rs4265186 - rs268387 - rs354521 - rs354517 - rs381111 | 0.001819 | MSR1 |
| | 5 | 22112 | rs354521 - rs354517 - rs381111 - rs2959634 - rs2959631 | 0.004069 | MSR1 |
| | 5 | 12211 | rs268387 - rs354521 - rs354517 - rs381111 - rs2959634 | 0.01951 | MSR1 |
| | 5 | 22222 | rs4265186 - rs268387 - rs354521 - rs354517 - rs381111 | 0.03523 | MSR1 |
| | 5 | 11212 | rs381111 - rs2959634 - rs2959631 - rs4388484 - rs439374 | 0.04275 | MSR1 |
| | 5 | 21221 | rs268387 - rs354521 - rs354517 - rs381111 - rs2959634 | 0.04683 | MSR1 |
| 8q22.1 | 5 | 21222 | rs3097422 - rs3097418 - rs6989157 - rs6987276 - rs4392869 | 0.001573 | TMEM67 |
| | 5 | 12222 | rs3097418 - rs6989157 - rs6987276 - rs4392869 - rs987036 | 0.001794 | TMEM67 |
| | 5 | 22112 | rs3097418 - rs6989157 - rs6987276 - rs4392869 - rs987036 | 0.009254 | TMEM67 |
| | 5 | 12212 | rs3097422 - rs3097418 - rs6989157 - rs6987276 - rs4392869 | 0.01782 | TMEM67 |
| | 5 | 22211 | rs3097422 - rs3097418 - rs6989157 - rs6987276 - rs4392869 | 0.01819 | TMEM67 |
| | 5 | 12212 | rs2957792 - rs2681297 - rs3097422 - rs3097418 - rs6989157 | 0.021 | TMEM67 |
| | 5 | 21221 | rs2681297 - rs3097422 - rs3097418 - rs6989157 - rs6987276 | 0.02128 | TMEM67 |
| | 5 | 12122 | rs2681297 - rs3097422 - rs3097418 - rs6989157 - rs6987276 | 0.02615 | TMEM67 |
| | 5 | 22122 | rs3097418 - rs6989157 - rs6987276 - rs4392869 - rs987036 | 0.0299 | TMEM67 |
| | 5 | 21221 | rs4961199 - rs2957792 - rs2681297 - rs3097422 - rs3097418 | 0.03577 | TMEM67 |
| | 5 | 21121 | rs4961199 - rs2957792 - rs2681297 - rs3097422 - rs3097418 | 0.04001 | TMEM67 |
| | 3 | 212 | rs2681297 - rs3097422 - rs3097418 | 0.02614 | TMEM67 |
| | 3 | 212 | rs3097422 - rs3097418 - rs6989157 | 0.000896 | TMEM67 |
| | 3 | 221 | rs3097418 - rs6989157 - rs6987276 | 0.000354 | TMEM67 |
| | 3 | 121 | rs2681297 - rs3097422 - rs3097418 | 0.0194 | TMEM67 |
| | 3 | 221 | rs2681297 - rs3097422 - rs3097418 | 0.02112 | TMEM67 |
| 8q22.1-23.1 | 5 | 22122 | rs3098233 - rs3098224 - rs3098218 - rs3098212 - rs2959025 | 0.000641 | CTHRC1, WDSOF1 |
| | 5 | 11211 | rs3098233 - rs3098224 - rs3098218 - rs3098212 - rs2959025 | 0.000865 | CTHRC1, WDSOF1 |
| | 5 | 22112 | rs6988793 - rs6987078 - rs3098233 - rs3098224 - rs3098218 | 0.001397 | CTHRC1, WDSOF1 |
| | 5 | 12212 | rs6987078 - rs3098233 - rs3098224 - rs3098218 - rs3098212 | 0.001606 | CTHRC1, WDSOF1 |
| | 5 | 21121 | rs6987078 - rs3098233 - rs3098224 - rs3098218 - rs3098212 | 0.002473 | CTHRC1, WDSOF1 |
| | 5 | 22211 | rs2959644 - rs6988793 - rs6987078 - rs3098233 - rs3098224 | 0.002525 | CTHRC1, WDSOF1 |
| | 5 | 22221 | rs2959646 - rs2959644 - rs6988793 - rs6987078 - rs3098233 | 0.005795 | CTHRC1 |
| | 5 | 11221 | rs6988793 - rs6987078 - rs3098233 - rs3098224 - rs3098218 | 0.01965 | CTHRC1 |
| | 5 | 22212 | rs2959646 - rs2959644 - rs6988793 - rs6987078 - rs3098233 | 0.01998 | CTHRC1 |
| | 5 | 21212 | rs2959646 - rs2959644 - rs6988793 - rs6987078 - rs3098233 | 0.02773 | CTHRC1 |
| | 5 | 22122 | rs2959644 - rs6988793 - rs6987078 - rs3098233 - rs3098224 | 0.03125 | WDSOF1 |
| | 5 | 21221 | rs6988793 - rs6987078 - rs3098233 - rs3098224 - rs3098218 | 0.03265 | WDSOF1 |
| | 5 | 21112 | rs2959646 - rs2959644 - rs6988793 - rs6987078 - rs3098233 | 0.03353 | CTHRC1 |
| | 5 | 11122 | rs2959644 - rs6988793 - rs6987078 - rs3098233 - rs3098224 | 0.04399 | WDSOF1 |
| | 5 | 12112 | rs3098224 - rs3098218 - rs3098212 - rs2959025 - rs2957452 | 0.002345 | WDSOF1 |
| | 5 | 21222 | rs3098224 - rs3098218 - rs3098212 - rs2959025 - rs2957452 | 0.009235 | WDSOF1 |
| 8q24.2-24.22 | 5 | 22222 | rs6989059 - rs6986982 - rs6988942 - rs6989209 - rs4388439 | 0.000357 | KCNQ3 |
| | 5 | 12212 | rs6986982 - rs6988942 - rs6989209 - rs4388439 - rs3843561 | 0.001806 | KCNQ3 |
| | 5 | 22122 | rs6988942 - rs6989209 - rs4388439 - rs3843561 - rs6988193 | 0.01526 | KCNQ3 |
| | 5 | 21221 | rs6989059 - rs6986982 - rs6988942 - rs6989209 - rs4388439 | 0.01934 | KCNQ3 |
| | 5 | 22221 | rs6986982 - rs6988942 - rs6989209 - rs4388439 - rs3843561 | 0.02112 | KCNQ3 |
| | 5 | 22212 | rs6988942 - rs6989209 - rs4388439 - rs3843561 - rs6988193 | 0.04249 | KCNQ3 |
| 10q21.1 | 5 | 22122 | rs11000178 - rs11000400 - rs11000436 - rs11000798 - rs11001056 | 0.009492 | PRKG1 |
| | 5 | 12212 | rs11000400 - rs11000436 - rs11000798 - rs11001056 - rs11001210 | 0.00336 | PRKG1 |
| | 5 | 22222 | rs110010056 - rs11001210 - rs11001213 - rs11001447 - rs11001702 | 0.002523 | PRKG1 |
| | 4 | 2212 | rs11000436 - rs11000798 - rs11001056 - rs11001210 | 0.001834 | PRKG1 |
| | 4 | 2122 | rs11000798 - rs11001056 - rs11001210 - rs11001213 | 0.003178 | PRKG1 |
| | 4 | 1221 | rs11000400 - rs11000436 - rs11000798 - rs11001056 | 0.006259 | PRKG1 |
| | 3 | 221 | rs11000436 - rs11000798 - rs11001056 | 0.000315 | PRKG1 |
| | 3 | 212 | rs11000798 - rs11001056 - rs11001210 | 0.000395 | PRKG1 |
| | 3 | 222 | rs11001056 - rs11001210 - rs11001213 | 0.000217 | PRKG1 |
| 10q22.1 | 5 | 22221 | rs11000101 - rs11000108 - rs11000122 - rs11000152 - rs11000190 | 0.002789 | ASCC1 |
| | 5 | 22211 | rs11000108 - rs11000122 - rs11000152 - rs11000190 - rs11000202 | 0.002194 | ASCC1 |
| | 5 | 12222 | rs11000122 - rs11000152 - rs11000190 - rs11000202 - rs11000348 | 0.009332 | ASCC1 |
| | 5 | 21122 | rs11000152 - rs11000190 - rs11000202 - rs11000348 - rs11000828 | 0.000508 | ASCC1 |
| | 5 | 11222 | rs11000190 - rs11000202 - rs11000348 - rs11000828 - rs11000857 | 0.005365 | ASCC1 |
| | 4 | 1122 | rs11000190 - rs11000202 - rs11000348 - rs11000828 | 0.000793 | ASCC1 |
| | 4 | 2211 | rs11000122 - rs11000152 - rs11000190 - rs11000202 | 0.002387 | ASCC1 |
| | 4 | 2221 | rs11000108 - rs11000122 - rs11000152 - rs11000190 | 0.002478 | ASCC1 |
| | 3 | 221 | rs11000122 - rs11000152 - rs11000190 | 0.002791 | ASCC1 |
| | 3 | 211 | rs11000152 - rs11000190 - rs11000202 | 0.001277 | ASCC1 |
| | 3 | 112 | rs11000190 - rs11000202 - rs11000348 | 0.003436 | ASCC1 |
| 11q14 | 5 | 22112 | rs1381720 - rs12146457 - rs3924745 - rs665153 - rs2926467 | 0.00015 | |
| | 5 | 11221 | rs1381722 - rs1381720 - rs12146457 - rs3924745 - rs665153 | 0.00068 | |
| | 5 | 12212 | rs1381720 - rs12146457 - rs3924745 - rs665153 - rs2926467 | 0.001123 | |
| | 5 | 21122 | rs12146457 - rs3924745 - rs665153 - rs2926467 - rs1871684 | 0.001283 | |
| | 5 | 12211 | rs1381722 - rs1381720 - rs12146457 - rs3924745 - rs665153 | 0.002892 | |
| | 5 | 22221 | rs1871953 - rs1381722 - rs1381720 - rs12146457 - rs3924745 | 0.01282 | |
| | 5 | 22211 | rs1381722 - rs1381720 - rs12146457 - rs3924745 - rs665153 | 0.01424 | |
| | 5 | 11222 | rs3924745 - rs665153 - rs2926467 - rs1871684 - rs10837317 | 0.01727 | |
| | 5 | 21122 | rs1871953 - rs1381722 - rs1381720 - rs12146457 - rs3924745 | 0.02345 | |
| | 5 | 12222 | rs1871953 - rs1381722 - rs1381720 - rs12146457 - rs3924745 | 0.03584 | |
| | 5 | 11122 | rs1871953 - rs1381722 - rs1381720 - rs12146457 - rs3924745 | 0.03597 | |
| | 5 | 21221 | rs1871953 - rs1381722 - rs1381720 - rs12146457 - rs3924745 | 0.03889 | |
| | 5 | 11221 | rs3924745 - rs665153 - rs2926467 - rs1871684 - rs10837317 | 0.04398 | |
| | 4 | 2112 | rs12146457 - rs3924745 - rs665153 - rs2926467 | 0.000443 | |
| | 4 | 1122 | rs3924745 - rs665153 - rs2926467 - rs1871684 | 0.001273 | |
| | 4 | 1122 | rs1381722 - rs1381720 - rs12146457 - rs3924745 | 0.002176 | |
| | 4 | 2221 | rs1381722 - rs1381720 - rs12146457 - rs3924745 | 0.007036 | |
| | 4 | 1221 | rs1381722 - rs1381720 - rs12146457 - rs3924745 | 0.02511 | |
| | 3 | 221 | rs1381720 - rs12146457 - rs3924745 | 0.000223 | |
| | 3 | 211 | rs12146457 - rs3924745 - rs665153 | 0.000573 | |
| | 3 | 112 | rs3924745 - rs665153 - rs2926467 | 0.000831 | |
| | 3 | 122 | rs1381720 - rs12146457 - rs3924745 | 0.002437 | |

Within the “haplotype” column, 1 represents the major allele while 2 represents the minor allele at each respective SNP. Emboldened rs numbers: SNPs that were significant in the single SNP analysis that are also part of a significant haplotype block.

Functional-Genomic Validation

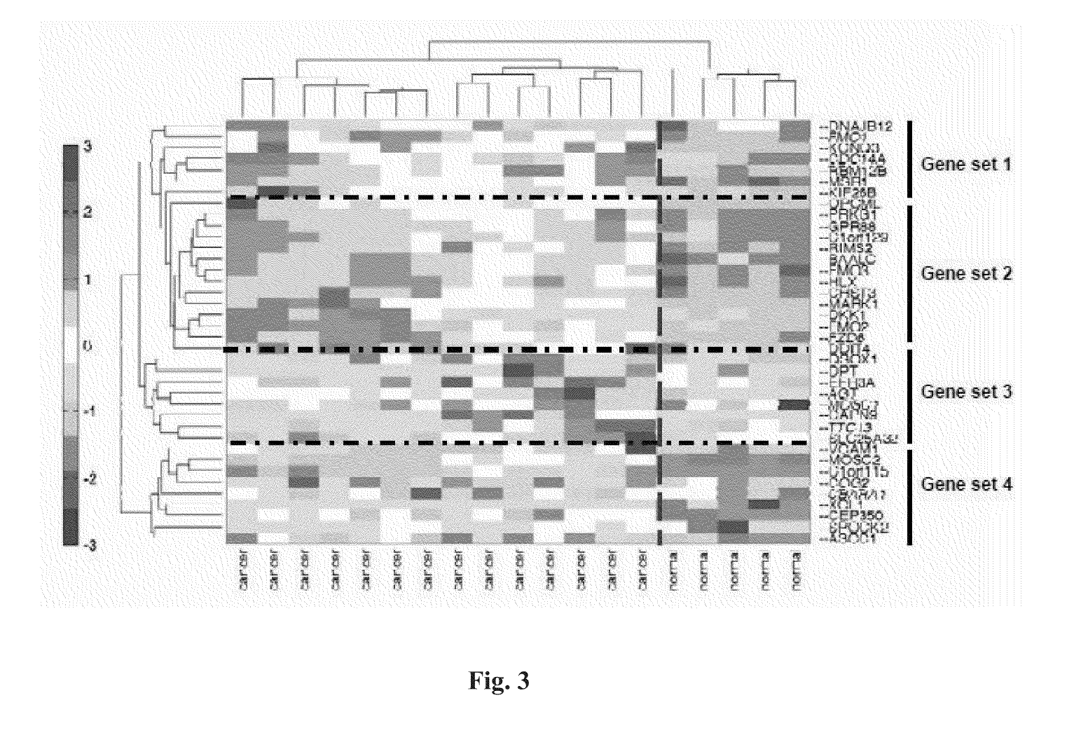

Integration of our significant SNP and haplotypes with publicly available somatic BE/EAC transcriptome data (FIG. 1) yielded 38 genes located within 250 kb flanking each significant SNP, within significant haplotypes, or both that accurately clusteredBE/EAC cases from controls (FIG. 3). An additional filtering step based on known organ-specific functions resulted in a final short list of 12 priority candidate genes (LHX4, DIRC3, MARK1, KIF26B, MSR1, TMEM67, WDSOF1, CTHRC1, KCNQ3, PRKG1, ASCC1 and OPCML), which were also functionally plausible, within our regions of interest (Table 1 and Table 2).

Mutational Analyses of Priority Candidate Genes

Mutational analyses of these 12 priority candidate genes in BE/EAC cases and controls revealed germline mutations in 3 genes (MSR1 [MIM153622], ASCC1 [NC_{—}000010.10], or CTHRC1 [MIM610635]) in 13 of 116 patients (11.2%) with BE/EAC (Table 6 and Table 3).

TABLE 6
Germline Mutations in 3 Candidate Genes in BE/EAC Cases.

| | | | 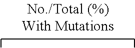 | | | |
|---|---|---|---|---|---|---|
| | | | | | Proportion of Cases | P |
| Gene | Variant | Total No. | Cases | Controls | With Variant (95% CI) | Value |
| MSR1 (mutation analysis)^{a} | c.877C > T.p.R293X | 255 | 8/116 (6.9) | 0/139 | 0.069 (0.030-0.130) | <.001 |
| MSR1 (validation)^{b} | c.877C > T.p.R293X | 197 | 2/58 (3.4) | 0/139 | 0.034 (0.004-0.120) | .09 |
| MSR1 (pooled)^{c} | c.877C > T.p.R293X | 323 | 10/184 (5.4) | 0/139 | 0.054 (0.026-0.098) | .006 |
| MSR1 (mutation analysis)^{a} | c.760C > G.p.L254V | 255 | 2/116 (1.7) | 0/139 | 0.017 (0.021-0.061) | .19 |
| ASCC1 (mutation analysis)^{a} | c.869A > G.p.N290S | 220 | 2/95 (2.1) | 0/125 | 0.021 (0.003-0.074) | .18 |
| CTHRC1 (mutation analysis)^{a} | c.131A > C.p.Q44P | 214 | 1/89 (1.1) | 0/125 | 0.011 (0.0303-0.061) | .42 |
| CTHRC1 (validation)^{b} | c.131A > C.p.Q44P | 183 | 1/58 (1.7) | 0/125 | 0.017 (0.0004-0.092) | .32 |
| CTHRC1 (pooled)^{c} | c.131A > C.p.Q44P | 272 | 2/147 (1.4) | 0/125 | 0.014 (0.0009-0.026) | .50 |

Abbreviations: ASCC1. activating signal contegrator 1 complex subunit 1: BE. Barrett esophagus: CI. confidence interval: CTHRC1. collagan triple-helo repeat-containing 1: EAC: esophageal adenocarcinoma: MSR1. macrophage scavenger receptor 1
^{a}Candidate gene mutation analysis in BE/EAC cases and controls.
^{b}MSR1 and CTHRC1 mutations validated in small independent series of BE/EAC cases.
^{c}Pooled series comprising series of cases and controls used for candidate gene mutation analysis and independent validation series.

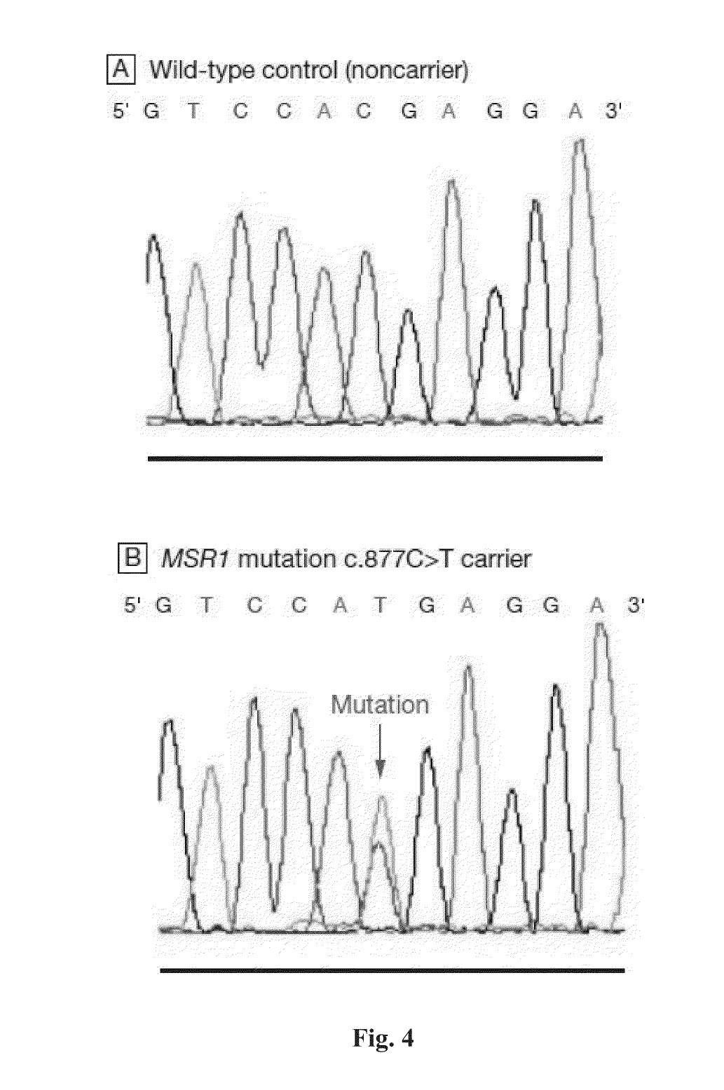

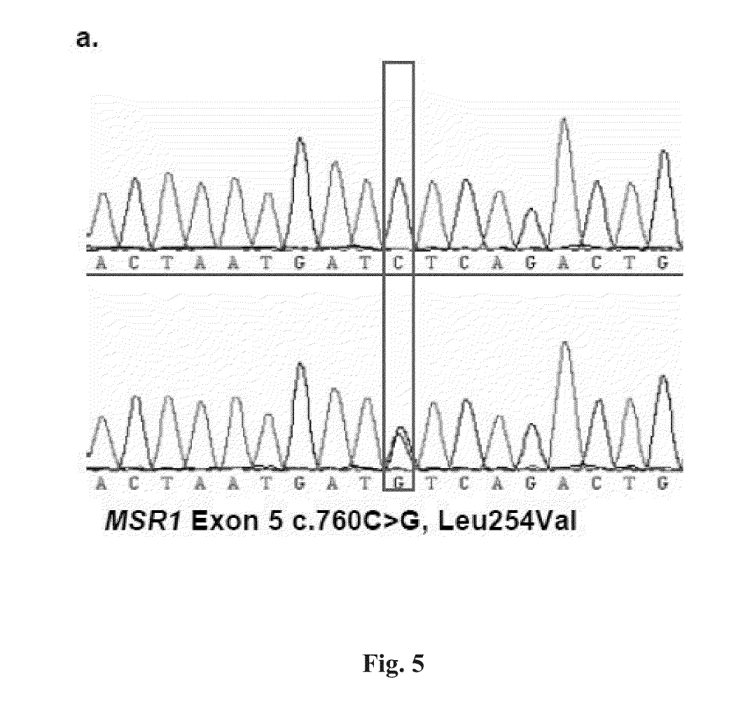

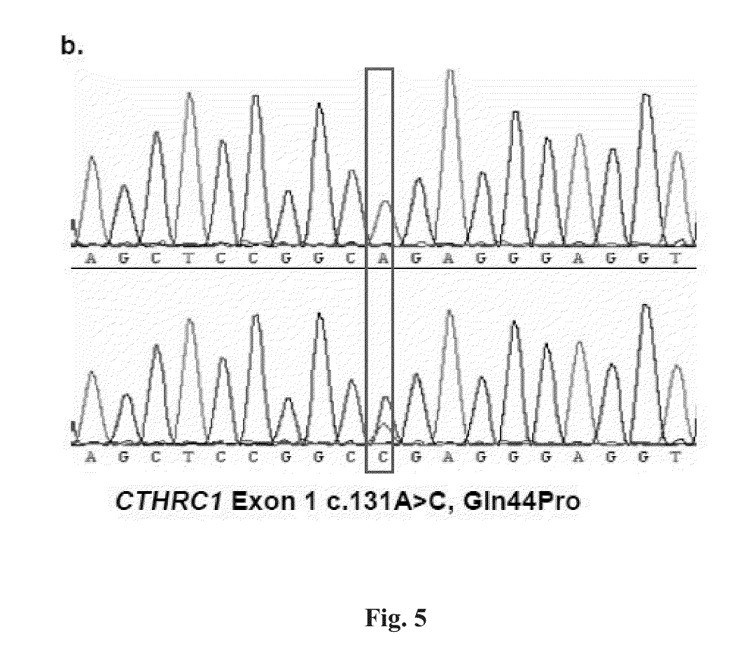

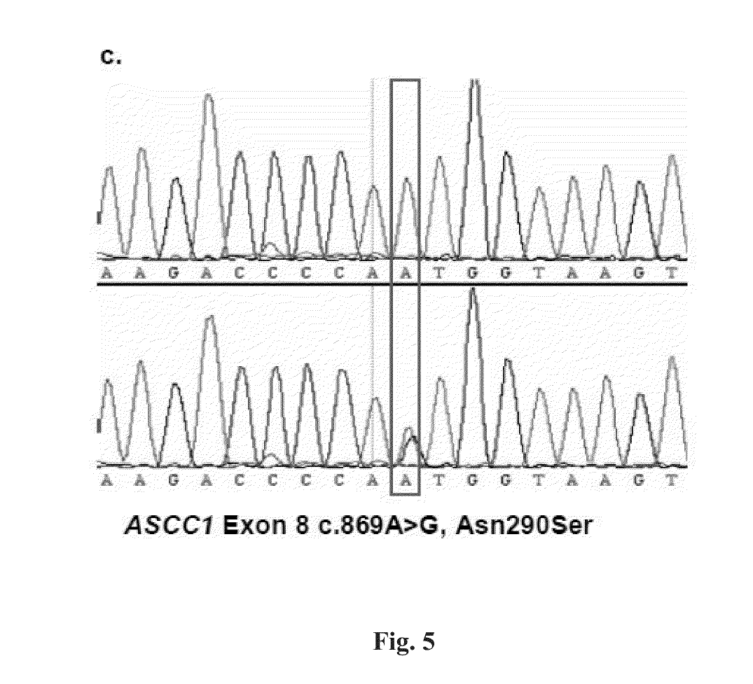

No sequence variants were found in the remaining 9 genes that were not also present to the same degree in controls. Among the 116 patients with BE/EAC, 8 patients (proportion, 0.069; 95% confidence interval [CI], 0.030-0.130; P<0.001) had a germline truncating mutation in MSR1 c.877C>T, resulting in p.R293X (FIGS. 4A-B and Table 6), and 2 additional patients (proportion, 0.017; 95% CI, 0.021-0.061; P=0.19) with BE/EAC carried germline MSR1 p.L254V (c.760C>G) in exon 5 (Table 6 and FIG. 5A). These mutations were not found in 139 ancestry-matched population controls. Additionally, we identified 2 germline missense mutations—c.869A>G in exon 8 of ASCC1, resulting in p.N290S in 2 patients (proportion, 0.021; 95% CI, 0.003-0.074; P=0.18); and c.131A>C in exon 1 of CTHRC1, resulting in p.Q44P in 1 patient (proportion, 0.011; 95% CI, 0.0003-0.061; P=0.42), neither of which were found among 125 controls (Table 6 and FIGS. 5B-C).

Independent Validation of Germline MSR1, ASCC1, and CTHRC1 Mutations

To confirm the mutations found in the 3 candidate genes (Table 6), mutational analyses were then performed in an independent series of 58 cases obtained from outpatient endoscopy units. These samples confirmed the presence of germline MSR1 c.877C>T, p.R293X mutation in 2 of 58 cases (proportion, 0.034; 95% CI, 0.004-0.120; P=0.09) and CTHRC1 c.131A>C, p.Q44P mutation in 1 of 58 cases (1.7%). After pooling the original 116 cases with the validation series of 58, a total of 10 cases with BE/EAC carried p.R293X (proportion, 0.054; 95% CI, 0.026-0.098; P=0.006) (Table 6).

MSR1 and CCND1 Protein Levels

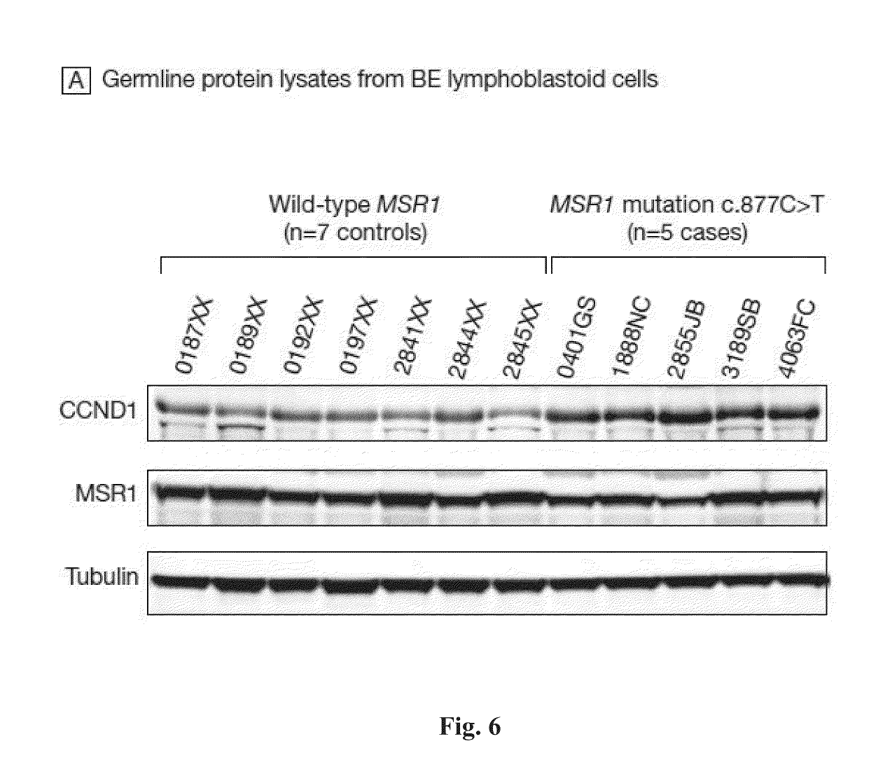

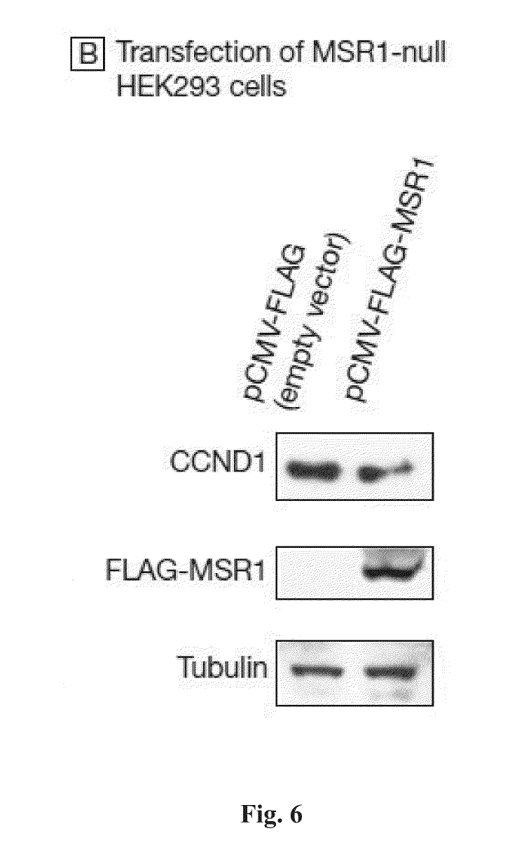

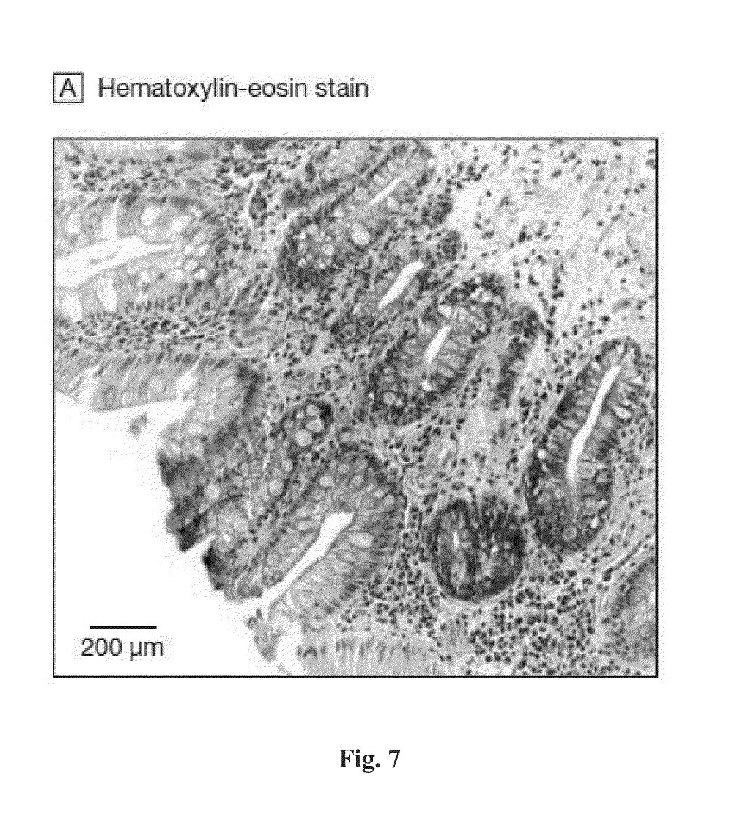

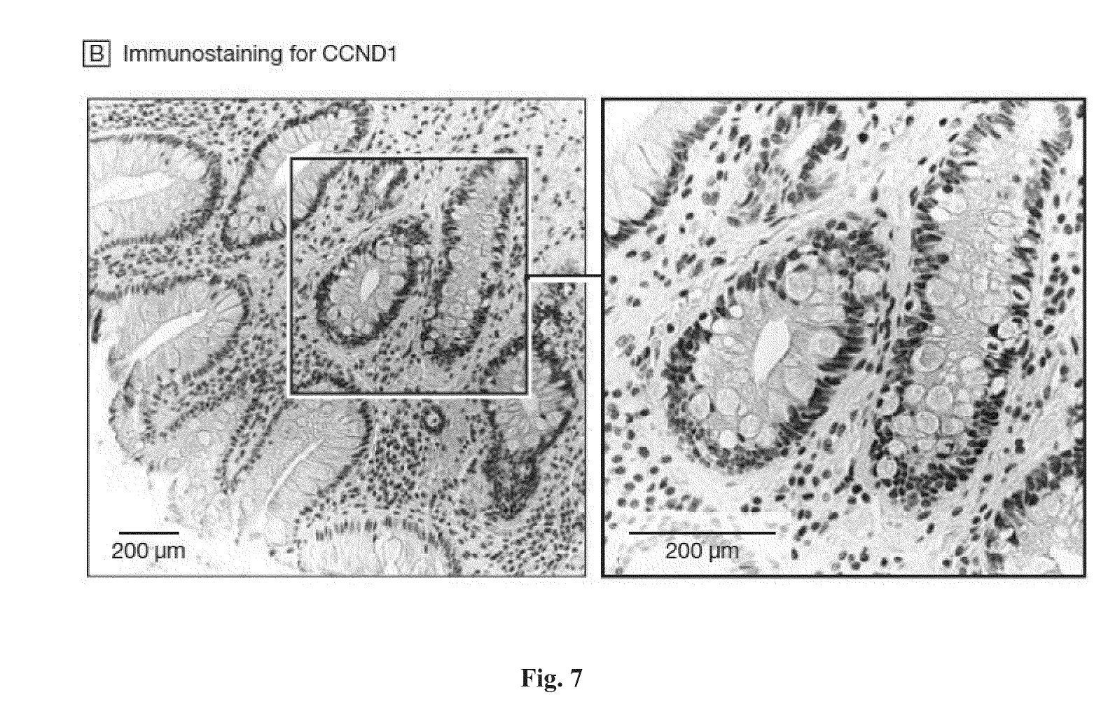

Western blotting of germline protein lysates from 5 MSR1 mutation-positive patients with BE/EAC and 7 controls revealed variable decreases in MSR1 protein levels in 3 cases (FIGS. 6A-B). All 5 MSR1-mutation positive patients had increased CCND1 levels compared with controls (FIGS. 6A-B). Barrett esophagus tissues from patients who were mutation-positive showed increased nuclear expression of CCND1 by immunohistochemistry compared with control esophageal specimens (FIGS. 7A-B). We then proceeded with the converse experiment by over-expressing wild-type MSR1 in HEK293 cells, resulting in decreased CCND1 protein (FIGS. 6A-B).

### Additional Description of the Methods

Definitions for Selection of BE/EAC Patients

The cases were recruited from adult patients with a known diagnosis of long-segment BE undergoing surveillance endoscopy, patients with a recent diagnosis of long-segment BE, and patients with a diagnosis of EAC undergoing diagnostic or therapeutic EGD in the endoscopy suites at the participating hospitals. BE was defined as a segment of salmon-colored mucosa in the esophagus at endoscopy with biopsies' demonstrating specialized columnar epithelium or intestinal metaplasia. EAC was defined as a tumor mass arising in the esophagus and confirmed on histopathologic examination.

Familial BE (FBE) was defined as having a first- or second-degree relative with long segment BE, EAC, or gastroesophageal junction adenocarcinoma (GEJAC) whose diagnosis was confirmed by review of endoscopy and histology reports. Early onset of BE/EAC was defined as BE occurring under 40 or EAC under 50.

Identification of Loci Linked to BE/EAC Using Genome-Wide Mapping Methods Combining Linkage and Association Analysis

Genotyping data were prepared for linkage analysis by reducing the number of SNPs included to only those present at 1-centiMorgan (cM) intervals within the 50K XbaI array, resulting in a total of 1,866 SNPs. Prior to linkage scanning, all pedigree errors, marker inconsistencies, and genotyping problems were corrected.

Two model-free linkage analysis methods were performed using the LODPAL and SIBPAL programs in S.A.G.E. LODPAL requires a binary trait and uses a general conditional logistic model that allows for affected-relative pairs, thereby allowing the inclusion of discordant sibpairs, to test for parent-of-origin identity-by-descent (IBD) allele sharing. SIBPAL uses the Haseman-Elston regression to perform linear regression-based modeling of sibpair traits to test whether the proportion of alleles-shared IBD by concordant siblings is greater than that shared by discordant sibling pairs. IBD marker allele sharing was estimated by GENIBD in S.A.G.E. and is used in both linkage analysis methods. The binary trait considered in both methods was affectation status, giving the phenotypes “affected” or “unaffected” with BE and/or EAC as different quantitative scores. Each chromosome was analyzed separately.

Chromosomal regions of linkage found significant in both linkage methods were considered “potentially interesting”. Peaks that were only significant in LODPAL were viewed as potential false positives due to their assumption of sibpair independence, and were thus not considered as candidate regions.

Independent Validation and Fine Mapping Significant Regions

Given the results of the exploratory linkage-association analyses above, validation association analyses, and subsequently fine mapping, were performed based on both the strength of the genetic evidence from linkage methods as well as independent association analyses (LOD scores and significant P values). Power calculations based on the above pilot suggested that >175 cases and controls, irrespective of familial status, should yield sufficient power (p>0.8) to validate or refute candidate linkage associations. Accordingly, SNP mapping was performed on an independent series totaling 176 BE/EAC patients of white European origin and 200 ancestry-matched population controls using the denser Illumina Human610-Quad BeadChips.

Genotyping and QC

Germline genomic DNA samples obtained from white blood cells were genotyped using Human610-Quad BeadChips, after which the resulting genotypes were subjected to routine QC: determination of missing genotype rate, testing for non-random genotyping failure, Hardy-Weinberg equilibrium, genotype call rates, MAF of 3-5%, and finally checking for contamination due to pipetting errors. Samples were screened and selected only if they had a minimum 95% successful genotype call rate. SNPs with minor allele frequencies (MAF) less than 3%, departures from Hardy-Weinberg equilibrium (HWE test, p<0.01), and missingness per SNP greater than 5% were excluded from further analyses.

Assessment of Population Stratification

Population stratification reflects differences in allele frequencies between cases and controls due to systematic differences in ancestry rather than association of genes with disease. As a result, false-positive associations with markers that are in linkage disequilibrium with a causal gene, that arises in recently admixed populations, can arise and often not replicable. Failure to account for population substructure may lead to both false positive and false negative SNP-disease associations. Therefore, we first ensured that the cases and the controls were matched based on European ancestry, where BE/EAC is the most prevalent compared to other ancestries. In addition, to determine if subjects had any traces of admixed origin, we used the principal components analysis (PCA) module contained in EigenStrat.

SNP-Association Analysis for Validation and Fine Mapping

We performed an association analysis to identify a set of candidate SNPs that were associated with BE/EAC. We applied logistic regression models where each SNP was used as a predictor. We included gender as a covariate and BE/EAC phenotype as our response variable. Different genetic models were considered for each locus, including co-dominant, dominant, recessive, overdominant, and additive. For each particular phenotype, the best genetic model for each SNP was selected based on the Akaike information criterion (AIC) for the fitted models. In addition to all these models, allelic association would be considered if more significant. To account for multiple comparisons, we used the FDR approach.

Moving Window Haplotype Analysis

Haplotype analysis was done using the replication dataset [N=376 (176 cases and 200 controls)] using PLINK. We defined sliding window of fixed haplotype size (i.e., 3-5 SNPs), which automatically slides through all SNPs on the chromosome, to predict the most likely haplotypes. The null hypothesis was that there was no association of the haplotypes with BE/EAC, under the assumption that the haplotypes were sampled independently. For all the windows, genotypic information was used to generate haplotypic frequencies from all possible pre-defined haplotype widths, which potential harbored a susceptibility marker or locus.

We used this approach to identify haplotypes that were significantly associated with the BE/EAC. Smaller sliding windows (3 and 4 SNPs) were first utilized, after which we proceeded to use larger windows (5 SNPs) only when there were positive signals from the smaller windows. This systematic process reduced the number of tests considerably for each analysis type. The validity of the haplotypes predicted in our analysis have been previously confirmed by several methods. For an accurate type I error, an empirical P value that corrects for testing multiple marker locations was obtained by permuting (10,000 permutations) the affection status across the individual genotypes as described in PLINK.

Integrating Genetic Information from Significant SNP/Haplotype Regions with Publicly Available BE/EAC Expression Array Datasets

To focus on biologically plausible genes, i.e., one or a subset of all genes, located within or in proximity to significant SNPs/haplotypes, we integrated our significant SNPs/haplotype regions with gene expression data were derived from a publicly available microarray analysis of 19 total samples: 7 Barrett's esophagus without dysplasia, 7 matched normal esophageal, and 5 small intestinal biopsy specimens (i.e., GDS3472 or GSE13083). We then performed unsupervised hierarchical clustering13 of genes within 250 kb on either side of or including significant SNPs and haplotypes across BE/EAC and unaffected individuals (FIGS. 5A-C; Tables 1 and 2).

Systematic Identification of Biologically Plausible Candidate Genes and Screening for Mutations in Selected Candidate Genes in BE/EAC Cases

After statistical prioritization of candidate genes and respective unique gene expressional signatures, as well as significant differentiation between cases and controls, plausible biological roles of genes relevant to BE/EAC were designated. The final list of candidate genes (“priority” candidate genes) were then scanned for germline sequence variant using a combination of the LightScanner (Idaho Technology Inc. Salt Lake City, Utah) and Sanger sequencing (ABI-3730x1) in BE/EAC cases and compared with ancestry-matched population controls (Tables 1 and 2). PCR reactions were performed on genomic DNA extracted from peripheral blood white cells in 96-well plates suitable for high-resolution melting (HRM) analysis (Eppendorf twin.tec PCR plates (Hamburg, Germany), covered with a mineral oil overlay. The HRM thermal cycling protocol consisted of 1 cycle at 95° C. for 2 minutes; 37 cycles of 95° C. for 30 seconds, 30 second hold at Tm as determined by optimization of primers; then heteroduplexes were generated by 1 cycle of 95° C. for 30 seconds followed by 1 cycle of 25° C. for 30 seconds. Next, analysis of melting curves was performed with standard LightScanner software (version 2.0). LightScanner variants obtained were then confirmed by Sanger sequencing (ABI 3730x1). Primers (oligonucleotides) were designed using LightScanner Primer Design Software (Idaho Technology, Salt Lake City, Utah) to flank the coding regions and encompassed the exon-intron boundaries of all candidate genes, as shown in Table 3.

Quantifying MSR1 and Cyclin D1 Protein Levels in BE/EAC Patients

Immortalized lymphoblastoid cells obtained from BE patients and normal controls were cultured in DMEM with 20% Fetal Bovine Serum. Cells were harvested by centrifugation at 4° C. After washing twice with ice-cold phosphate-buffered saline, cells were lysed with M-PER Mammalian Protein Extraction Reagent (Cat#78501; Thermo Fisher Scientific Inc. Fremont, Calif.), and 20-μg aliquots of total cellular protein were applied to SDS-PAGE gels. Antibodies specific to Cyclin D1 (Cat#2926, Cell signaling Technology, Inc., Danvers, Mass.), MSR1 (Cat# ab55508, Abcam) and α-tubulin (Cat# T6074, Sigma Aldrich. St. Louis, Mo.) were used for Western blotting.

Cyclin D1 Immunohistochemical Analysis

Cyclin D1 immunohistochemical analysis was performed with an avidin-biotin complex immunoperoxidase technique. After antigen retrieval and serum blocking, the primary antibody, mouse monoclonal anti-human cyclin D1 (Cat.#RM-9104-S; Thermo Fisher Scientific Inc. Fremont, Calif.) was applied using a 1:50 dilution and incubated overnight at 4° C. The secondary (biotinylated) antibodies were detected using the Vectastain Elite ABC kit (Vector laboratories, Burlingame, Calif.) according to the manufacturer's instructions. Color development was accomplished using peroxidase-conjugate and DAB. Slides were then counterstained with hematoxylin.

From the above description of the application, those skilled in the art will perceive improvements, changes and modifications. Such improvements, changes, and modifications are within the skill of those in the art and are intended to be covered by the appended claims. All patents, patent applications, and publications cited herein are incorporated by reference in their entirety.

Having described the invention, the following is claimed:

1. An isolated nucleic acid molecule comprising a polynucleotide sequence selected from the group consisting of SEQ ID NO: 7, SEQ ID NO: 8, SEQ ID NO: 13 and SEQ ID NO: 17.

2. An isolated polypeptide molecule comprising an amino acid sequence selected from the group consisting of SEQ ID NO: 9, SEQ ID NO: 10, SEQ ID NO: 14 and SEQ ID NO: 18.

3. An isolated antibody that specifically binds to a polypeptide molecule having an amino acid sequence selected from the group consisting of SEQ ID NO: 9, SEQ ID NO: 10, SEQ ID NO: 14 and SEQ ID NO: 18.

4. A method for predicting a subject's risk of developing an esophageal cancer, a precursor lesion, or both, the method comprising the steps of:
obtaining a biological sample from the subject;
determining in the biological sample the presence of at least one germline mutation; and
determining that the subject is at increased risk of an esophageal cancer, a precursor lesion, or both, due the presence of the at least one germline mutation.

5. The method of claim 4, wherein the precursor lesion is Barrett's esophagus (BE) and the esophageal cancer is esophageal adenocarcinoma (EAC).

6. The method of claim 4, wherein the at least one germline mutation is present in one or more genes selected from the group consisting of macrophage scavenger receptor 1 (MSR1), activating signal cointegrator 1 complex subunit 1 (ASCC1), and collagen triple-helix repeat-containing 1 (CTHRC1).

7. The method of claim 6, wherein the at least one germline mutation is a missense mutation or a nonsense mutation.

8. The method of claim 6, wherein the at least one germline mutation comprises a missense mutation in exon 5 of MSR1.

9. The method of claim 8, wherein the missense mutation includes a 760C>G mutation, which leads to a Leu254Val amino acid change.

10. The method of claim 6, wherein the at least one germline mutation comprises a nonsense mutation in exon 6 of MSR1.

11. The method of claim 10, wherein the nonsense mutation comprises a 877C>T mutation, which leads to an Arg293X amino acid change.

12. The method of claim 6, wherein the at least one germline mutation comprises a missense mutation in exon 1 of CTHRC1.

13. The method of claim 12, wherein the missense mutation includes a 131A>C mutation, which leads to a Gln44Pro amino acid change.

14. The method of claim 6, wherein the at least one germline mutation comprises a missense mutation in exon 8 of ASCC1.

15. The method of claim 14, wherein the missense mutation includes 869A>G, which leads to an Asn290Ser amino acid change.

16. A method for determining a treatment strategy for a subject, the method comprising the step of:
predicting the subject's risk for developing an esophageal cancer, a precursor lesion, or both, by the method of claim 4; and
if the subject exhibits a low risk of developing an esophageal cancer, a precursor lesion, or both, deciding to perform a first therapeutic intervention; or
if the subject exhibits a high risk of developing an esophageal cancer, a precursor lesion, or both, deciding to perform a second therapeutic intervention.

17. A method for treating a subject with an esophageal cancer, a precursor lesion, or both, the method comprising the steps of:
obtaining a biological sample from the subject;
determining in the biological sample the presence of at least one germline mutation; and
administering a therapeutic intervention to the subject when the presence of one or more germline mutations is detected in the biological sample.

18. A kit for performing the method of claim 4.

19. The kit of claim 18, comprising instructions for performing the method.

20. The kit of claim 19, comprising a probe for detecting the presence of at least one germline mutation.

21. The kit of claim 20, wherein the probe is a primer pair selected from the group consisting of SEQ ID NO: 19, SEQ ID NO: 20, SEQ ID NO: 21, SEQ ID NO: 22, SEQ ID NO: 23, SEQ ID NO: 24, SEQ ID NO: 25 and SEQ ID NO: 26.

22. The kit of claim 20, wherein the probe is an antibody reactive against Cyclin D1.

23. The kit of claim 20, wherein the probe is an antibody that specifically binds to a polypeptide molecule having the amino acid sequence of SEQ ID NO: 9, SEQ ID NO: 10, SEQ ID NO: 14 or SEQ ID NO: 18.


SEQUENCE LISTING (38 sequences)

SEQ ID NO: 1 - DNA, 1356 bp, Homo sapiens
atggagcagtgggatcactttcacaatcaacaggaggacactgatagctgctccgaatctgtgaaatttgatgctcgctcaatgacagctttgcttcctccgaatcctaaaaacagcccttcccttcaagagaaactgaagtccttcaaagctgcactgattgccctttacctcctcgtgtttgcagttctcatccctctcattggaatagtggcagctcaactcctgaagtgggaaacgaagaattgctcagttagttcaactaatgcaaatgatataactcaaagtctcacgggaaaaggaaatgacagcgaagaggaaatgagatttcaagaagtctttatggaacacatgagcaacatggagaagagaatccagcatattttagacatggaagccaacctcatggacacagagcatttccaaaatttcagcatgacaactgatcaaagatttaatgacattcttctgcagctaagtaccttgttttcctcagtccagggacatgggaatgcaatagatgaaatctccaagtccttaataagtttgaataccacattgcttgatttgcagctcaacatagaaaatctgaatggcaaaatccaagagaataccttcaaacaacaagaggaaatcagtaaattagaggagcgtgtttacaatgtatcagcagaaattatggctatgaaagaagaacaagtgcatttggaacaggaaataaaaggagaagtgaaagtactgaataacatcactaatgatctcagactgaaagattgggaacattctcagaccttgagaaatatcactttaattcaaggtcctcctggacccccgggtgaaaaaggagatcgaggtcccactggagaaagtggtccacgaggatttccaggtccaataggtcctccgggtcttaaaggtgatcggggagcaattggctttcctggaagtcgaggactcccaggatatgccggaaggccaggaaattctggaccaaaaggccagaaaggggaaaaggggagtggaaacacattaactccatttacgaaagttcgactggtcggtgggagcggccctcacgaggggagggtggagatactccacagcggccagtggggtacaatttgtgacgatcgctgggaagtgcgcgttggacaggtcgtctgtaggagcttgggatacccaggtgttcaagccgtgcacaaggcagctcactttggacaaggtactggtccaatatggctgaatgaagtgttttgttttgggagagaatcatctattgaagaatgtaaaattcggcaatgggggacaagagcctgttcacattctgaagatgctggagtcacttgcactttataa

SEQ ID NO: 2 - DNA, 1077 bp, Homo sapiens
atggagcagtgggatcactttcacaatcaacaggaggacactgatagctgctccgaatctgtgaaatttgatgctcgctcaatgacagctttgcttcctccgaatcctaaaaacagcccttcccttcaagagaaactgaagtccttcaaagctgcactgattgccctttacctcctcgtgtttgcagttctcatccctctcattggaatagtggcagctcaactcctgaagtgggaaacgaagaattgctcagttagttcaactaatgcaaatgatataactcaaagtctcacgggaaaaggaaatgacagcgaagaggaaatgagatttcaagaagtctttatggaacacatgagcaacatggagaagagaatccagcatattttagacatggaagccaacctcatggacacagagcatttccaaaatttcagcatgacaactgatcaaagatttaatgacattcttctgcagctaagtaccttgttttcctcagtccagggacatgggaatgcaatagatgaaatctccaagtccttaataagtttgaataccacattgcttgatttgcagctcaacatagaaaatctgaatggcaaaatccaagagaataccttcaaacaacaagaggaaatcagtaaattagaggagcgtgtttacaatgtatcagcagaaattatggctatgaaagaagaacaagtgcatttggaacaggaaataaaaggagaagtgaaagtactgaataacatcactaatgatctcagactgaaagattgggaacattctcagaccttgagaaatatcactttaattcaaggtcctcctggacccccgggtgaaaaaggagatcgaggtcccactggagaaagtggtccacgaggatttccaggtccaataggtcctccgggtcttaaaggtgatcggggagcaattggctttcctggaagtcgaggactcccaggatatgccggaaggccaggaaattctggaccaaaaggccagaaaggggaaaaggggagtggaaacacattaagaccagtacaactcactgatcatattagggcagggccctcttaa

SEQ ID NO: 3 - DNA, 1167 bp, Homo sapiens
atggagcagtgggatcactttcacaatcaacaggaggacactgatagctgctccgaatctgtgaaatttgatgctcgctcaatgacagctttgcttcctccgaatcctaaaaacagcccttcccttcaagagaaactgaagtccttcaaagctgcactgattgccctttacctcctcgtgtttgcagttctcatccctctcattggaatagtggcagctcaactcctgaagtgggaaacgaagaattgctcagttagttcaactaatgcaaatgatataactcaaagtctcacgggaaaaggaaatgacagcgaagaggaaatgagatttcaagaagtctttatggaacacatgagcaacatggagaagagaatccagcatattttagacatggaagccaacctcatggacacagagcatttccaaaatttcagcatgacaactgatcaaagatttaatgacattcttctgcagctaagtaccttgttttcctcagtccagggacatgggaatgcaatagatgaaatctccaagtccttaataagtttgaataccacattgcttgatttgcagctcaacatagaaaatctgaatggcaaaatccaagagaataccttcaaacaacaagaggaaatcagtaaattagaggagcgtgtttacaatgtatcagcagaaattatggctatgaaagaagaacaagtgcatttggaacaggaaataaaaggagaagtgaaagtactgaataacatcactaatgatctcagactgaaagattgggaacattctcagaccttgagaaatatcactttaattcaaggtcctcctggacccccgggtgaaaaaggagatcgaggtcccactggagaaagtggtccacgaggatttccaggtccaataggtcctccgggtcttaaaggtgatcggggagcaattggctttcctggaagtcgaggactcccaggatatgccggaaggccaggaaattctggaccaaaaggccagaaaggggaaaaggggagtggaaacacattaagtactggtccaatatggctgaatgaagtgttttgttttgggagagaatcatctattgaagaatgtaaaattcggcaatgggggacaagagcctgttcacattctgaagatgctggagtcacttgcactttataa

SEQ ID NO: 4 - protein, 451 aa, Homo sapiens
MEQWDHFHNQQEDTDSCSESVKFDARSMTALLPPNPKNSPSLQEKLKSFKAALIALYLLVFAVLIPLIGIVAAQLLKWETKNCSVSSTNANDITQSLTGKGNDSEEEMRFQEVFMEHMSNMEKRIQHILDMEANLMDTEHFQNFSMTTDQRFNDILLQLSTLFSSVQGHGNAIDEISKSLISLNTTLLDLQLNIENLNGKIQENTFKQQEEISKLEERVYNVSAEIMAMKEEQVHLEQEIKGEVKVLNNITNDLRLKDWEHSQTLRNITLIQGPPGPPGEKGDRGPTGESGPRGFPGPIGPPGLKGDRGAIGFPGSRGLPGYAGRPGNSGPKGQKGEKGSGNTLTPFTKVRLVGGSGPHEGRVEILHSGQWGTICDDRWEVRVGQVVCRSLGYPGVQAVHKAAHFGQGTGPIWLNEVFCFGRESSIEECKIRQWGTRACSHSEDAGVTCTL

SEQ ID NO: 5 - protein, 358 aa, Homo sapiens
MEQWDHFHNQQEDTDSCSESVKFDARSMTALLPPNPKNSPSLQEKLKSFKAALIALYLLVFAVLIPLIGIVAAQLLKWETKNCSVSSTNANDITQSLTGKGNDSEEEMRFQEVFMEHMSNMEKRIQHILDMEANLMDTEHFQNFSMTTDQRFNDILLQLSTLFSSVQGHGNAIDEISKSLISLNTTLLDLQLNIENLNGKIQENTFKQQEEISKLEERVYNVSAEIMAMKEEQVHLEQEIKGEVKVLNNITNDLRLKDWEHSQTLRNITLIQGPPGPPGEKGDRGPTGESGPRGFPGPIGPPGLKGDRGAIGFPGSRGLPGYAGRPGNSGPKGQKGEKGSGNTLRPVQLTDHIRAGPS

SEQ ID NO: 6 - protein, 388 aa, Homo sapiens
MEQWDHFHNQQEDTDSCSESVKFDARSMTALLPPNPKNSPSLQEKLKSFKAALIALYLLVFAVLIPLIGIVAAQLLKWETKNCSVSSTNANDITQSLTGKGNDSEEEMRFQEVFMEHMSNMEKRIQHILDMEANLMDTEHFQNFSMTTDQRFNDILLQLSTLFSSVQGHGNAIDEISKSLISLNTTLLDLQLNIENLNGKIQENTFKQQEEISKLEERVYNVSAEIMAMKEEQVHLEQEIKGEVKVLNNITNDLRLKDWEHSQTLRNITLIQGPPGPPGEKGDRGPTGESGPRGFPGPIGPPGLKGDRGAIGFPGSRGLPGYAGRPGNSGPKGQKGEKGSGNTLSTGPIWLNEVFCFGRESSIEECKIRQWGTRACSHSEDAGVTCTL

SEQ ID NO: 7 - DNA, 1356 bp, Homo sapiens
atggagcagtgggatcactttcacaatcaacaggaggacactgatagctgctccgaatctgtgaaatttgatgctcgctcaatgacagctttgcttcctccgaatcctaaaaacagcccttcccttcaagagaaactgaagtccttcaaagctgcactgattgccctttacctcctcgtgtttgcagttctcatccctctcattggaatagtggcagctcaactcctgaagtgggaaacgaagaattgctcagttagttcaactaatgcaaatgatataactcaaagtctcacgggaaaaggaaatgacagcgaagaggaaatgagatttcaagaagtctttatggaacacatgagcaacatggagaagagaatccagcatattttagacatggaagccaacctcatggacacagagcatttccaaaatttcagcatgacaactgatcaaagatttaatgacattcttctgcagctaagtaccttgttttcctcagtccagggacatgggaatgcaatagatgaaatctccaagtccttaataagtttgaataccacattgcttgatttgcagctcaacatagaaaatctgaatggcaaaatccaagagaataccttcaaacaacaagaggaaatcagtaaattagaggagcgtgtttacaatgtatcagcagaaattatggctatgaaagaagaacaagtgcatttggaacaggaaataaaaggagaagtgaaagtactgaataacatcactaatgatctcagactgaaagattgggaacattctcagaccttgagaaatatcactttaattcaaggtcctcctggacccccgggtgaaaaaggagatcgaggtcccactggagaaagtggtccatgaggatttccaggtccaataggtcctccgggtcttaaaggtgatcggggagcaattggctttcctggaagtcgaggactcccaggatatgccggaaggccaggaaattctggaccaaaaggccagaaaggggaaaaggggagtggaaacacattaactccatttacgaaagttcgactggtcggtgggagcggccctcacgaggggagggtggagatactccacagcggccagtggggtacaatttgtgacgatcgctgggaagtgcgcgttggacaggtcgtctgtaggagcttgggatacccaggtgttcaagccgtgcacaaggcagctcactttggacaaggtactggtccaatatggctgaatgaagtgttttgttttgggagagaatcatctattgaagaatgtaaaattcggcaatgggggacaagagcctgttcacattctgaagatgctggagtcacttgcactttataa

SEQ ID NO: 8 - DNA, 1356 bp, Homo sapiens
atggagcagtgggatcactttcacaatcaacaggaggacactgatagctgctccgaatctgtgaaatttgatgctcgctcaatgacagctttgcttcctccgaatcctaaaaacagcccttcccttcaagagaaactgaagtccttcaaagctgcactgattgccctttacctcctcgtgtttgcagttctcatccctctcattggaatagtggcagctcaactcctgaagtgggaaacgaagaattgctcagttagttcaactaatgcaaatgatataactcaaagtctcacgggaaaaggaaatgacagcgaagaggaaatgagatttcaagaagtctttatggaacacatgagcaacatggagaagagaatccagcatattttagacatggaagccaacctcatggacacagagcatttccaaaatttcagcatgacaactgatcaaagatttaatgacattcttctgcagctaagtaccttgttttcctcagtccagggacatgggaatgcaatagatgaaatctccaagtccttaataagtttgaataccacattgcttgatttgcagctcaacatagaaaatctgaatggcaaaatccaagagaataccttcaaacaacaagaggaaatcagtaaattagaggagcgtgtttacaatgtatcagcagaaattatggctatgaaagaagaacaagtgcatttggaacaggaaataaaaggagaagtgaaagtactgaataacatcactaatgatgtcagactgaaagattgggaacattctcagaccttgagaaatatcactttaattcaaggtcctcctggacccccgggtgaaaaaggagatcgaggtcccactggagaaagtggtccacgaggatttccaggtccaataggtcctccgggtcttaaaggtgatcggggagcaattggctttcctggaagtcgaggactcccaggatatgccggaaggccaggaaattctggaccaaaaggccagaaaggggaaaaggggagtggaaacacattaactccatttacgaaagttcgactggtcggtgggagcggccctcacgaggggagggtggagatactccacagcggccagtggggtacaatttgtgacgatcgctgggaagtgcgcgttggacaggtcgtctgtaggagcttgggatacccaggtgttcaagccgtgcacaaggcagctcactttggacaaggtactggtccaatatggctgaatgaagtgttttgttttgggagagaatcatctattgaagaatgtaaaattcggcaatgggggacaagagcctgttcacattctgaagatgctggagtcacttgcactttataa

SEQ ID NO: 9 - protein, 451 aa, Homo sapiens
  MISC_FEATURE (293)..(293): “Xaa = missing (due to STOP codon)”
MEQWDHFHNQQEDTDSCSESVKFDARSMTALLPPNPKNSPSLQEKLKSFKAALIALYLLVFAVLIPLIGIVAAQLLKWETKNCSVSSTNANDITQSLTGKGNDSEEEMRFQEVFMEHMSNMEKRIQHILDMEANLMDTEHFQNFSMTTDQRFNDILLQLSTLFSSVQGHGNAIDEISKSLISLNTTLLDLQLNIENLNGKIQENTFKQQEEISKLEERVYNVSAEIMAMKEEQVHLEQEIKGEVKVLNNITNDLRLKDWEHSQTLRNITLIQGPPGPPGEKGDRGPTGESGPXGFPGPIGPPGLKGDRGAIGFPGSRGLPGYAGRPGNSGPKGQKGEKGSGNTLTPFTKVRLVGGSGPHEGRVEILHSGQWGTICDDRWEVRVGQVVCRSLGYPGVQAVHKAAHFGQGTGPIWLNEVFCFGRESSIEECKIRQWGTRACSHSEDAGVTCTL

SEQ ID NO: 10 - protein, 451 aa, Homo sapiens
MEQWDHFHNQQEDTDSCSESVKFDARSMTALLPPNPKNSPSLQEKLKSFKAALIALYLLVFAVLIPLIGIVAAQLLKWETKNCSVSSTNANDITQSLTGKGNDSEEEMRFQEVFMEHMSNMEKRIQHILDMEANLMDTEHFQNFSMTTDQRFNDILLQLSTLFSSVQGHGNAIDEISKSLISLNTTLLDLQLNIENLNGKIQENTFKQQEEISKLEERVYNVSAEIMAMKEEQVHLEQEIKGEVKVLNNITNDVRLKDWEHSQTLRNITLIQGPPGPPGEKGDRGPTGESGPRGFPGPIGPPGLKGDRGAIGFPGSRGLPGYAGRPGNSGPKGQKGEKGSGNTLTPFTKVRLVGGSGPHEGRVEILHSGQWGTICDDRWEVRVGQVVCRSLGYPGVQAVHKAAHFGQGTGPIWLNEVFCFGRESSIEECKIRQWGTRACSHSEDAGVTCTL

SEQ ID NO: 11 - DNA, 1203 bp, Homo sapiens
atggaagttctgcgtccacagcttataagaattgatggccggaattacaggaagaatccagtccaagaacagacctatcaacatgaagaagatgaagaggacttctatcaaggctccatggagtgtgctgatgagccctgtgatgcctacgaggtggagcagaccccacaaggattccggtctactttgagggcccccagcttgctctataatctcattcacttgaacacatcaaacgactgtgggttccagaagataactttggattgtcagaatatttatacttggaagtccaggcatatagttggaaagagaggggacactaggaagaaaatagaaatggagaccaaaacttctattagcattcctaaacctggacaagacggggaaattgtaatcactggccagcatcgaaatggtgtaatttcagcccgaacacggattgatgttcttttggacacttttcgaagaaagcagcccttcactcacttccttgcctttttcctcaatgaagttgaggttcaggaaggattcctgagattccaggaggaagtactggcgaagtgctccatggatcatggggttgacagcagcattttccagaatcctaaaaagcttcatctaactattgggatgttggtgcttttgagtgaggaagagatccagcagacatgtgagatgctacagcagtgtaaagaggaattcattaatgatatttctgggggtaaacccctagaagtggagatggcagggatagaatacatgaatgatgatcctggcatggtggatgttctttacgccaaagtccatatgaaagatggctccaacaggctacaagaattagttgatcgagtgctggaacgttttcaggcatctggactaatagtgaaagagtggaatagtgtgaaactgcatgctacagttatgaatacactattcaggaaagaccccaatgctgaaggcaggtacaatctctacacagcggaaggcaaatatatcttcaaggaaagagaatcatttgatggccgaaatattttaaagagctttgccttgttgcccaggctggagtacaatgatgcaatctccgctcactgcaacctgtgcctcccgggttcaagtgattctcctgcctcagcctcccaagtagctgggattacaggtgtctctgatgcatattctcagagcctaccaggaaaatcctag

SEQ ID NO: 12 - protein, 400 aa, Homo sapiens
MEVLRPQLIRIDGRNYRKNPVQEQTYQHEEDEEDFYQGSMECADEPCDAYEVEQTPQGFRSTLRAPSLLYNLIHLNTSNDCGFQKITLDCQNIYTWKSRHIVGKRGDTRKKIEMETKTSISIPKPGQDGEIVITGQHRNGVISARTRIDVLLDTFRRKQPFTHFLAFFLNEVEVQEGFLRFQEEVLAKCSMDHGVDSSIFQNPKKLHLTIGMLVLLSEEEIQQTCEMLQQCKEEFINDISGGKPLEVEMAGIEYMNDDPGMVDVLYAKVHMKDGSNRLQELVDRVLERFQASGLIVKEWNSVKLHATVMNTLFRKDPNAEGRYNLYTAEGKYIFKERESFDGRNILKSFALLPRLEYNDAISAHCNLCLPGSSDSPASASQVAGITGVSDAYSQSLPGKS

SEQ ID NO: 13 - DNA, 1203 bp, Homo sapiens
atggaagttctgcgtccacagcttataagaattgatggccggaattacaggaagaatccagtccaagaacagacctatcaacatgaagaagatgaagaggacttctatcaaggctccatggagtgtgctgatgagccctgtgatgcctacgaggtggagcagaccccacaaggattccggtctactttgagggcccccagcttgctctataatctcattcacttgaacacatcaaacgactgtgggttccagaagataactttggattgtcagaatatttatacttggaagtccaggcatatagttggaaagagaggggacactaggaagaaaatagaaatggagaccaaaacttctattagcattcctaaacctggacaagacggggaaattgtaatcactggccagcatcgaaatggtgtaatttcagcccgaacacggattgatgttcttttggacacttttcgaagaaagcagcccttcactcacttccttgcctttttcctcaatgaagttgaggttcaggaaggattcctgagattccaggaggaagtactggcgaagtgctccatggatcatggggttgacagcagcattttccagaatcctaaaaagcttcatctaactattgggatgttggtgcttttgagtgaggaagagatccagcagacatgtgagatgctacagcagtgtaaagaggaattcattaatgatatttctgggggtaaacccctagaagtggagatggcagggatagaatacatgaatgatgatcctggcatggtggatgttctttacgccaaagtccatatgaaagatggctccaacaggctacaagaattagttgatcgagtgctggaacgttttcgggcatctggactaatagtgaaagagtggaatagtgtgaaactgcatgctacagttatgaatacactattcaggaaagaccccagtgctgaaggcaggtacaatctctacacagcggaaggcaaatatatcttcaaggaaagagaatcatttgatggccgaaatattttaaagagctttgccttgttgcccaggctggagtacaatgatgcaatctccgctcactgcaacctgtgcctcccgggttcaagtgattctcctgcctcagcctcccaagtagctgggattacaggtgtctctgatgcatattctcagagcctaccaggaaaatcctag

SEQ ID NO: 14 - protein, 400 aa, Homo sapiens
MEVLRPQLIRIDGRNYRKNPVQEQTYQHEEDEEDFYQGSMECADEPCDAYEVEQTPQGFRSTLRAPSLLYNLIHLNTSNDCGFQKITLDCQNIYTWKSRHIVGKRGDTRKKIEMETKTSISIPKPGQDGEIVITGQHRNGVISARTRIDVLLDTFRRKQPFTHFLAFFLNEVEVQEGFLRFQEEVLAKCSMDHGVDSSIFQNPKKLHLTIGMLVLLSEEEIQQTCEMLQQCKEEFINDISGGKPLEVEMAGIEYMNDDPGMVDVLYAKVHMKDGSNRLQELVDRVLERFSASGLIVKEWNSVKLHATVMNTLFRKDPSAEGRYNLYTAEGKYIFKERESFDGRNILKSFALLPRLEYNDAISAHCNLCLPGSSDSPASASQVAGITGVSDAYSQSLPGKS

SEQ ID NO: 15 - DNA, 732 bp, Homo sapiens
atgcgaccccagggccccgccgcctccccgcagcggctccgcggcctcctgctgctcctgctgctgcagctgcccgcgccgtcgagcgcctctgagatccccaaggggaagcaaaaggcgcagctccggcagagggaggtggtggacctgtataatggaatgtgcttacaagggccagcaggagtgcctggtcgagacgggagccctggggccaatggcattccgggtacacctgggatcccaggtcgggatggattcaaaggagaaaagggggaatgtctgagggaaagctttgaggagtcctggacacccaactacaagcagtgttcatggagttcattgaattatggcatagatcttgggaaaattgcggagtgtacatttacaaagatgcgttcaaatagtgctctaagagttttgttcagtggctcacttcggctaaaatgcagaaatgcatgctgtcagcgttggtatttcacattcaatggagctgaatgttcaggacctcttcccattgaagctataatttatttggaccaaggaagccctgaaatgaattcaacaattaatattcatcgcacttcttctgtggaaggactttgtgaaggaattggtgctggattagtggatgttgctatctgggttggtacttgttcagattacccaaaaggagatgcttctactggatggaattcagtttctcgcatcattattgaagaactaccaaaataa

SEQ ID NO: 16 - protein, 243 aa, Homo sapiens
MRPQGPAASPQRLRGLLLLLLLQLPAPSSASEIPKGKQKAQLRQREVVDLYNGMCLQGPAGVPGRDGSPGANGIPGTPGIPGRDGFKGEKGECLRESFEESWTPNYKQCSWSSLNYGIDLGKIAECTFTKMRSNSALRVLFSGSLRLKCRNACCQRWYFTFNGAECSGPLPIEAIIYLDQGSPEMNSTINIHRTSSVEGLCEGIGAGLVDVAIWVGTCSDYPKGDASTGWNSVSRIIIEELPK

SEQ ID NO: 17 - DNA, 732 bp, Homo sapiens
atgcgaccccagggccccgccgcctccccgcagcggctccgcggcctcctgctgctcctgctgctgcagctgcccgcgccgtcgagcgcctctgagatccccaaggggaagcaaaaggcgcagctccggccgagggaggtggtggacctgtataatggaatgtgcttacaagggccagcaggagtgcctggtcgagacgggagccctggggccaatggcattccgggtacacctgggatcccaggtcgggatggattcaaaggagaaaagggggaatgtctgagggaaagctttgaggagtcctggacacccaactacaagcagtgttcatggagttcattgaattatggcatagatcttgggaaaattgcggagtgtacatttacaaagatgcgttcaaatagtgctctaagagttttgttcagtggctcacttcggctaaaatgcagaaatgcatgctgtcagcgttggtatttcacattcaatggagctgaatgttcaggacctcttcccattgaagctataatttatttggaccaaggaagccctgaaatgaattcaacaattaatattcatcgcacttcttctgtggaaggactttgtgaaggaattggtgctggattagtggatgttgctatctgggttggtacttgttcagattacccaaaaggagatgcttctactggatggaattcagtttctcgcatcattattgaagaactaccaaaataa

SEQ ID NO: 18 - protein, 243 aa, Homo sapiens
MRPQGPAASPQRLRGLLLLLLLQLPAPSSASEIPKGKQKAQLRPREVVDLYNGMCLQGPAGVPGRDGSPGANGIPGTPGIPGRDGFKGEKGECLRESFEESWTPNYKQCSWSSLNYGIDLGKIAECTFTKMRSNSALRVLFSGSLRLKCRNACCQRWYFTFNGAECSGPLPIEAIIYLDQGSPEMNSTINIHRTSSVEGLCEGIGAGLVDVAIWVGTCSDYPKGDASTGWNSVSRIIIEELPK

SEQ ID NO: 19 - DNA, 28 bp, Artificial Sequence
  FORWARD PRIMER EXON 5 MSRI
gataattatgtatgtaacaaaggctgtg

SEQ ID NO: 20 - DNA, 28 bp, Artificial Sequence
  REVERSE PRIMER EXON 5 MSRI
cataggatttcaaatatcaaatctctgc

SEQ ID NO: 21 - DNA, 22 bp, Artificial Sequence
  FORWARD PRIMER EXON 5 MSRI
cttgacagatgactaacccatt

SEQ ID NO: 22 - DNA, 20 bp, Artificial Sequence
  REVERSE PRIMER EXON 6 MSRI
ccctacacatgtacctggat

SEQ ID NO: 23 - DNA, 23 bp, Artificial Sequence
  FORWARD PRIMER EXON 8 MSRI
cgcatgttcagaagtttactaag

SEQ ID NO: 24 - DNA, 18 bp, Artificial Sequence
  REVERSE PRIMER EXON 8 ASCCI
ctcagagatgccacaagg

SEQ ID NO: 25 - DNA, 18 bp, Artificial Sequence
  FORWARD PRIMER EXON 1 CTHRC 1
cgttcctctcctcggtct

SEQ ID NO: 26 - DNA, 16 bp, Artificial Sequence
  REVERSE PRIMER EXON 1 CTHRC
gccaggtcccctccac

SEQ ID NO: 27 - DNA, 1077 bp, Homo sapiens
atggagcagtgggatcactttcacaatcaacaggaggacactgatagctgctccgaatctgtgaaatttgatgctcgctcaatgacagctttgcttcctccgaatcctaaaaacagcccttcccttcaagagaaactgaagtccttcaaagctgcactgattgccctttacctcctcgtgtttgcagttctcatccctctcattggaatagtggcagctcaactcctgaagtgggaaacgaagaattgctcagttagttcaactaatgcaaatgatataactcaaagtctcacgggaaaaggaaatgacagcgaagaggaaatgagatttcaagaagtctttatggaacacatgagcaacatggagaagagaatccagcatattttagacatggaagccaacctcatggacacagagcatttccaaaatttcagcatgacaactgatcaaagatttaatgacattcttctgcagctaagtaccttgttttcctcagtccagggacatgggaatgcaatagatgaaatctccaagtccttaataagtttgaataccacattgcttgatttgcagctcaacatagaaaatctgaatggcaaaatccaagagaataccttcaaacaacaagaggaaatcagtaaattagaggagcgtgtttacaatgtatcagcagaaattatggctatgaaagaagaacaagtgcatttggaacaggaaataaaaggagaagtgaaagtactgaataacatcactaatgatctcagactgaaagattgggaacattctcagaccttgagaaatatcactttaattcaaggtcctcctggacccccgggtgaaaaaggagatcgaggtcccactggagaaagtggtccatgaggatttccaggtccaataggtcctccgggtcttaaaggtgatcggggagcaattggctttcctggaagtcgaggactcccaggatatgccggaaggccaggaaattctggaccaaaaggccagaaaggggaaaaggggagtggaaacacattaagaccagtacaactcactgatcatattagggcagggccctcttaa

SEQ ID NO: 28 - DNA, 1077 bp, Homo sapiens
atggagcagtgggatcactttcacaatcaacaggaggacactgatagctgctccgaatctgtgaaatttgatgctcgctcaatgacagctttgcttcctccgaatcctaaaaacagcccttcccttcaagagaaactgaagtccttcaaagctgcactgattgccctttacctcctcgtgtttgcagttctcatccctctcattggaatagtggcagctcaactcctgaagtgggaaacgaagaattgctcagttagttcaactaatgcaaatgatataactcaaagtctcacgggaaaaggaaatgacagcgaagaggaaatgagatttcaagaagtctttatggaacacatgagcaacatggagaagagaatccagcatattttagacatggaagccaacctcatggacacagagcatttccaaaatttcagcatgacaactgatcaaagatttaatgacattcttctgcagctaagtaccttgttttcctcagtccagggacatgggaatgcaatagatgaaatctccaagtccttaataagtttgaataccacattgcttgatttgcagctcaacatagaaaatctgaatggcaaaatccaagagaataccttcaaacaacaagaggaaatcagtaaattagaggagcgtgtttacaatgtatcagcagaaattatggctatgaaagaagaacaagtgcatttggaacaggaaataaaaggagaagtgaaagtactgaataacatcactaatgatgtcagactgaaagattgggaacattctcagaccttgagaaatatcactttaattcaaggtcctcctggacccccgggtgaaaaaggagatcgaggtcccactggagaaagtggtccacgaggatttccaggtccaataggtcctccgggtcttaaaggtgatcggggagcaattggctttcctggaagtcgaggactcccaggatatgccggaaggccaggaaattctggaccaaaaggccagaaaggggaaaaggggagtggaaacacattaagaccagtacaactcactgatcatattagggcagggccctcttaa

SEQ ID NO: 29 - DNA, 1167 bp, Homo sapiens
atggagcagtgggatcactttcacaatcaacaggaggacactgatagctgctccgaatctgtgaaatttgatgctcgctcaatgacagctttgcttcctccgaatcctaaaaacagcccttcccttcaagagaaactgaagtccttcaaagctgcactgattgccctttacctcctcgtgtttgcagttctcatccctctcattggaatagtggcagctcaactcctgaagtgggaaacgaagaattgctcagttagttcaactaatgcaaatgatataactcaaagtctcacgggaaaaggaaatgacagcgaagaggaaatgagatttcaagaagtctttatggaacacatgagcaacatggagaagagaatccagcatattttagacatggaagccaacctcatggacacagagcatttccaaaatttcagcatgacaactgatcaaagatttaatgacattcttctgcagctaagtaccttgttttcctcagtccagggacatgggaatgcaatagatgaaatctccaagtccttaataagtttgaataccacattgcttgatttgcagctcaacatagaaaatctgaatggcaaaatccaagagaataccttcaaacaacaagaggaaatcagtaaattagaggagcgtgtttacaatgtatcagcagaaattatggctatgaaagaagaacaagtgcatttggaacaggaaataaaaggagaagtgaaagtactgaataacatcactaatgatctcagactgaaagattgggaacattctcagaccttgagaaatatcactttaattcaaggtcctcctggacccccgggtgaaaaaggagatcgaggtcccactggagaaagtggtccatgaggatttccaggtccaataggtcctccgggtcttaaaggtgatcggggagcaattggctttcctggaagtcgaggactcccaggatatgccggaaggccaggaaattctggaccaaaaggccagaaaggggaaaaggggagtggaaacacattaagtactggtccaatatggctgaatgaagtgttttgttttgggagagaatcatctattgaagaatgtaaaattcggcaatgggggacaagagcctgttcacattctgaagatgctggagtcacttgcactttataa

SEQ ID NO: 30 - DNA, 1167 bp, Homo sapiens
atggagcagtgggatcactttcacaatcaacaggaggacactgatagctgctccgaatctgtgaaatttgatgctcgctcaatgacagctttgcttcctccgaatcctaaaaacagcccttcccttcaagagaaactgaagtccttcaaagctgcactgattgccctttacctcctcgtgtttgcagttctcatccctctcattggaatagtggcagctcaactcctgaagtgggaaacgaagaattgctcagttagttcaactaatgcaaatgatataactcaaagtctcacgggaaaaggaaatgacagcgaagaggaaatgagatttcaagaagtctttatggaacacatgagcaacatggagaagagaatccagcatattttagacatggaagccaacctcatggacacagagcatttccaaaatttcagcatgacaactgatcaaagatttaatgacattcttctgcagctaagtaccttgttttcctcagtccagggacatgggaatgcaatagatgaaatctccaagtccttaataagtttgaataccacattgcttgatttgcagctcaacatagaaaatctgaatggcaaaatccaagagaataccttcaaacaacaagaggaaatcagtaaattagaggagcgtgtttacaatgtatcagcagaaattatggctatgaaagaagaacaagtgcatttggaacaggaaataaaaggagaagtgaaagtactgaataacatcactaatgatgtcagactgaaagattgggaacattctcagaccttgagaaatatcactttaattcaaggtcctcctggacccccgggtgaaaaaggagatcgaggtcccactggagaaagtggtccacgaggatttccaggtccaataggtcctccgggtcttaaaggtgatcggggagcaattggctttcctggaagtcgaggactcccaggatatgccggaaggccaggaaattctggaccaaaaggccagaaaggggaaaaggggagtggaaacacattaagtactggtccaatatggctgaatgaagtgttttgttttgggagagaatcatctattgaagaatgtaaaattcggcaatgggggacaagagcctgttcacattctgaagatgctggagtcacttgcactttataa

SEQ ID NO: 31 - protein, 358 aa, Homo sapiens
  MISC._FEATURE (293)..(293): “Xaa = missing (due to STOP codon)”
MEQWDHFHNQQEDTDSCSESVKFDARSMTALLPPNPKNSPSLQEKLKSFKAALIALYLLVFAVLIPLIGIVAAQLLKWETKNCSVSSTNANDITQSLTGKGNDSEEEMRFQEVFMEHMSNMEKRIQHILDMEANLMDTEHFQNFSMTTDQRFNDILLQLSTLFSSVQGHGNAIDEISKSLISLNTTLLDLQLNIENLNGKIQENTFKQQEEISKLEERVYNVSAEIMAMKEEQVHLEQEIKGEVKVLNNITNDLRLKDWEHSQTLRNITLIQGPPGPPGEKGDRGPTGESGPXGFPGPIGPPGLKGDRGAIGFPGSRGLPGYAGRPGNSGPKGQKGEKGSGNTLRPVQLTDHIRAGPS

SEQ ID NO: 32 - protein, 358 aa, Homo sapiens
MEQWDHFHNQQEDTDSCSESVKFDARSMTALLPPNPKNSPSLQEKLKSFKAALIALYLLVFAVLIPLIGIVAAQLLKWETKNCSVSSTNANDITQSLTGKGNDSEEEMRFQEVFMEHMSNMEKRIQHILDMEANLMDTEHFQNFSMTTDQRFNDILLQLSTLFSSVQGHGNAIDEISKSLISLNTTLLDLQLNIENLNGKIQENTFKQQEEISKLEERVYNVSAEIMAMKEEQVHLEQEIKGEVKVLNNITNDVRLKDWEHSQTLRNITLIQGPPGPPGEKGDRGPTGESGPRGFPGPIGPPGLKGDRGAIGFPGSRGLPGYAGRPGNSGPKGQKGEKGSGNTLRPVQLTDHIRAGPS

SEQ ID NO: 33 - protein, 388 aa, Homo sapiens
  MISC._FEATURE (293)..(293): “Xaa = missing (due to STOP codon)”
MEQWDHFHNQQEDTDSCSESVKFDARSMTALLPPNPKNSPSLQEKLKSFKAALIALYLLVFAVLIPLIGIVAAQLLKWETKNCSVSSTNANDITQSLTGKGNDSEEEMRFQEVFMEHMSNMEKRIQHILDMEANLMDTEHFQNFSMTTDQRFNDILLQLSTLFSSVQGHGNAIDEISKSLISLNTTLLDLQLNIENLNGKIQENTFKQQEEISKLEERVYNVSAEIMAMKEEQVHLEQEIKGEVKVLNNITNDLRLKDWEHSQTLRNITLIQGPPGPPGEKGDRGPTGESGPXGFPGPIGPPGLKGDRGAIGFPGSRGLPGYAGRPGNSGPKGQKGEKGSGNTLSTGPIWLNEVFCFGRESSIEECKIRQWGTRACSHSEDAGVTCTL

SEQ ID NO: 34 - protein, 388 aa, Homo sapiens
MEQWDHFHNQQEDTDSCSESVKFDARSMTALLPPNPKNSPSLQEKLKSFKAALIALYLLVFAVLIPLIGIVAAQLLKWETKNCSVSSTNANDITQSLTGKGNDSEEEMRFQEVFMEHMSNMEKRIQHILDMEANLMDTEHFQNFSMTTDQRFNDILLQLSTLFSSVQGHGNAIDEISKSLISLNTTLLDLQLNIENLNGKIQENTFKQQEEISKLEERVYNVSAEIMAMKEEQVHLEQEIKGEVKVLNNITNDVRLKDWEHSQTLRNITLIQGPPGPPGEKGDRGPTGESGPRGFPGPIGPPGLKGDRGAIGFPGSRGLPGYAGRPGNSGPKGQKGEKGSGNTLSTGPIWLNEVFCFGRESSIEECKIRQWGTRACSHSEDAGVTCTL

SEQ ID NO: 35 - DNA, 1074 bp, Homo sapiens
atggaagttctgcgtccacagcttataagaattgatggccggaattacaggaagaatccagtccaagaacagacctatcaacatgaagaagatgaagaggacttctatcaaggctccatggagtgtgctgatgagccctgtgatgcctacgaggtggagcagaccccacaaggattccggtctactttgagggcccccagcttgctctataagcatatagttggaaagagaggggacactaggaagaaaatagaaatggagaccaaaacttctattagcattcctaaacctggacaagacggggaaattgtaatcactggccagcatcgaaatggtgtaatttcagcccgaacacggattgatgttcttttggacacttttcgaagaaagcagcccttcactcacttccttgcctttttcctcaatgaagttgaggttcaggaaggattcctgagattccaggaggaagtactggcgaagtgctccatggatcatggggttgacagcagcattttccagaatcctaaaaagcttcatctaactattgggatgttggtgcttttgagtgaggaagagatccagcagacatgtgagatgctacagcagtgtaaagaggaattcattaatgatatttctgggggtaaacccctagaagtggagatggcagggatagaatacatgaatgatgatcctggcatggtggatgttctttacgccaaagtccatatgaaagatggctccaacaggctacaagaattagttgatcgagtgctggaacgttttcaggcatctggactaatagtgaaagagtggaatagtgtgaaactgcatgctacagttatgaatacactattcaggaaagaccccaatgctgaaggcaggtacaatctctacacagcggaaggcaaatatatcttcaaggaaagagaatcatttgatggccgaaatattttaaagttgtttgagaacttctactttggctccctaaagctgaattcaattcacatctctcagaggttcaccgtagacagctttggaaactacgcttcctgtggacaaattgacttctcctga

SEQ ID NO: 36 - protein, 357 aa, Homo sapiens
MEVLRPQLIRIDGRNYRKNPVQEQTYQHEEDEEDFYQGSMECADEPCDAYEVEQTPQGFRSTLRAPSLLYKHIVGKRGDTRKKIEMETKTSISIPKPGQDGEIVITGQHRNGVISARTRIDVLLDTFRRKQPFTHFLAFFLNEVEVQEGFLRFQEEVLAKCSMDHGVDSSIFQNPKKLHLTIGMLVLLSEEEIQQTCEMLQQCKEEFINDISGGKPLEVEMAGIEYMNDDPGMVDVLYAKVHMKDGSNRLQELVDRVLERFQASGLIVKEWNSVKLHATVMNTLFRKDPNAEGRYNLYTAEGKYIFKERESFDGRNILKLFENFYFGSLKLNSIHISQRFTVDSFGNYASCGQIDFS

SEQ ID NO: 37 - DNA, 1074 bp, Homo sapiens
atggaagttctgcgtccacagcttataagaattgatggccggaattacaggaagaatccagtccaagaacagacctatcaacatgaagaagatgaagaggacttctatcaaggctccatggagtgtgctgatgagccctgtgatgcctacgaggtggagcagaccccacaaggattccggtctactttgagggcccccagcttgctctataagcatatagttggaaagagaggggacactaggaagaaaatagaaatggagaccaaaacttctattagcattcctaaacctggacaagacggggaaattgtaatcactggccagcatcgaaatggtgtaatttcagcccgaacacggattgatgttcttttggacacttttcgaagaaagcagcccttcactcacttccttgcctttttcctcaatgaagttgaggttcaggaaggattcctgagattccaggaggaagtactggcgaagtgctccatggatcatggggttgacagcagcattttccagaatcctaaaaagcttcatctaactattgggatgttggtgcttttgagtgaggaagagatccagcagacatgtgagatgctacagcagtgtaaagaggaattcattaatgatatttctgggggtaaacccctagaagtggagatggcagggatagaatacatgaatgatgatcctggcatggtggatgttctttacgccaaagtccatatgaaagatggctccaacaggctacaagaattagttgatcgagtgctggaacgttttcaggcatctggactaatagtgaaagagtggaatagtgtgaaactgcatgctacagttatgaatacactattcaggaaagaccccagtgctgaaggcaggtacaatctctacacagcggaaggcaaatatatcttcaaggaaagagaatcatttgatggccgaaatattttaaagttgtttgagaacttctactttggctccctaaagctgaattcaattcacatctctcagaggttcaccgtagacagctttggaaactacgcttcctgtggacaaattgacttctcctga

SEQ ID NO: 38 - protein, 357 aa, Homo sapiens [sequence omitted]